(19) United States
(12) Patent Application Publication
(10) Pub. No.: US 2006/0106038 A1
(43) Pub. Date: May 18, 2006

(54) Methods for treating and/or preventing aberrant proliferation of hematopoietic cells

(75) Inventors: Didier Bouscary, Paris (FR); Joel S. Hayflick, Seattle, WA (US); Catherine Lacombe, Paris (FR); Patrick Mayeux, Paris (FR)

Correspondence Address: MARSHALL, GERSTEIN & BORUN LLP, 233 S. WACKER DRIVE, SUITE 6300, SEARS TOWER, CHICAGO, IL, 60606, US

(73) Assignee: ICOS CORPORATION, Bothell, WA (US)

(21) Appl. No.: 11/137,901

(22) Filed: May 25, 2005

Related U.S. Application Data
(60) Provisional application No. 60/574,481, filed on May 25, 2004.
(60) Provisional application No. 60/578,683, filed on Jun. 9, 2004.

(51) Int. Cl. A61K 31/52

(52) U.S. Cl. 514/263.21

(57) ABSTRACT

The invention relates generally to methods for treating and/or preventing aberrant proliferation of hematopoietic cells. More particularly, the invention relates to methods for treating and/or preventing aberrant proliferation of hematopoietic cells comprising selectively inhibiting phosphoinositide 3-kinase delta (PI3Kδ) activity in hematopoietic cells. The methods may be used to treat any indication involving aberrant proliferation of hematopoietic cells.

### CROSS-REFERENCE TO RELATED APPLICATIONS

The benefits under 35 U.S.C. §119(e) of U.S. provisional patent application Ser. No. 60/574,481 filed May 25, 2004, and U.S. provisional patent application Ser. No. 60/578,683 filed Jun. 9, 2004, the entire disclosures of which are incorporated herein by reference, are claimed.

### FIELD OF THE INVENTION

The invention relates generally to methods for treating and/or preventing aberrant proliferation of hematopoietic cells. More particularly, the invention relates to methods for treating and/or preventing aberrant proliferation of hematopoietic cells comprising selectively inhibiting phosphoinositide 3-kinase delta (PI3Kδ) activity in hematopoietic cells.

### BACKGROUND OF THE INVENTION

Aberrant cell proliferation is cell proliferation that deviates from the normal, proper, or expected course. Aberrant cell proliferation is the hallmark of cancer.

Cancers can generally be divided into solid tumors affecting organs and/or connective tissues (including but not limited to bone and cartilage) and hematological malignancies that arise from hematopoietic cells. Hematopoietic cells typically differentiate into either lymphoid progenitor cells or myeloid progenitor cells, both of which ultimately differentiate into various mature cell types including but not limited to leukocytes. Lymphoid progenitor cell-derived cells include but are not limited to natural killer cells, T cells, B cells, and plasma cells. Myeloid progenitor cell-derived cells include but are not limited to erythrocytes (red blood cells), megakaryocytes (platelet producing cells), monocytes, macrophages, and granulocytes such as neutrophils, eosinophils, and basophils. Because the aforementioned leukocytes are integral components of the body's immune system, aberrant proliferation of hematopoietic cells can impair an individual's ability to fight infection. Additionally, aberrant proliferation of hematopoietic cells of one type often interferes with the production or survival of other hematopoietic cell types, which can result in anemia and/or thrombocytopenia.

Various disease states, disorders, and conditions (hereafter, indications) involving aberrant proliferation of hematopoietic cells (i.e, including excessive production of lymphoid progenitor cell-derived cells and/or myeloid progenitor cell-derived cells) include but are not limited to leukemias, lymphomas, myeloproliferative disorders, myelodysplastic syndromes, and plasma cell neoplasms.

Leukemias are cancers that are characterized by an uncontrolled increase in the number of at least one type of leukocyte and/or leukocyte precursor in the blood and/or bone marrow. Leukemias are generally classified as either acute or chronic, which correlates with both the tempo of the clinical course and the degree of leukocyte differentiation. In acute leukemias, the involved cell line (usually referred to as blast cells) shows little or no differentiation. In chronic leukemias, on the other hand, the involved cell line is typically more well-differentiated but immunologically incompetent. Leukemias are also further classified according to cell lineage as either myelogenous (when myeloid progenitor cell-derived cells are involved) or lymphocytic (when lymphoid progenitor cell-derived cells are involved). Additionally, secondary leukemias can develop in patients treated with cytotoxic agents such as radiation, alkylating agents, and epipodophyllotoxins.

Acute myeloid leukemia (AML) is a clonal hematologic disease resulting from acquired mutations in immature myeloid progenitor cells that block the differentiation of hematopoietic cells, thus leading to an accumulation of myeloid blasts [Passegue et al., Proc. Natl. Acad. Sci., (USA), 100 Supp. 1:11842-11849 (2003)]. Two classes of mutations, one impairing cell differentiation and the other conferring a survival and proliferative benefit, are known to cooperate to cause acute leukemias [Gilliland et al., Cancer Cell, 1:417-420 (2002)]. The French-American-British (FAB) classification is the standard system to classify the acute myeloid leukemias [Bennett et al., Ann. Intern. Med., 103:620-625 (1985)]. In individuals who achieve complete remission after chemotherapy, the remission duration is usually short. Overall, AML is a disease that is associated with a low rate of long term survival.

In chronic myelogenous leukemia, the Bcr-Abl fusion gene encodes a cytoplasmic protein with constitutive protein kinase activity, which leads to the activation of multiple downstream signaling cascades [Deininger et al., Blood, 96:3343-3356 (2000)]. Inhibition of the deregulated Abl kinase by the inhibitor imatinib has led to remarkable therapeutical success in treating this indication [Druker et al., N. Engl. J. Med., 344:1038-1042 (2001)]. The lymphocyte line also has acute and chronic leukemias (ALL and CLL, respectively).

The molecular pathogenesis of AML also involves the deregulation of several signal transduction pathways. STAT-related transcription factors are constitutively activated in acute myeloid leukemic blasts, and STAT3 activity may be associated with shorter disease-free survival [Gouilleux-Gruart et al., Blood, 87:1692-1697 (1996); Benekli et al., Blood, 99:252-257 (2002); Benekli et al., Blood, 101:2940-2954 (2003)]. Inappropriate mitogen-activated protein kinase (MAP-kinase) activation may also play a role in the leukemic transformation of myeloid cells [Milella et al., J. Clin. Invest., 108:851-859 (2001)]. Constitutive activation of NF-κB has also been described in leukemia and proposed as a major characteristic distinguishing AML stem cells from their normal counterparts [Guzman et al., Blood, 98:2301-2307 (2001); Guzman et al., Proc. Natl. Acad. Sci. (USA), 99:16220-16225 (2002)].

Lymphomas are cancers that originate in lymphocytes of lymphoid tissues including but not limited to the lymph nodes, bone marrow, spleen, and other organs of the immune system, and are characterized by uncontrolled increase in lymphocyte production. There are two basic categories of lymphomas, Hodgkin's lymphoma, which is marked by the presence of a hallmark cell type called the Reed-Sternberg cell, and non-Hodgkin's lymphomas, which includes a large, diverse group of lymphocytic cancers. The non-Hodgkin's lymphomas are generally classified according to lymphocyte cell lineage (including but not limited to B cells, T cells, and natural killer cells), and can be further divided into cancers that have an indolent (slowly progressing or low grade) course and those that have an aggressive (rapidly progressing or intermediate or high grade) course. Non-Hodgkin's lymphomas include but are not limited to B-cell lymphoma, Burkitt's lymphoma, diffuse cell lymphoma, follicular lymphoma, immunoblastic large cell lymphoma, lymphoblastic lymphoma, mantle cell lymphoma, mycosis fungoides, post-transplantation lymphoproliferative disorder, small non-cleaved cell lymphoma, and T-cell lymphoma.

Myeloproliferative disorders also involve excessive production of certain types of blood cells in the bone marrow. Myeloproliferative disorders include but are not limited to polycythemia vera, chronic idiopathic myelofibrosis, and essential thrombocythemia. In polycythemia vera, red blood cells are overproduced in the bone marrow and build up in the blood stream. In chronic idiopathic myelofibrosis, aberrant proliferation of myeloid progenitor-derived cells leads to fibrosis in the bone marrow and eventually bone marrow failure (i.e., an underproduction of myeloid progenitor-derived cells). In essential thrombocythemia, the number of platelets are overproduced, but other cells in the blood are normal.

Myelodysplastic syndromes, sometimes referred to as pre-leukemias or “smoldering” leukemias, are additional indications in which the bone marrow does not function normally, a so called “ineffective hematopoiesis.” Immature blast cells do not mature properly and become overproduced, leading to a lack of effective mature blood cells. A myelodysplastic syndrome may develop following treatment with drugs or radiation therapy for other diseases, or it may develop without any known cause. Myelodysplastic syndromes are classified based on the appearance of bone marrow and blood cells as imaged by microscope. Myelodysplastic syndromes include but are not limited to refractory anemia, refractory anemia with ringed sideroblasts, refractory anemia with excess blasts, and refractory anemia with excess blasts in transformation.

Plasma cell neoplasms including but not limited to myelomas are malignancies of bone marrow plasma cells that resemble leukemia. The malignant plasma cells, otherwise known as myeloma cells, accumulate in the bone marrow and, unlike typical leukemias, rarely enter the blood stream. This progressive accumulation of myeloma cells within the marrow disrupts normal bone marrow function (most commonly reflected by anemia), reduces white cell and platelet counts, causes damage to surrounding bone, and suppresses normal immune function (reflected by reduced levels of effective immunoglobulins and increased susceptibility to infection). Myeloma cells usually grow in the form of localized tumors (plasmacytomas). Such plasmacytomas can be single or multiple and confined within bone marrow and bone (medullary) or developed outside of bone in soft tissue (extramedullary plasmacytomas). When there are multiple plasmacytomas inside or outside bone, the indication is also called multiple myeloma.

Such indications are typically treated with one or more therapies including but not limited to surgery, radiation therapy, chemotherapy, immunotherapy, and bone marrow and/or stem cell transplantation.

Surgery involves the bulk removal of diseased tissue. While surgery can be effectively used to remove certain tumors, for example, breast, colon, and skin, it cannot be used to treat tumors located in areas that are inaccessible to surgeons. Additionally, surgery cannot typically be successfully used to treat non-localized cancerous indications including but not limited to leukemias and myelomas.

Radiation therapy involves using high-energy radiation from x-rays, gamma rays, neutrons, and other sources (“radiation”) to kill rapidly dividing cells such as cancerous cells and to shrink tumors. Radiation therapy is well known in the art [Hellman, Cancer: Principles and Practice of Oncology, 248-275, 4th ed., vol. 1 (1993)]. Radiation therapy may be administered from outside the body (“external-beam radiation therapy”). Alternatively, radiation therapy can be administered by placing radioactive materials capable of producing radiation in or near the tumor or in an area near the cancerous cells. Systemic radiation therapy employs radioactive substances including but not limited to radiolabeled monoclonal antibodies that can circulate throughout the body or localize to specific regions or organs of the body. Brachytherapy involves placing a radioactive “seed” in proximity to a tumor. Radiation therapy is non-specific and often causes damage to any exposed tissues. Additionally, radiation therapy frequently causes individuals to experience side effects (such as nausea, fatigue, low leukocyte counts, etc.) that can significantly affect their quality of life and influence their continued compliance with radiation treatment protocols.

Chemotherapy involves administering chemotherapeutic agents that often act by disrupting cell replication or cell metabolism (e.g., by disrupting DNA metabolism, DNA synthesis, DNA transcription, or microtubule spindle function, or by perturbing chromosomal structural integrity by way of introducing DNA lesions). Chemotherapeutics are frequently non-specific in that they affect normal healthy cells as well as tumor cells. The maintenance of DNA integrity is essential to cell viability in normal cells. Chemotherapeutic agents must be potent enough to kill cancerous cells without causing too much damage to normal cells. Therefore, anticancer drugs typically have very low therapeutic indices, i.e., the window between the effective dose and the excessively toxic dose can be extremely narrow because the drugs cause a high percentage of damage to normal cells as well as tumor cells. Additionally, chemotherapy-induced side effects significantly affect the quality of life of an individual in need of treatment, and therefore frequently influence the individual's continued compliance with chemotherapy treatment protocols.

In many indications involving aberrant proliferation of hematopoietic cells, there are two main treatment phases: remission induction and post-remission treatment. Post-remission treatment may be referred to as consolidation therapy. Less frequently, a third phase of treatment involving long-term, low-dose chemotherapy (maintenance therapy). Although maintenance therapy may reduce the likelihood of relapses, the general consensus is that this benefit is outweighed by the increased risk of treatment-related mortality when extended maintenance treatment is given.

Remission induction is achieved in most patients using two or more drugs in combination to clear all detectable cancerous cells from the blood and/or bone marrow. Remission induction is essentially standard for all patients except those with acute promyelocytic leukemia (APL), a subtype of the cancer acute myeloid leukemia (AML). Remission induction normally involves administration of the drug cytarabine, optionally in combination with an anthracycline (including but not limited to daunorubicin, mitoxantrone, or idarubicin). Sometimes a third drug, such as etoposide or thioguanine, is also administered. The intensity of treatment typically causes severe bone marrow suppression. Myeloid colony-stimulating factors (G-CSF and GM-CSF) can be administered to induce myeloid progenitor cell production and shorten the period of granulocytopenia following induction therapy. For acute promyelocytic leukemia, (M3 stage) tretinoin (all-trans-retinoic acid, ATRA) is used to induce terminal differentiation of the leukemic cells (i.e., to induce the proliferating, immature cells to differentiate into nonproliferating, specialized, mature cells).

The disappearance of detectable cancerous cells from the blood and bone marrow does not necessarily mean that all malignant cells in the body have been killed. Thus, additional treatment with the same, or similar drugs as used in remission induction at the same, or lower doses are often administered soon after completion of the remission induction phase. In some treatment protocols, consolidation therapy is intensified by using cytarabine.

Cellular immune deficiency and tumor-associated immune suppression are linked with various cancerous indications [Hadden, Int. Immunopharmacol. 3(8):1061-1071 (2003)]. Consequently, immunotherapeutic compositions comprising cytokines, growth factors, antigens, and/or antibodies have been proposed for treating cancerous indications [Hadden, supra; Cebon et al., Cancer Immun., 16(3):7-25 (2003)].

Chemotherapy and radiation therapy generally affect cells that divide rapidly, and are therefore used to treat cancer because cancer cells divide more often than most healthy cells. However, bone marrow cells also divide frequently, and high-dose treatments of chemotherapy and/or radiation therapy can severely damage or destroy the individual's bone marrow. Without healthy bone marrow, the individual is no longer able to produce blood cells needed to carry oxygen, defend against infection, and prevent bleeding. Bone marrow transplantation (BMT) and peripheral blood stem cell transplantation (PBSCT) are procedures for restoring stem cells that have been eradicated by high doses of chemotherapy and/or radiation therapy. In autologous transplants, individuals receive their own stem cells. In syngeneic transplants, individuals receive stem cells from an identical twin. In allogeneic transplants, individuals receive stem cells from someone other than themselves or an identical twin.

Other cancer therapies are also known. For example, photodynamic therapy (PDT) involves the administration of a photosensitizing compound or drug, typically orally, intravenously, or topically, that can be activated by an external light source to destroy a target tissue. The photosensitizing drug itself is harmless and rapidly leaves normal cells, but it remains in rapidly proliferating cells including but not limited to cancer cells for a longer time. Typically, a laser is then aimed at a tumor (or other cell mass), thereby activating the photosensitizing drug and killing the cells that have absorbed it. Photodynamic therapy is typically used to treat very small tumors in individuals.

Radiofrequency ablation is a minimally invasive treatment involving the insertion of a catheter device into a tumor. The catheter is guided by imaging techniques and includes an electrode capable of transmitting radiofrequency energy disposed along the catheter tip. Tissues in proximity to the catheter device tip are exposed to the radiofrequency energy and localized cytotoxicity results from the heating effect caused by the transmitted radiofrequency energy [Johnson et al., J. Endourol. 17(8):557-62 (2003); Chang, BioMed. Eng. Online, 2:12 (2003)]. Radiation frequency ablation is advantageous in that the catheter device can be inserted in surgically inaccessible tumors. Radiation frequency ablation is most frequently used to treat small tumors including cancers of the liver.

Additionally, anti-angiogenic therapies have been proposed for treatment of hematological cancers including but not limited to leukemia, multiple myeloma, and lymphomas [Moehler et al., Ann. Hematol. 80(12):695-705 (2001)]. Furthermore, angiogenesis appears to be important both in the pathogenesis of acute myelogenous leukemia (AML) and for the susceptibility of AML blasts to chemotherapy [Glenjen et al., Int J cancer. 101(1):86-94 (2002)]. Thus, inhibiting angiogenesis could constitute a strategy for treating AML [Hussong et al., Blood. 95(1):309-13 (2000)].

The methods of the invention relate to selectively inhibiting phosphoinositide 3-kinase delta (PI3Kδ) activity in hematopoietic cells. The following discussion relates to phosphoinositide 3-kinases (PI3Ks).

The phosphoinositide 3-kinase (PI3K) signaling pathway regulates many cellular processes in hematopoiesis including cell proliferation and survival [Bouscary et al., Blood, 101:3436-3443 (2003)]. PI3K is a major signaling pathway involved in mitogenesis [Cantley, Science, 296:1655-1657 (2002)] and the deregulation of this pathway in a wide range of human cancers has been described [Vivanco et al., Nat. Rev. Cancer, 2:489-501 (2002)]. Structurally, PI3Ks exist as heterodimeric complexes, consisting of a p110 catalytic subunit and a p55, p85, or p101 regulatory subunit. There are four different p110 catalytic subunits, which are classified as p110α, p110β, p110γ, and p110δ [Wymann et al., Biochim. Biophys. Acta, 1436:127-150 (1998); Vanhaesebroeck et al., Trends Biochem. Sci., 22:267-272(1997)]. Three class IA catalytic subunits, p110α, p110β, and p110δ are tightly associated with a regulatory p85 subunit that contains two Src-homology (SH2) domains having a high affinity for specific phosphorylated tyrosine residues in receptors and cytoplasmic signaling proteins [Hiles et al., Cell, 70:419-429 (1992)]. Among the p110 PI3K subunits, p110δ is known to be preferentially expressed in hematopoietic cells, and more specifically in leukocytes [Vanhaesebroeck et al., Proc. Natl. Acad. Sci. (USA), 94:4330-4335 (1997)].

PI3Ks catalyze the addition of a phosphate group to the inositol ring of phosphoinositides [Wymann et al., Biochim. Biophys. Acta, 1436:127-150 (1998)]. One target of these phosphorylated products is the serine/threonine protein kinase B (PKB or Akt). Akt subsequently phosphorylates several downstream targets, including the Bcl-2 family member Bad and caspase-9, thereby inhibiting their pro-apoptotic functions [Datta et al., Cell 91: 231-41, (1997); Cardone et al., Science 282: 1318-21, (1998)]. Akt has also been shown to phosphorylate the forkhead transcription factor FKHR (also referred to as FOXO3a) [Tang et al., J. Biol. Chem., 274:16741-6 (1999)]. In addition, many other members of the apoptotic machinery as well as transcription factors contain the Akt consensus phosphorylation site [Datta et al., supra].

The nonselective phosphoinositide 3-kinase (PI3K) inhibitors, LY294002 and wortmannin, have been shown to differentially effect the proliferation of normal hematopoietic progenitor cells relative to chronic myelogenous leukemic cells [Marley et al., Br. J. Haematol., 125(4):500-511 (2004)]. Additionally, the aforementioned nonselective inhibitors promote apoptosis in acute myeloid leukemic cells relative to normal hematopoietic progenitor cells [Zhao et al., Leukemia, 18(2):267-75 (2004)].

LY294002 and wortmannin do not distinguish among the four members of class I PI3Ks. For example, the IC₅₀ values of wortmannin against each of the various class I PI3Ks are in the range of 1-10 nM. Similarly, the IC₅₀ values for LY294002 against each of these PI3Ks is about 1 μM [Fruman et al., Ann. Rev. Biochem., 67:481-507 (1998)]. These inhibitors are not only nonselective with respect to class I PI3Ks, but are also potent inhibitors of other enzymes including but not limited to DNA dependent protein kinase, FRAP-mTOR, smooth muscle myosin light chain kinase, and casein kinase 2 [Hartley et al., Cell 82:849 (1995); Davies et al., Biochem. J. 351:95 (2000); Brunn et al., EMBO J. 15:5256 (1996)].

Because p110β, p110β, p110γ, and p110δ are expressed differentially by a wide variety of cell types, the administration of nonselective PI3K inhibitors such as LY294002 and wortmannin almost certainly will also affect cell types that may not be targeted for treatment. Therefore, the effective therapeutic dose of such nonselective inhibitors would be expected to clinically unusable because otherwise non-targeted cell types will likely be affected, especially when such nonselective inhibitors are combined with cytotoxic therapies including but not limited to chemotherapy, radiation therapy, photodynamic therapies, radiofrequency ablation, and/or anti-angiogenic therapies.

Therefore, important and significant goals are to develop and make available safer and more effective methods of treating and preventing indications involving aberrant proliferation of hematopoietic cells, and to provide cancer and other therapies that facilitate clinical management and continued compliance of the individual being treated with treatment protocols.

### SUMMARY OF THE INVENTION

The invention provides methods for treating and/or preventing aberrant proliferation of hematopoietic cells comprising selectively inhibiting phosphoinositide 3-kinase delta (PI3Kδ) activity in hematopoietic cells. In one aspect, the methods comprise administering an amount of a PI3Kδ selective inhibitor effective to inhibit PI3Kδ activity of hematopoietic cells. In another aspect, a PI3Kδ selective inhibitor is administered in an amount effective to inhibit Akt phosphorylation in hematopoietic cells. In an additional aspect, a PI3Kδ selective inhibitor is administered in an amount effective to inhibit FOXO3a phosphorylation in hematopoietic cells. In a further aspect, a PI3Kδ selective inhibitor is administered in an amount effective to inhibit GAB1 phosphorylation in hematopoietic cells. In a further aspect, a PI3Kδ selective inhibitor is administered in an amount effective to inhibit GAB2 phosphorylation in hematopoietic cells.

In one aspect, the methods are carried out ex vivo. In another aspect, the methods are carried out in vivo. The methods may generally be used to treat any indication involving aberrant proliferation of lymphoid and/or myeloid progenitor cells. In one aspect, the indication is selected from the group consisting of acute lymphoblastic leukemia; acute myeloid leukemia; chronic lymphocytic leukemia; chronic myelogenous leukemia; hairy cell leukemia; polycythemia vera; chronic idiopathic myelofibrosis; essential thrombocythemia; refractory anemia; refractory anemia with ringed sideroblasts; refractory anemia with excess blasts; refractory anemia with excess blasts in transformation; Hodgkin's lymphoma; B-cell lymphoma; Burkitt's lymphoma; diffuse cell lymphoma; follicular lymphoma; immunoblastic large cell lymphoma; lymphoblastic lymphoma; mantle cell lymphoma; mycosis fungoides; post-transplantation lymphoproliferative disorder; small non-cleaved cell lymphoma; T-cell lymphoma; and, plasma cell neoplasms. The methods are particularly effective when the PI3K pathway is constitutively activated in the hematopoietic cells.

The methods may further comprise administering a mammalian target of rapamycin (mTOR) inhibitor. In one aspect of this embodiment, the mTOR inhibitor is selected from the group consisting of rapamycin, FK506, cyclosporine A (CsA), and everolimus.

In another embodiment, the invention provides methods for treating and/or preventing leukemia comprising selectively inhibiting phosphoinositide 3-kinase delta (PI3Kδ) activity in leukemic cells. In one aspect, the methods comprise administering an amount of a PI3Kδ selective inhibitor effective to inhibit PI3Kδ activity of leukemic cells. In another aspect, a PI3Kδ selective inhibitor is administered in an amount effective to inhibit Akt phosphorylation in leukemic cells. In a further aspect, a PI3Kδ selective inhibitor is administered in an amount effective to inhibit FOXO3a phosphorylation in leukemic cells. In a further aspect, a PI3Kδ selective inhibitor is administered in an amount effective to inhibit GAB1 phosphorylation in leukemic cells. In a further aspect, a PI3Kδ selective inhibitor is administered in an amount effective to inhibit GAB2 phosphorylation in leukemic cells.

In one aspect, the methods are carried out ex vivo. In another aspect, the methods are carried out in vivo. The methods may generally be used to treat any leukemia involving aberrant proliferation of lymphoid and/or myeloid progenitor cells. In one aspect, the leukemia is selected from the group consisting of acute lymphoblastic leukemia; acute myeloid leukemia; chronic lymphocytic leukemia; chronic myelogenous leukemia; and, hairy cell leukemia. The methods are particularly effective when the PI3K pathway is constitutively activated in the leukemic cells.

The methods may further comprise administering a mammalian target of rapamycin (mTOR) inhibitor. In one aspect of this embodiment, the mTOR inhibitor is selected from the group consisting of rapamycin, FK506, cyclosporine A (CsA), and everolimus.

### DETAILED DESCPRIPTION

Hematopoietic cells typically differentiate into either lymphoid progenitor cells or myeloid progenitor cells, both of which ultimately differentiate into various mature cell types including but not limited to leukocytes. Aberrant proliferation of hematopoietic cells of one type often interferes with the production or survival of other hematopoietic cell types, which can result in compromised immunity, anemia, and/or thrombocytopenia. The methods of the invention treat and/or prevent aberrant proliferation of hematopoietic cells by inhibiting aberrant proliferation of hematopoietic cells.

The invention provides methods for treating and/or preventing aberrant proliferation of hematopoietic cells comprising selectively inhibiting phosphoinositide 3-kinase delta (PI3Kδ) activity in hematopoietic cells. Thus, the methods of the invention include treating and/or preventing aberrant proliferation of hematopoietic cells by inhibiting an upstream target in the pathway that selectively activates PI3Kδ. In one aspect of this embodiment, the methods comprise administering an amount of a PI3Kδ selective inhibitor effective to inhibit PI3Kδ activity of hematopoietic cells.

As used herein, the term “aberrant proliferation” means cell proliferation that deviates from the normal, proper, or expected course. For example, aberrant cell proliferation may include inappropriate proliferation of cells whose DNA or other cellular components have become damaged or defective. Aberrant cell proliferation may include cell proliferation whose characteristics are associated with an indication caused by, mediated by, or resulting in inappropriately high levels of cell division, inappropriately low levels of apoptosis, or both. Such indications may be characterized, for example, by single or multiple local abnormal proliferations of cells, groups of cells, or tissue(s), whether cancerous or non-cancerous, benign or malignant.

As used herein, the term “hematopoietic cells” generally refers to blood cells including but not limited to lymphoid progenitor cells, myeloid progenitor cells, natural killer cells, T cells, B cells, plasma cells, erythrocytes, megakaryocytes, monocytes, macrophages, and granulocytes such as neutrophils, eosinophils, and basophils.

As used herein, the term “selectively inhibiting phosphoinositide 3-kinase delta (PI3Kδ) activity” generally refers to inhibiting the activity of the PI3Kδ isozyme more effectively than other isozymes of the PI3K family. Similarly, the term “PI3Kδ selective inhibitor” generally refers to a compound that inhibits the activity of the PI3Kδ isozyme more effectively than other isozymes of the PI3K family. A PI3Kδ selective inhibitor compound is therefore more selective for PI3Kδ than conventional PI3K inhibitors such as wortmannin and LY294002, which are “nonselective PI3K inhibitors.”

As used herein, the term “amount effective” means a dosage sufficient to produce a desired or stated effect.

The methods of the invention may generally be used to treat and/or prevent indications involving aberrant proliferation of hematopoietic cells. Accordingly, the methods may be used to treat and/or prevent indication involving aberrant proliferation of lymphoid and/or myeloid progenitor cells including but not limited to leukemias such as acute lymphoblastic leukemia, acute myeloid leukemia; chronic lymphocytic leukemia, chronic myelogenous leukemia, and hairy cell leukemia; myeloproliferative disorders such as polycythemia vera, chronic idiopathic myelofibrosis, and essential thrombocythemia; myelodysplastic syndromes such as refractory anemia, refractory anemia with ringed sideroblasts, refractory anemia with excess blasts, and refractory anemia with excess blasts in transformation; lymphomas such as Hodgkin's lymphoma and non-Hodgkin's lymphomas such as B-cell lymphoma, Burkitt's lymphoma, diffuse cell lymphoma, follicular lymphoma, immunoblastic large cell lymphoma, lymphoblastic lymphoma, mantle cell lymphoma, mycosis fungoides, post-transplantation lymphoproliferative disorder, small non-cleaved cell lymphoma, and T-cell lymphoma; and, plasma cell neoplasms such as myelomas.

In another embodiment, the invention provides methods for treating and/or preventing leukemia comprising selectively inhibiting phosphoinositide 3-kinase delta (PI3Kδ) activity in leukemic cells. In one aspect of this embodiment, the methods comprise administering an amount of a PI3Kδ selective inhibitor effective to inhibit PI3Kδ activity of hematopoietic cells.

As used herein, the term “leukemia” generally refers to cancers that are characterized by an uncontrolled increase in the number of at least one leukocyte and/or leukocyte precursor in the blood and/or bone marrow. Leukemias including but not limited to acute lymphoblastic leukemia (ALL); acute myeloid leukemia (AML); chronic lymphocytic leukemia (CLL); chronic myelogenous leukemia (CML); and, hairy cell leukemia are contemplated. “Leukemic cells” typically comprise cells of the aforementioned leukemias.

The PI3K pathway is constitutively activated in the aberrantly proliferating hematopoietic cells. In one aspect of this embodiment, a higher level of phosphorylated Akt protein is present in untreated aberrantly proliferating hematopoietic cells relative to normal hematopoietic cells (i.e., non-aberrantly proliferating hematopoietic cells). In an additional aspect, a higher level of phosphorylated FOXO3a protein is present in untreated hematopoietic cells, and/or a higher level of phosphorylated GAB1 protein or phosphorylated GAB 2 protein is present in untreated hematopoietic cells, in each instance relative to normal hematopoietic cells.

Thus, in one aspect, the PI3Kδ selective inhibitor is administered in an amount effective to inhibit Akt phosphorylation in aberrantly proliferating hematopoietic cells. In another aspect, the PI3Kδ selective inhibitor is administered in an amount effective to inhibit FOXO3a phosphorylation in aberrantly proliferating hematopoietic cells. In a further aspect, the PI3Kδ selective inhibitor is administered in an amount effective to inhibit GAB1 phosphorylation and/or GAB2 phosphorylation in aberrantly proliferating hematopoietic cells.

In an additional embodiment, the methods of the invention further comprise administering a mammalian target of rapamycin (mTOR) inhibitor. In one aspect of this embodiment, the mTOR inhibitor is rapamycin. Other mTOR inhibitors that may be used include FK506, cyclosporine A (CsA), and everolimus.

As previously described, the term “PI3Kδ selective inhibitor” generally refers to a compound that inhibits the activity of the PI3Kδ isozyme more effectively than other isozymes of the PI3K family. The relative efficacies of compounds as inhibitors of an enzyme activity (or other biological activity) can be established by determining the concentrations at which each compound inhibits the activity to a predefined extent and then comparing the results. Typically, the preferred determination is the concentration that inhibits 50% of the activity in a biochemical assay, i.e., the 50% inhibitory concentration or “IC₅₀.” IC₅₀ determinations can be accomplished using conventional techniques known in the art. In general, an IC₅₀ can be determined by measuring the activity of a given enzyme in the presence of a range of concentrations of the inhibitor under study. The experimentally obtained values of enzyme activity then are plotted against the inhibitor concentrations used. The concentration of the inhibitor that shows 50% enzyme activity (as compared to the activity in the absence of any inhibitor) is taken as the IC₅₀ value. Analogously, other inhibitory concentrations can be defined through appropriate determinations of activity. For example, in some settings it can be desirable to establish a 90% inhibitory concentration, i.e., IC₉₀, etc.

Accordingly, a PI3Kδ selective inhibitor alternatively can be understood to refer to a compound that exhibits a 50% inhibitory concentration (IC₅₀) with respect to PI3Kδ that is at least 10-fold, in another aspect at least 20-fold, and in another aspect at least 30-fold, lower than the IC₅₀ value with respect to any or all of the other class I PI3K family members. In an alternative embodiment of the invention, the term PI3Kδ selective inhibitor can be understood to refer to a compound that exhibits an IC₅₀ with respect to PI3Kδ that is at least 50-fold, in another aspect at least 100-fold, in an additional aspect at least 200-fold, and in yet another aspect at least 500-fold, lower than the IC₅₀ with respect to any or all of the other PI3K class I family members. A PI3Kδ selective inhibitor is typically administered in an amount such that it selectively inhibits PI3Kδ activity, as described above.

Any selective inhibitor of PI3Kδ activity, including but not limited to small molecule inhibitors, peptide inhibitors, non-peptide inhibitors, naturally occurring inhibitors, and synthetic inhibitors, may be used in the methods. Suitable PI3Kδ selective inhibitors have been described in U.S. Patent Publication 2002/161014 to Sadhu et al., the entire disclosure of which is hereby incorporated herein by reference. Compounds that compete with a PI3Kδ selective inhibitor compound described herein for binding to PI3Kδ and selectively inhibit PI3Kδ are also contemplated for use in the methods of the invention. Methods of identifying compounds which competitively bind with PI3Kδ, with respect to the PI3Kδ selective inhibitor compounds specifically provided herein, are well known in the art [see, e.g., Coligan et al., Current Protocols in Protein Science, A.5A.15-20, vol. 3 (2002)]. In view of the above disclosures, therefore, PI3Kδ selective inhibitor embraces the specific PI3Kδ selective inhibitor compounds disclosed herein, compounds having similar inhibitory profiles, and compounds that compete with the such PI3Kδ selective inhibitor compounds for binding to PI3Kδ, and in each case, conjugates and derivatives thereof.

The methods of the invention may be applied to cell populations in vivo or ex vivo. “In vivo” means within a living individual, as within an animal or human. In this context, the methods of the invention may be used therapeutically in an individual, as described infra. The methods may also be used prophylactically including but not limited to when certain risk factors associated with a given indication treatable by the methods of the invention are present, particularly when two or more such risk factors are present. Many such risk factors are related to an individual's risk of relapse. Individuals having a high risk of relapse include but are not limited to individuals having chromosomal abnormalities involving chromosomes 3, 5, and/or 7. Other risk factors include but are not limited to the following: having a close relative who has been diagnosed with an indication involving aberrant proliferation of hematopoietic cells; having Down's syndrome or other disease caused by abnormal chromosomes; repeated or substantial exposure to benzene and/or other organic solvents; exposure to high doses of ionizing radiation; having received treatments comprising certain chemotherapeutic agents; exposure to diagnostic X-rays during pregnancy; infection with human T-cell leukemia virus; and, cigarette smoking and/or substantial exposure to smoke. Additional risk factors that may indicate that prophylactic treatment is warranted are known in the art and/or may be readily determined by the attending physician.

“Ex vivo” means outside of a living individual. Examples of ex vivo cell populations include in vitro cell cultures and biological samples including but not limited to fluid or tissue samples obtained from individuals. Such samples may be obtained by methods well known in the art. Exemplary biological fluid samples include blood, cerebrospinal fluid, urine, saliva. Exemplary tissue samples include tumors and biopsies thereof. In this context, the invention may be used for a variety of purposes, including therapeutic and experimental purposes. For example, the invention may be used ex vivo to determine the optimal schedule and/or dosing of administration of a PI3Kδ selective inhibitor for a given indication, cell type, individual, and other parameters. Information gleaned from such use may be used for experimental purposes or in the clinic to set protocols for in vivo treatment. Other ex vivo uses for which the invention may be suited are described below or will become apparent to those skilled in the art.

Animal models of some of the foregoing indications involving aberrant proliferation of hematopoietic cells treatable by the invention include for example: non-obese diabetic-severe combined immune deficient (NOD/scid) mice injected with human ALL cells (ALL model); athymic (rnu/rnu) nude rats injected with human ALL cells (e.g., HPB-ALL cells) (ALL model); NOD/scid mice injected with human CML cells (CML model); inbred Sprague-Dawley/Charles University Biology (SD/Cub) rats (spontaneous T-cell lymphoma/leukemia model); Emu-immediate-early response gene X-1 (IEX-1) mice (T-cell lymphoma model); rabbits injected with cynomogulus-Epstein Barr virus (T-cell lymphoma model); rabbits injected with Herpes virus papio (T-cell lymphoma model); transgenic mice expressing p210bcr/abl (founder mice, ALL model; progeny mice, CML model); NOD/scid/gammac null (NOG) mice injected with U266 cells (multiple myeloma model); and, C57B1/KaLwRij mice injected with 5T33 cells (multiple myeloma model).

It will be appreciated that the treatment methods of the invention are useful in the fields of human medicine and veterinary medicine. Thus, the individual to be treated may be a mammal, preferably human, or other animals. For veterinary purposes, individuals include but are not limited to farm animals including cows, sheep, pigs, horses, and goats; companion animals such as dogs and cats; exotic and/or zoo animals; laboratory animals including mice, rats, rabbits, guinea pigs, and hamsters; and poultry such as chickens, turkeys, ducks, and geese.

The methods of the invention may further comprise administration of radiation therapy. Radiation therapy is well known in the art [Hellman, Cancer: Principles and Practice of Oncology, 248-275, 4th ed., vol. 1 (1993)]. In one aspect, radiation therapy is administered from outside the body (“external-beam radiation therapy”). In another aspect, radiation therapy is administered by placing radioactive materials capable of producing radiation in or near the tumor or in an area near the cancerous cells. In the methods involving administration of radiation, external radiation is typically administered to an individual in an amount of about 1.8 Gy/day to about 3 Gy/day to a total dose of 30 to 70 Gy, with the total doses being administered over a period of about two to about seven weeks.

The methods in accordance with the invention may include administering a PI3Kδ selective inhibitor with one or more other agents that either enhance the activity of the inhibitor or compliment its activity or use in treatment. Such additional factors and/or agents may produce an augmented or even synergistic effect when administered with a PI3Kδ selective inhibitor, or minimize side effects.

In one embodiment, the methods of the invention may include administering formulations comprising a PI3Kδ selective inhibitor of the invention with a particular cytokine, lymphokine, other hematopoietic factor, thrombolytic or anti-thrombotic factor, or anti-inflammatory agent before, during, or after administration of the PI3Kδ selective inhibitor. One of ordinary skill can easily determine if a particular cytokine, lymphokine, hematopoietic factor, thrombolytic or anti-thrombotic factor, and/or anti-inflammatory agent enhances or compliments the activity or use of the PI3Kδ selective inhibitors in treatment.

More specifically, and without limitation, the methods of the invention may comprise administering a PI3Kδ selective inhibitor with one or more of TNF, IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IFN, G-CSF, Meg-CSF, GM-CSF, thrombopoietin, stem cell factor, and erythropoietin. Compositions in accordance with the invention may also include other known angiopoietins such as Ang-2, Ang-4, and Ang-Y, growth factors such as bone morphogenic protein-1, bone morphogenic protein-2, bone morphogenic protein-3, bone morphogenic protein-4, bone morphogenic protein-5, bone morphogenic protein-6, bone morphogenic protein-7, bone morphogenic protein-8, bone morphogenic protein-9, bone morphogenic protein-10, bone morphogenic protein-11, bone morphogenic protein-12, bone morphogenic protein-13, bone morphogenic protein-14, bone morphogenic protein-15, bone morphogenic protein receptor IA, bone morphogenic protein receptor IB, brain derived neurotrophic factor, ciliary neutrophic factor, ciliary neutrophic factor receptor α, cytokine-induced neutrophil chemotactic factor 1, cytokine-induced neutrophil chemotactic factor 2α, cytokine-induced neutrophil chemotactic factor 2β, β endothelial cell growth factor, endothelin 1, epidermal growth factor, epithelial-derived neutrophil attractant, fibroblast growth factor 4, fibroblast growth factor 5, fibroblast growth factor 6, fibroblast growth factor 7, fibroblast growth factor 8, fibroblast growth factor 8b, fibroblast growth factor 8c, fibroblast growth factor 9, fibroblast growth factor 10, fibroblast growth factor acidic, fibroblast growth factor basic, glial cell line-derived neutrophic factor receptor al, glial cell line-derived neutrophic factor receptor α2, growth related protein, growth related protein α, growth related protein β, growth related protein γ, heparin binding epidermal growth factor, hepatocyte growth factor, hepatocyte growth factor receptor, insulin-like growth factor I, insulin-like growth factor receptor, insulin-like growth factor II, insulin-like growth factor binding protein, keratinocyte growth factor, leukemia inhibitory factor, leukemia inhibitory factor receptor α, nerve growth factor, nerve growth factor receptor, neurotrophin-3, neurotrophin-4, placenta growth factor, placenta growth factor 2, platelet derived endothelial cell growth factor, platelet derived growth factor, platelet derived growth factor A chain, platelet derived growth factor AA, platelet derived growth factor AB, platelet derived growth factor B chain, platelet derived growth factor BB, platelet derived growth factor receptor α, platelet derived growth factor receptor β, pre-B cell growth stimulating factor, stem cell factor, stem cell factor receptor, transforming growth factor α, transforming growth factor β, transforming growth factor β1, transforming growth factor β1.2, transforming growth factor β2, transforming growth factor β3, transforming growth factor β5, latent transforming growth factor β1, transforming growth factor β binding protein 1, transforming growth factor β binding protein II, transforming growth factor β binding protein III, tumor necrosis factor receptor type I, tumor necrosis factor receptor type II, urokinase-type plasminogen activator receptor, and chimeric proteins and biologically or immunologically active fragments thereof.

Additionally, and without limitation, the methods of the invention may comprise administering a PI3Kδ selective inhibitor with one or more chemotherapeutic agents including but not limited to alkylating agents, intercalating agents, antimetabolites, natural products, biological response modifiers, miscellaneous agents, and hormones and antagonists. Alkylating agents for use in the inventive methods include but are not limited to nitrogen mustards such as mechlorethamine, cyclophosphamide, ifosfamide, melphalan and chlorambucil, nitrosoureas such as carmustine (BCNU), lomustine (CCNU) and semustine (methyl-CCNU), ethylenimine/methylmelamines such as triethylenemelamine (TEM), triethylene thiophosphoramide (thiotepa) and hexamethylmelamine (HMM, altretamine), alkyl sulfonates such as busulfan, and triazines such as dacarbazine (DTIC). Antimetabolites include but are not limited to folic acid analogs (including methotrexate, trimetrexate, and pemetrexed disodium), pyrimidine analogs (including 5-fluorouracil, fluorodeoxyuridine, gemcitabine, cytosine arabinoside (AraC, cytarabine), 5-azacytidine and 2,2*-difluorodeoxycytidine), and purine analogs (including 6-mercaptopurine, 6-thioguanine, azathioprine, 2′-deoxycoformycin (pentostatin), erythrohydroxynonyladenine (EHNA), fludarabine phosphate and 2-chlorodeoxyadenosine (cladribine, 2-CdA)). Intercalating agents for use in the inventive methods include but are not limited to ethidium bromide and acridine. Natural products for use in the inventive methods include but are not limited to anti-mitotic drugs such as paclitaxel, docetaxel, vinca alkaloids (including vinblastine (VLB), vincristine, vindesine and vinorelbine), taxotere, estramustine and estramustine phosphate. Additional natural products for use in the inventive methods include epipodophyllotoxins such as etoposide and teniposide, antibiotics such as actimomycin D, daunomycin (rubidomycin), doxorubicin, mitoxantrone, idarubicin, bleomycins, plicamycin (mithramycin), mitomycin C, dactinomycin and actinomycin D, and enzymes such as L-asparaginase. Biological response modifiers for use in the inventive methods include but are not limited to interferon-alpha, IL-2, G-CSF and GM-CSF. Miscellaneous agents for use in the inventive methods include but are not limited to platinum coordination complexes such as cisplatin and carboplatin, anthracenediones such as mitoxantrone, substituted ureas such as hydroxyurea, methylhydrazine derivatives such as N-methylhydrazine (MIH) and procarbazine, and adrenocortical suppressants such as mitotane (o,p*-DDD) and aminoglutethimide. Hormones and antagonists for use in the inventive methods include but are not limited to adrenocorticosteroids/antagonists such as prednisone, dexamethasone and aminoglutethimide, progestins such as hydroxyprogesterone caproate, medroxyprogesterone acetate and megestrol acetate, estrogens such as diethylstilbestrol and ethinyl estradiol, antiestrogens such as tamoxifen, androgens such as testosterone propionate and fluoxymesterone, antiandrogens such as flutamide, gonadotropin-releasing hormone analogs and leuprolide, and non-steroidal antiandrogens such as flutamide.

In one aspect, the chemotherapeutic is a DNA-damaging chemotherapeutic. Specific types of DNA-damaging chemotherapeutic agents contemplated for use in the inventive methods include, e.g., alkylating agents and intercalating agents.

The methods of the invention can also further comprise administering a PI3Kδ selective inhibitor in combination with a photodynamic therapy protocol. Typically, a photosensitizer is administered orally, intravenously, or topically, and then activated by an external light source. Photosensitizers for use in the methods of the invention include but are not limited to psoralens, lutetium texaphyrin (Lutex), benzoporphyrin derivatives (BPD) such as Verteporfin and Photofrin porfimer sodium (PH), phthalocyanines and derivatives thereof. Lasers are typically used to activate the photosensitizer. Light-emitting diodes (LEDs) and florescent light sources can also be used, but these do result in longer treatment times.

Additionally, and without limitation, the methods of the invention may comprise administering a PI3Kδ selective inhibitor at least one anti-angiogenic agent including but not limited to plasminogen fragments such as angiostatin and endostatin; angiostatic steroids such as squalamine; matrix metalloproteinase inhibitors such as Bay-129566; anti-vascular endothelial growth factor (anti-VEGF) isoform antibodies; anti-VEGF receptor antibodies; inhibitors that target VEGF isoforms and their receptors; inhibitors of growth factor (e.g., VEGF, PDGF, FGF) receptor tyrosine kinase catalytic activity such as SU11248; inhibitors of FGF production such as interferon alpha; inhibitors of methionine aminopeptidase-2 such as TNP-470; copper reduction therapies such as tetrathiomolybdate; inhibitors of FGF-triggered angiogenesis such as thalidomide and analogues thereof; platelet factor 4; and thrombospondin.

Additionally, the methods of the invention can further comprise bone marrow transplantation (BMT) and/or peripheral blood stem cell transplantation (PBSCT) procedures. The transplants may alternatively be autologous transplants, syngeneic transplants, or allogeneic transplants.

Methods of the invention contemplate use of PI3Kδ selective inhibitor compound having formula (I) or pharmaceutically acceptable salts and solvates thereof:

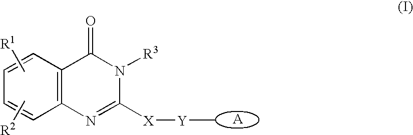

wherein A is an optionally substituted monocyclic or bicyclic ring system containing at least two nitrogen atoms, and at least one ring of the system is aromatic;

X is selected from the group consisting of C(R^{b})₂, CH₂CHR^{b}, and CH═C(R^{b});

Y is selected from the group consisting of null, S, SO, SO₂, NH, O, C(═O), OC(═O), C(═O)O, and NHC(═O)CH₂S;

R¹ and R², independently, are selected from the group consisting of hydrogen, C₁₋₆alkyl, aryl, heteroaryl, halo, NHC(═O)C₁₋₃alkyleneN(R^{a})₂, NO₂, OR^{a}, CF₃, OCF₃, N(R^{a})₂, CN, OC(═O)R^{a}, C(═O)R^{a}, C(═O)OR^{a}, arylOR^{b}, Het, NR^{a}C(═O)C₁₋₃alkyleneC(═O)OR^{a}, arylOC₁₋₃alkyleneN(R^{a})₂, arylOC(═O)R^{a}, C₁₋₄alkyleneC(═O)OR^{a}, OC₁₋₄alkyleneC(═O)OR^{a}, C₁₋₄alkyleneOC₁₋₄alkyleneC(═O)OR^{a}, C(═O)NR^{a}SO₂R^{a}, C₁₋₄alkyleneN(R^{a})₂, C₂₋₆alkenyleneN(R^{a})₂, C(═O)NR^{a}C₁₋₄alkyleneOR^{a}, C(═O)NR^{a}C₁₋₄alkyleneHet, OC₂₋₄alkyleneN(R^{a})₂, OC₁₋₄alkyleneCH(OR^{b})CH₂N(R^{a})₂, OC₁₋₄alkyleneHet, OC₂₋₄alkyleneOR^{a}, OC₂₋₄alkyleneNR^{a}C(═O)OR^{a}, NR^{a}C₁₋₄alkyleneN(R^{a})₂, NR^{a}C(═O)R^{a}, NR^{a}C(═O)N(R^{a})₂, N(SO₂C₁₋₄alkyl)₂, NR^{a}(SO₂C₁₋₄alkyl), SO₂N(R^{a})₂, OSO₂CF₃, C₁₋₃alkylenearyl, C₁₋₄alkyleneHet, C₆alkyleneOR^{b}, C₁₋₃alkyleneN(R^{a})₂, C(═O)N(R^{a})₂, NHC(═O)C₁₋₃alkylenearyl, C₃₋₈cycloalkyl, C₃₋₈heterocycloalkyl, arylOC₁₋₃alkyleneN(R^{a})₂, arylOC(═O)R^{b}, NHC(═O)C₁₋₃alkyleneC₃₋₈heterocycloalkyl, NHC(═O)C₁₋₃alkyleneHet, OC₁₋₄alkyleneOC₁₋₄alkyleneC(═O)OR^{b}, C(═O)C₁₋₄alkyleneHet, and NHC(═O)haloC₁₋₆alkyl;

or R¹ and R² are taken together to form a 3- or 4-membered alkylene or alkenylene chain component of a 5- or 6-membered ring, optionally containing at least one heteroatom;

R³ is selected from the group consisting of optionally substituted hydrogen, C₁₋₆alkyl, C₃₋₈cycloalkyl, C₃₋₈heterocycloalkyl, C₁₋₄alkylenecycloalkyl, C₂₋₆alkenyl, C₁₋₃alkylenearyl, arylC₁₋₃alkyl, C(═O)R^{a}, aryl, heteroaryl, C(═O)OR^{a}, C(═O)N(R^{a})₂, C(═S)N(R^{a})₂, SO₂R^{a}, SO₂N(R^{a})₂, S(═O)R^{a}, S(═O)N(R^{a})₂, C(═O)NR^{a}C₁₋₄alkyleneOR^{a}, C(═O)NR^{a}C₁₋₄alkyleneHet, C(═O)C₁₋₄alkylenearyl, C(═O)C₁₋₄alkyleneheteroaryl, C₁₋₄alkylenearyl optionally substituted with one or more of halo, SO₂N(R^{a})₂, N(R^{a})₂, C(═O)OR^{a}, NR^{a}SO₂CF₃, CN, NO₂, C(═O)R^{a}, OR^{a}, C₁₋₄alkyleneN(R^{a})₂, and OC₁₋₄alkyleneN(R^{a})₂, C₁₋₄alkyleneheteroaryl, C₁₋₄alkyleneHet, C₁₋₄alkyleneC(═O)C₁₋₄alkylenearyl, C₁₋₄alkyleneC(═O)C₁₋₄alkyleneheteroaryl, C₁₋₄alkyleneC(═O)Het, C₁₋₄alkyleneC(═O)N(R^{a})₂, C₁₋₄alkyleneOR^{a}, C₁₋₄alkyleneNR^{a}C(═O)R^{a}, C₁₋₄alkyleneOC₁₋₄alkyleneOR^{a}, C₁₋₄alkyleneN(R^{a})₂, C₁₋₄alkyleneC(═O)OR^{a}, and C₁₋₄alkyleneOC₁₋₄alkyleneC(═O)OR^{a};

R^{a} is selected from the group consisting of hydrogen, C₁₋₆alkyl, C₃₋₈cycloalkyl, C₃₋₈heterocycloalkyl, C₁₋₃alkyleneN(R^{c})₂, aryl, arylC₁₋₃alkyl, C₁₋₃alkylenearyl, heteroaryl, heteroarylC₁₋₃alkyl, and C₁₋₃alkyleneheteroaryl;

or two R^{a} groups are taken together to form a 5- or 6-membered ring, optionally containing at least one heteroatom;

R^{b} is selected from the group consisting of hydrogen, C₁₋₆alkyl, heteroC₁₋₃alkyl, C₁₋₃alkyleneheteroC₁₋₃alkyl, arylheteroC₁₋₃alkyl, aryl, heteroaryl, arylC₁₋₃alkyl, heteroarylC₁₋₃alkyl, C₁₋₃alkylenearyl, and C₁₋₃alkyleneheteroaryl;

R^{c} is selected from the group consisting of hydrogen, C₁₋₆alkyl, C₃₋₈cycloalkyl, aryl, and heteroaryl; and,

Het is a 5- or 6-membered heterocyclic ring, saturated or partially or fully unsaturated, containing at least one heteroatom selected from the group consisting of oxygen, nitrogen, and sulfur, and optionally substituted with C₁₋₄alkyl or C(═O)OR^{a}.

As used herein, the term “alkyl” is defined as straight chained and branched hydrocarbon groups containing the indicated number of carbon atoms, typically methyl, ethyl, and straight chain and branched propyl and butyl groups. The hydrocarbon group can contain up to 16 carbon atoms, for example, one to eight carbon atoms. The term “alkyl” includes “bridged alkyl,” i.e., a C₆₋C₁₆ bicyclic or polycyclic hydrocarbon group, for example, norbornyl, adamantyl, bicyclo[2.2.2]octyl, bicyclo[2.2.1]heptyl, bicyclo[3.2.1]octyl, or decahydronaphthyl. The term “cycloalkyl” is defined as a cyclic C₃-C₈ hydrocarbon group, e.g., cyclopropyl, cyclobutyl, cyclohexyl, and cyclopentyl.

The term “alkenyl” is defined identically as “alkyl,” except for containing a carbon-carbon double bond. “Cycloalkenyl” is defined similarly to cycloalkyl, except a carbon-carbon double bond is present in the ring.

The term “alkylene” is defined as an alkyl group having a substituent. For example, the term “C₁₋₃alkylenearyl” refers to an alkyl group containing one to three carbon atoms, and substituted with an aryl group.

The term “heteroC₁₋₃alkyl” is defined as a C₁₋₃alkyl group further containing a heteroatom selected from O, S, and NR^{a}, for example, —CH₂OCH₃ or —CH₂CH₂SCH₃. The term “arylheteroC₁₋₃alkyl” refers to an aryl group having a heteroC₁₋₃alkyl substituent.

The term “halo” or “halogen” is defined herein to include fluorine, bromine, chlorine, and iodine.

The term “aryl,” alone or in combination, is defined herein as a monocyclic or polycyclic aromatic group, e.g., phenyl or naphthyl. Unless otherwise indicated, an “aryl” group can be unsubstituted or substituted, for example, with one or more, and in particular one to three, halo, alkyl, phenyl, hydroxyalkyl, alkoxy, alkoxyalkyl, haloalkyl, nitro, and amino. Exemplary aryl groups include phenyl, naphthyl, biphenyl, tetrahydronaphthyl, chlorophenyl, fluorophenyl, aminophenyl, methylphenyl, methoxyphenyl, trifluoromethylphenyl, nitrophenyl, carboxyphenyl, and the like. The terms “arylC₁₋₁₃alkyl” and “heteroarylC₁₋₃ alkyl” are defined as an aryl or heteroaryl group having a C₁₋₃alkyl substituent.

The term “heteroaryl” is defined herein as a monocyclic or bicyclic ring system containing one or two aromatic rings and containing at least one nitrogen, oxygen, or sulfur atom in an aromatic ring, and which can be unsubstituted or substituted, for example, with one or more, and in particular one to three, substituents, like halo, alkyl, hydroxy, hydroxyalkyl, alkoxy, alkoxyalkyl, haloalkyl, nitro, and amino. Examples of heteroaryl groups include thienyl, furyl, pyridyl, oxazolyl, quinolyl, isoquinolyl, indolyl, triazolyl, isothiazolyl, isoxazolyl, imidizolyl, benzothiazolyl, pyrazinyl, pyrimidinyl, thiazolyl, and thiadiazolyl.

The term “Het” is defined as monocyclic, bicyclic, and tricyclic groups containing one or more heteroatoms selected from the group consisting of oxygen, nitrogen, and sulfur. A “Het” group also can contain an oxo group (═O) attached to the ring. Nonlimiting examples of Het groups include 1,3-dioxolane, 2-pyrazoline, pyrazolidine, pyrrolidine, piperazine, a pyrroline, 2H-pyran, 4H-pyran, morpholine, thiopholine, piperidine, 1,4-dithiane, and 1,4-dioxane.

Alternatively, the PI3Kδ selective inhibitor may be a compound having formula (II) or pharmaceutically acceptable salts and solvates thereof:

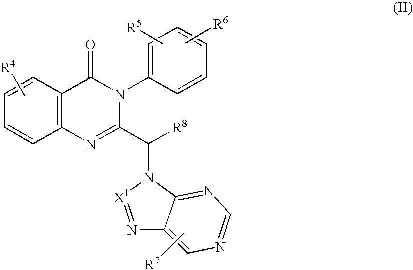

wherein R⁴, R⁵, R⁶, and R⁷, independently, are selected from the group consisting of hydrogen, C₁₋₆alkyl, aryl, heteroaryl, halo, NHC(═O)C₁₋₃alkyleneN(R^{a})₂, NO₂, OR^{a}, CF₃, OCF₃, N(R^{a})₂, CN, OC(═O)R^{a}, C(═O)R^{a}, C(═O)OR^{a}, arylOR^{b}, Het, NR^{a}C(═O)C₁₋₃alkyleneC(═O)OR^{a}, arylOC₁₋₃alkyleneN(R^{a})₂, arylOC(═O)R^{a}, C₁₋₄alkyleneC(═O)OR^{a}, OC₁₋₄alkyleneC(═O)OR^{a}, C₁₋₄alkyleneOC₁₋₄alkyleneC(═O)OR^{a}, C(═O)NR^{a}SO₂R^{a}, C₁₋₄alkyleneN(R^{a})₂, C₂₋₆alkenyleneN(R^{a})₂, C(═O)NR^{a}C₁₋₄alkyleneOR^{a}, C(═O)NR^{a}C₁₋₄alkyleneHet, OC₂₋₄alkyleneN(R a)₂, OC₁₋₄alkyleneCH(OR^{b})CH₂N(R^{a})₂, OC₁₋₄alkyleneHet, OC₂₋₄alkyleneOR^{a}, OC₂₋₄alkyleneNR^{a}C(═O)OR^{a}, NR^{a}C₁₋₄alkyleneN(R^{a})₂, NR^{a}C(═O)R^{a}, NR^{a}C(═O)N(R^{a})₂, N(SO₂C₁₋₄alkyl)₂, NR^{a}(SO₂C₁₋₄alkyl), SO₂N(R^{a})₂, OSO₂CF₃, C₁₋₃alkylenearyl, C₁₋₄alkyleneHet, C₁₋₆alkyleneOR^{b}, C₁₋₃alkyleneN(R^{a})₂, C(═O)N(R^{a})₂, NHC(═O)C₁₋₃alkylenearyl, C₃₋₈cycloalkyl, C₃₋₈heterocycloalkyl, arylOC₁₋₃alkyleneN(R^{a})₂, arylOC(═O)R^{b}, NHC(═O)C₁₋₃alkyleneC₃₋₈heterocycloalkyl, NHC(═O)C₁₋₃alkyleneHet, OC₁₋₄alkyleneOC₁₋₄alkyleneC(═O)OR^{b}, C(═O)C₁₋₄alkyleneHet, and NHC(═O)haloC₁₋₆alkyl;

R⁸ is selected from the group consisting of hydrogen, C₁₋₆alkyl, halo, CN, C(═O)R^{a}, and C(═O)OR^{a};

X¹ is selected from the group consisting of CH (i.e., a carbon atom having a hydrogen atom attached thereto) and nitrogen;

R^{a} is selected from the group consisting of hydrogen, C₁₋₆alkyl, C₃₋₈cycloalkyl, C₃₋₈heterocycloalkyl, C₁₋₃alkyleneN(R^{c})₂, aryl, arylC₁₋₃alkyl, C₁₋₃alkylenearyl, heteroaryl, heteroarylC₁₋₃alkyl, and C₁₋₃alkyleneheteroaryl;

or two R^{a} groups are taken together to form a 5- or 6-membered ring, optionally containing at least one heteroatom;

R^{c} is selected from the group consisting of hydrogen, C₁₋₆alkyl, C₃₋₈cycloalkyl, aryl, and heteroaryl; and,

Het is a 5- or 6-membered heterocyclic ring, saturated or partially or fully unsaturated, containing at least one heteroatom selected from the group consisting of oxygen, nitrogen, and sulfur, and optionally substituted with C₁₋₄alkyl or C(═O)OR^{a}.

The PI3Kδ selective inhibitor may also be a compound having formula (III) or pharmaceutically acceptable salts and solvates thereof:

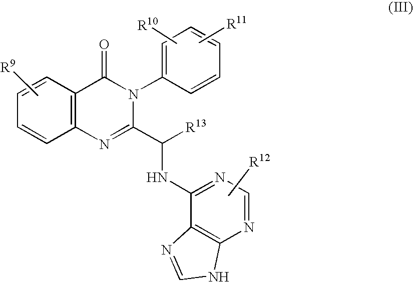

wherein R⁹, R¹⁰, R¹¹, and R¹², independently, are selected from the group consisting of hydrogen, amino, C₁₋₆alkyl, aryl, heteroaryl, halo, NHC(═O)C₁₋₃alkyleneN(R^{a})₂, NO₂, OR^{a}, CF₃, OCF₃, N(R^{a})₂, CN, OC(═O)R^{a}, C(═O)R^{a}, C(═O)OR^{a}, arylOR^{b}, Het, NR^{a}C(═O)C₁₋₃alkyleneC(═O)OR^{a}, arylOC₁₋₃alkyleneN(R^{a})₂, arylOC(═O)R^{a}, C₁₋₄alkyleneC(═O)OR^{a}, OC₁₋₄alkyleneC(═O)OR^{a}, C₁₋₄alkyleneOC₁₋₄alkyleneC(═O)OR^{a}, C(═O)NR^{a}SO₂R^{a}, C₁₋₄alkyleneN(R^{a})₂, C₂₋₆alkenyleneN(R^{a})₂, C(═O)NR^{a}C₁₋₄alkyleneOR^{a}, C(═O)NR^{a}C₁₋₄alkyleneHet, OC₂₋₄alkyleneN(R^{a})₂, OC₁₋₄alkyleneC H(OR^{b})CH₂N(R^{a})₂, OC₁₋₄alkyleneHet, OC₂₋₄alkyleneOR^{a}, OC₂₋₄alkyleneNR^{a}C(═O)OR^{a}, NR^{a}C₁₋₄alkyleneN(R^{a})₂, NR^{a}C(═O)R^{a}, NR^{a}C(═O)N(R^{a})₂, N(SO₂C₁₋₄alkyl)₂, NR^{a}(SO₂C₁₋₄alkyl), SO₂N(R^{a})₂, OSO₂CF₃, C₁₋₃alkylenearyl, C₁₋₄alkyleneHet, C₁₋₆alkyleneOR^{b}, C₁₋₃alkyleneN(R^{a})₂, C(═O)N(R^{a})₂, NHC(═O)C₁₋₃alkylenearyl, C₃₋₈cycloalkyl, C₃₋₈heterocycloalkyl, arylOC₁₋₃alkyleneN(R^{a})₂, arylOC(═O)R^{b}, NHC(═O)C₁₋₃alkyleneC₃₋₈heterocycloalkyl, NHC(═O)C₁₋₃alkyleneHet, OC₁₋₄alkyleneOC₁₋₄alkyleneC(═O)OR^{b}, C(═O)C₁₋₄alkyleneHet, and NHC(═O)haloC₁₋₆alkyl;

R¹³ is selected from the group consisting of hydrogen, C₁₋₆alkyl, halo, CN, C(═O)R^{a}, and C(═O)OR^{a};

R^{a} is selected from the group consisting of hydrogen, C₁₋₆alkyl, C₃₋₈cycloalkyl, C₃₋₈heterocycloalkyl, C₁₋₃alkyleneN(R^{c})₂, aryl, arylC₁₋₃alkyl, C₁₋₃alkylenearyl, heteroaryl, heteroarylC₁₋₃alkyl, and C₁₋₃alkyleneheteroaryl;

or two R^{a} groups are taken together to form a 5- or 6-membered ring, optionally containing at least one heteroatom;

R^{c} is selected from the group consisting of hydrogen, C₁₋₆alkyl, C₃₋₈cycloalkyl, aryl, and heteroaryl; and,

Het is a 5- or 6-membered heterocyclic ring, saturated or partially or fully unsaturated, containing at least one heteroatom selected from the group consisting of oxygen, nitrogen, and sulfur, and optionally substituted with C₁₋₄alkyl or C(═O)OR^{a}.

More specifically, representative PI3Kδ selective inhibitors in accordance with the foregoing chemical formulae include but are not limited to 2-(6-aminopurin-9-ylmethyl)-3-(2-chlorophenyl)-6,7-dimethoxy-3H-quinazolin-4-one; 2-(6-aminopurin-o-ylmethyl)-6-bromo-3-(2-chlorophenyl)-3H-quinazolin-4-one; 2-(6-aminopurin-o-ylmethyl)-3-(2-chlorophenyl)-7-fluoro-3H-quinazolin-4-one; 2-(6-aminopurin-9-ylmethyl)-6-chloro-3-(2-chlorophenyl)-3H-quinazolin-4-one; 2-(6-aminopurin-9-ylmethyl)-3-(2-chlorophenyl)-5-fluoro-3H-quinazolin-4-one; 2-(6-aminopurin-o-ylmethyl)-5-chloro-3-(2-chloro-phenyl)-3H-quinazolin-4-one; 2-(6-aminopurin-9-ylmethyl)-3-(2-chlorophenyl)-5-methyl-3H-quinazolin-4-one; 2-(6-aminopurin-9-ylmethyl)-8-chloro-3-(2-chlorophenyl)-3H-quinazolin-4-one; 2-(6-aminopurin-9-ylmethyl)-3-biphenyl-2-yl-5-chloro-3H-quinazolin-4-one; 5-chloro-2-(9H-purin-6-ylsulfanylmethyl)-3-o-tolyl-3H-quinazolin-4-one; 5-chloro-3-(2-fluorophenyl)-2-(9H-purin-6-yl-sulfanylmethyl)-3H-quinazolin-4-one; 2-(6-aminopurin-9-ylmethyl)-5-chloro-3-(2-fluorophenyl)-3H-quinazolin-4-one; 3-biphenyl-2-yl-5-chloro-2-(9H-purin-6-ylsulfanylmethyl)-3H-quinazolin-4-one; 5-chloro-3-(2-methoxyphenyl)-2-(9H-purin-6-yl-sulfanylmethyl)-3H-quinazolin-4-one; 3-(2-chlorophenyl)-5-fluoro-2-(9H-purin-6-yl-sulfanylmethyl)-3H-quinazolin-4-one; 3-(2-chlorophenyl)-6,7-dimethoxy-2-(9H-purin-6-yl-sulfanylmethyl)-3H-quinazolin-4-one; 6-bromo-3-(2-chlorophenyl)-2-(9H-purin-6-yl-sulfanylmethyl)-3H-quinazolin-4-one; 3-(2-chlorophenyl)-8-trifluoromethyl-2-(9H-purin-6-ylsulfanylmethyl)-3H-quinazolin-4-one; 3-(2-chlorophenyl)-2-(9H-purin-6-ylsulfanylmethyl)-3H-benzo[g]quinazolin-4-one; 6-chloro-3-(2-chlorophenyl)-2-(9H-purin-6-yl-sulfanylmethyl)-3H-quinazolin-4-one; 8-chloro-3-(2-chlorophenyl)-2-(9H-purin-6-yl-sulfanylmethyl)-3H-quinazolin-4-one; 3-(2-chlorophenyl)-7-fluoro-2-(9H-purin-6-yl-sulfanylmethyl)-3H-quinazolin-4-one; 3-(2-chlorophenyl)-7-nitro-2-(9H-purin-6-yl-sulfanylmethyl)-3H-quinazolin-4-one; 3-(2-chlorophenyl)-6-hydroxy-2-(9H-purin-6-yl-sulfanylmethyl)-3H-quinazolin-4-one; 5-chloro-3-(2-chlorophenyl)-2-(9H-purin-6-yl-sulfanylmethyl)-3H-quinazolin-4-one; 3-(2-chlorophenyl)-5-methyl-2-(9H-purin-6-yl-sulfanylmethyl)-3H-quinazolin-4-one; 3-(2-chlorophenyl)-6,7-difluoro-2-(9H-purin-6-yl-sulfanylmethyl)-3H-quinazolin-4-one; 3-(2-chlorophenyl)-6-fluoro-2-(9H-purin-6-yl-sulfanylmethyl)-3H-quinazolin-4-one; 2-(6-aminopurin-9-ylmethyl)-3-(2-isopropylphenyl)-5-methyl-3H-quinazolin-4-one; 2-(6-aminopurin-9-ylmethyl)-5-methyl-3-o-tolyl-3H-quinazolin-4-one; 3-(2-fluorophenyl)-5-methyl-2-(9H-purin-6-yl-sulfanylmethyl)-3H-quinazolin-4-one; 2-(6-aminopurin-9-ylmethyl)-5-chloro-3-o-tolyl-3H-quinazolin-4-one; 2-(6-aminopurin-9-ylmethyl)-5-chloro-3-(2-methoxy-phenyl)-3H-quinazolin-4-one; 2-(2-amino-9H-purin-6-ylsulfanylmethyl)-3-cyclopropyl-5-methyl-3H-quinazolin-4-one; 3-cyclopropylmethyl-5-methyl-2-(9H-purin-6-ylsulfanylmethyl)-3H-quinazolin-4-one; 2-(6-aminopurin-9-ylmethyl)-3-cyclopropylmethyl-5-methyl-3H-quinazolin-4-one; 2-(2-amino-9H-purin-6-ylsulfanylmethyl)-3-cyclopropylmethyl-5-methyl-3H-quinazolin-4-one; 5-methyl-3-phenethyl-2-(9H-purin-6-ylsulfanylmethyl)-3H-quinazolin-4-one; 2-(2-amino-9H-purin-6-ylsulfanylmethyl)-5-methyl-3-phenethyl-3H-quinazolin-4-one; 3-cyclopentyl-5-methyl-2-(9H-purin-6-ylsulfanylmethyl)-3H-quinazolin-4-one; 2-(6-aminopurin-9-ylmethyl)-3-cyclopentyl-5-methyl-3H-quinazolin-4-one; 3-(2-chloropyridin-3-yl)-5-methyl-2-(9H-purin-6-ylsulfanylmethyl)-3H-quinazolin-4-one; 2-(6-aminopurin-9-ylmethyl)-3-(2-chloropyridin-3-yl)-5-methyl-3H-quinazolin-4-one; 3-methyl-4-[5-methyl-4-oxo-2-(9H-purin-6-ylsulfanylmethyl)-4H-quinazolin-3-yl]-benzoic acid; 3-cyclopropyl-5-methyl-2-(9H-purin-6-ylsulfanylmethyl)-3H-quinazolin-4-one; 2-(6-aminopurin-9-ylmethyl)-3-cyclopropyl-5-methyl-3H-quinazolin-4-one; 5-methyl-3-(4-nitrobenzyl)-2-(9H-purin-6-ylsulfanylmethyl)-3H-quinazolin-4-one; 3-cyclohexyl-5-methyl-2-(9H-purin-6-ylsulfanylmethyl)-3H-quinazolin-4-one; 2-(6-aminopurin-9-ylmethyl)-3-cyclohexyl-5-methyl-3H-quinazolin-4-one; 2-(2-amino-9H-purin-6-ylsulfanylmethyl)-3-cyclo-hexyl-5-methyl-3H-quinazolin-4-one; 5-methyl-3-(E-2-phenylcyclopropyl)-2-(9H-purin-6-ylsulfanylmethyl)-3H-quinazolin-4-one; 3-(2-chlorophenyl)-5-fluoro-2-[(9H-purin-6-ylamino)methyl]-3H-quinazolin-4-one; 2-[(2-amino-9H-purin-6-ylamino)methyl]-3-(2-chlorophenyl)-5-fluoro-3H-quinazolin-4-one; 5-methyl-2-[(9H-purin-6-ylamino)methyl]-3-o-tolyl-3H-quinazolin-4-one; 2-[(2-amino-9H-purin-6-ylamino)methyl]-5-methyl-3-o-tolyl-3H-quinazolin-4-one; 2-[(2-fluoro-9H-purin-6-ylamino)methyl]-5-methyl-3-o-tolyl-3H-quinazolin-4-one; (2-chlorophenyl)-dimethylamino-(9H-purin-6-ylsulfanylmethyl)-3H-quinazolin-4-one; 5-(2-benzyloxyethoxy)-3-(2-chlorophenyl)-2-(9H-purin-6-ylsulfanylmethyl)-3H-quinazolin-4-one; 6-aminopurine-9-carboxylic acid 3-(2-chlorophenyl)-5-fluoro-4-oxo-3,4-dihydro-quinazolin-2-ylmethyl ester; N-[3-(2-chlorophenyl)-5-fluoro-4-oxo-3,4-dihydro-quinazolin-2-ylmethyl]-2-(9H-purin-6-ylsulfanyl)-acetamide; 2-[1-(2-fluoro-9H-purin-6-ylamino)ethyl]-5-methyl-3-o-tolyl-3H-quinazolin-4-one; 5-methyl-2-[1-(9H-purin-6-ylamino)ethyl]-3-o-tolyl-3H-quinazolin-4-one; 2-(6-dimethylaminopurin-9-ylmethyl)-5-methyl-3-o-tolyl-3H-quinazolin-4-one; 5-methyl-2-(2-methyl-6-oxo-1,6-dihydro-purin-7-ylmethyl)-3-o-tolyl-3H-quinazolin-4-one; 5-methyl-2-(2-methyl-6-oxo-1,6-dihydro-purin-9-ylmethyl)-3-o-tolyl-3H-quinazolin-4-one; 2-(amino-dimethylaminopurin-9-ylmethyl)-5-methyl-3-o-tolyl-3H-quinazolin-4-one; 2-(2-amino-9H-purin-6-ylsulfanylmethyl)-5-methyl-3-o-tolyl-3H-quinazolin-4-one; 2-(4-amino-1,3,5-triazin-2-ylsulfanylmethyl)-5-methyl-3-o-tolyl-3H-quinazolin-4-one; 5-methyl-2-(7-methyl-7H-purin-6-ylsulfanylmethyl)-3-o-tolyl-3H-quinazolin-4-one; 5-methyl-2-(2-oxo-1,2-dihydro-pyrimidin-4-ylsulfanylmethyl)-3-o-tolyl-3H-quinazolin-4-one; 5-methyl-2-purin-7-ylmethyl-3-o-tolyl-3H-quinazolin-4-one; 5-methyl-2-purin-9-ylmethyl-3-o-tolyl-3H-quinazolin-4-one; 5-methyl-2-(9-methyl-9H-purin-6-ylsulfanylmethyl)-3-o-tolyl-3H-quinazolin-4-one; 2-(2,6-diamino-pyrimidin-4-ylsulfanylmethyl)-5-methyl-3-o-tolyl-3H-quinazolin-4-one; 5-methyl-2-(5-methyl-[1,2,4]triazolo[1,5-a]pyrimidin-7-ylsulfanylmethyl)-3-o-tolyl-3H-quinazolin-4-one; 5-methyl-2-(2-methylsulfanyl-9H-purin-6-ylsulfanylmethyl)-3-o-tolyl-3H-quinazolin-4-one; 2-(2-hydroxy-9H-purin-6-ylsulfanylmethyl)-5-methyl-3-o-tolyl-3H-quinazolin-4-one; 5-methyl-2-(1-methyl-1H-imidazol-2-ylsulfanylmethyl)-3-o-tolyl-3H-quinazolin-4-one; 5-methyl-3-o-tolyl-2-(1H-[1,2,4]triazol-3-ylsulfanylmethyl)-3H-quinazolin-4-one; 2-(2-amino-6-chloro-purin-9-ylmethyl)-5-methyl-3-o-tolyl-3H-quinazolin-4-one; 2-(6-aminopurin-7-ylmethyl)-5-methyl-3-o-tolyl-3H-quinazolin-4-one; 2-(7-amino-1,2,3-triazolo[4,5-d]pyrimidin-3-yl-methyl)-5-methyl-3-o-tolyl-3H-quinazolin-4-one; 2-(7-amino-1,2,3-triazolo[4,5-d]pyrimidin-1-yl-methyl)-5-methyl-3-o-tolyl-3H-quinazolin-4-one; 2-(6-amino-9H-purin-2-ylsulfanylmethyl)-5-methyl-3-o-tolyl-3H-quinazolin-4-one; 2-(2-amino-6-ethylamino-pyrimidin-4-ylsulfanylmethyl)-5-methyl-3-o-tolyl-3H-quinazolin-4-one; 2-(3-amino-5-methylsulfanyl-1,2,4-triazol-1-yl-methyl)-5-methyl-3-o-tolyl-3H-quinazolin-4-one; 2-(5-amino-3-methylsulfanyl-1,2,4-triazol-1-ylmethyl)-5-methyl-3-o-tolyl-3H-quinazolin-4-one; 5-methyl-2-(6-methylaminopurin-9-ylmethyl)-3-o-tolyl-3H-quinazolin-4-one; 2-(6-benzylaminopurin-9-ylmethyl)-5-methyl-3-o-tolyl-3H-quinazolin-4-one; 2-(2,6-diaminopurin-9-ylmethyl)-5-methyl-3-o-tolyl-3H-quinazolin-4-one; 5-methyl-2-(9H-purin-6-ylsulfanylmethyl)-3-o-tolyl-3H-quinazolin-4-one; 3-isobutyl-5-methyl-2-(9H-purin-6-ylsulfanylmethyl)-3H-quinazolin-4-one; N-{2-[5-Methyl-4-oxo-2-(9H-purin-6-ylsulfanylmethyl)-4H-quinazolin-3-yl]-phenyl}-acetamide; 5-methyl-3-(E-2-methyl-cyclohexyl)-2-(9H-purin-6-ylsulfanylmethyl)-3H-quinazolin-4-one; 2-[5-methyl-4-oxo-2-(9H-purin-6-ylsulfanylmethyl)-4H-quinazolin-3-yl]-benzoic acid; 3-{2-[(2-dimethylaminoethyl)methylamino]phenyl}-5-methyl-2-(9H-purin-6-ylsulfanylmethyl)-3H-quin-azolin-4-one; 3-(2-chlorophenyl)-5-methoxy-2-(9H-purin-6-ylsulfanylmethyl)-3H-quinazolin-4-one; 3-(2-chlorophenyl)-5-(2-morpholin-4-yl-ethylamino)-2-(9H-purin-6-ylsulfanylmethyl)-3H-quinazolin-4-one; 3-benzyl-5-methoxy-2-(9H-purin-6-ylsulfanylmethyl)-3H-quinazolin-4-one; 2-(6-aminopurin-9-ylmethyl)-3-(2-benzyloxyphenyl)-5-methyl-3H-quinazolin-4-one; 2-(6-aminopurin-9-ylmethyl)-3-(2-hydroxyphenyl)-5-methyl-3H-quinazolin-4-one; 2-(1-(2-amino-9H-purin-6-ylamino)ethyl)-5-methyl-3-o-tolyl-3H-quinazolin-4-one; 5-methyl-2-[1-(9H-purin-6-ylamino)propyl]-3-o-tolyl-3H-quinazolin-4-one; 2-(1-(2-fluoro-9H-purin-6-ylamino)propyl)-5-methyl-3-o-tolyl-3H-quinazolin-4-one; 2-(1-(2-amino-9H-purin-6-ylamino)propyl)-5-methyl-3-o-tolyl-3H-quinazolin-4-one; 2-(2-benzyloxy-1-(9H-purin-6-ylamino)ethyl)-5-methyl-3-o-tolyl-3H-quinazolin-4-one; 2-(6-aminopurin-9-ylmethyl)-5-methyl-3-{2-(2-(1-methylpyrrolidin-2-yl)-ethoxy)-phenyl}-3H-quinazolin-4-one; 2-(6-aminopurin-9-ylmethyl)-3-(2-(3-dimethylamino-propoxy)-phenyl)-5-methyl-3H-quinazolin-4-one; 2-(6-aminopurin-9-ylmethyl)-5-methyl-3-(2-prop-2-ynyloxyphenyl)-3H-quinazolin-4-one; 2-{2-(1-(6-aminopurin-9-ylmethyl)-5-methyl-4-oxo-4H-quinazolin-3-yl]-phenoxy}-acetamide; 2-[(6-aminopurin-9-yl)methyl]-5-methyl-3-o-tolyl-3-hydroquinazolin-4-one; 3-(3,5-difluorophenyl)-5-methyl-2-[(purin-6-ylamino)methyl]-3-hydroquinazolin-4-one; 3-(2,6-dichlorophenyl)-5-methyl-2-[(purin-6-ylamino)methyl]-3-hydroquinazolin-4-one; 3-(2-Fluoro-phenyl)-2-[1-(2-fluoro-9H-purin-6-ylamino)-ethyl]-5-methyl-3-hydroquinazolin-4-one; 2-[1-(6-aminopurin-9-yl)ethyl]-3-(3,5-difluorophenyl)-5-methyl-3-hydroquinazolin-4-one; 2-[1-(7-Amino-[1,2,3]triazolo[4,5-d]pyrimidin-3-yl)-ethyl]-3-(3,5-difluoro-phenyl)-5-methyl-3H-quinazolin-4-one; 5-chloro-3-(3,5-difluoro-phenyl)-2-[1-(9H-purin-6-ylamino)-propyl]-3H-quinazolin-4-one; 3-phenyl-2-[1-(9H-purin-6-ylamino)-propyl]-3H-quinazolin-4-one; 5-fluoro-3-phenyl-2-[1-(9H-purin-6-ylamino)-propyl]-3H-quinazolin-4-one; 3-(2,6-difluoro-phenyl)-5-methyl-2-[1-(9H-purin-6-ylamino)-propyl]-3H-quinazolin-4-one; 6-fluoro-3-phenyl-2-[1-(9H-purin-6-ylamino)-ethyl]-3H-quinazolin-4-one; 3-(3,5-difluoro-phenyl)-5-methyl-2-[1-(9H-purin-6-ylamino)-ethyl]-3H-quinazolin-4-one; 5-fluoro-3-phenyl-2-[1-(9H-purin-6-ylamino)-ethyl]-3H-quinazolin-4-one; 3-(2,3-difluoro-phenyl)-5-methyl-2-[1-(9H-purin-6-ylamino)-ethyl]-3H-quinazolin-4-one; 5-methyl-3-phenyl-2-[1-(9H-purin-6-ylamino)-ethyl]-3H-quinazolin-4-one; 3-(3-chloro-phenyl)-5-methyl-2-[1-(9H-purin-6-ylamino)-ethyl]-3H-quinazolin-4-one; 5-methyl-3-phenyl-2-[(9H-purin-6-ylamino)-methyl]-3H-quinazolin-4-one; 2-[(2-amino-9H-purin-6-ylamino)-methyl]-3-(3,5-difluoro-phenyl)-5-methyl-3H-quinazolin-4-one; 3-{2-[(2-diethylamino-ethyl)-methyl-amino]-phenyl}-5-methyl-2-[(9H-purin-6-ylamino)-methyl]-3H-quinazolin-4-one; 5-chloro-3-(2-fluoro-phenyl)-2-[(9H-purin-6-ylamino)-methyl]-3H-quinazolin-4-one; 5-chloro-2-[(9H-purin-6-ylamino)-methyl]-3-o-tolyl-3H-quinazolin-4-one; 5-chloro-3-(2-chloro-phenyl)-2-[(9H-purin-6-ylamino)-methyl]-3H-quinazolin-4-one; 6-fluoro-3-(3-fluoro-phenyl)-2-[1-(9H-purin-6-ylamino)-ethyl]-3H-quinazolin-4-one; 2-[1-(2-amino-9H-purin-6-ylamino)-ethyl]-5-chloro-3-(3-fluoro-phenyl)-3H-quinazolin-4-one; 5-methyl-3-phenyl-2-[1-(9H-purin-6-ylamino)-propyl]-3H-quinazolin-4-one; 2-[1-(2-fluoro-9H-purin-6-ylamino)-ethyl]-5-methyl-3-phenyl-3H-quinazolin-4-one; 3-(2,6-difluoro-phenyl)-5-methyl-2-[1-(9H-purin-6-ylamino)-ethyl]-3H-quinazolin-4-one; 2-[1-(2-amino-9H-purin-6-ylamino)-ethyl]-3-(2,6-difluoro-phenyl)-5-methyl-3H-quinazolin-4-one; 3-(2,6-difluoro-phenyl)-2-[1-(2-fluoro-9H-purin-6-ylamino)-ethyl]-5-methyl-3H-quinazolin-4-one; 3-(2,6-difluoro-phenyl)-5-methyl-2-[1-(7H-pyrrolo[2,3-d]pyrimidin-4-ylamino)-ethyl]-3H-quinazolin-4-one; 2-[1-(2-amino-9H-purin-6-ylamino)-propyl]-5-methyl-3-phenyl-3H-quinazolin-4-one; 5-methyl-3-phenyl-2-[1-(7H-pyrrolo[2,3-d]pyrimidin-4-ylamino)-propyl]-3H-quinazolin-4-one; 2-[1-(2-fluoro-9h-purin-6-ylamino)-propyl]-5-methyl-3-phenyl-3h-quinazolin-4-one; 5-methyl-3-phenyl-2-[1-(9H-purin-6-ylamino)-ethyl]-3H-quinazolin-4-one; 2-[1-(2-amino-9H-purin-6-ylamino)-ethyl]-5-methyl-3-phenyl-3H-quinazolin-4-one; 2-[2-benzyloxy-1-(9H-purin-6-ylamino)-ethyl]-5-methyl-3-phenyl-3H-quinazolin-4-one; 2-[1-(2-amino-9H-purin-6-ylamino)-2-benzyloxy-ethyl]-5-methyl-3-phenyl-3H-quinazolin-4-one; 2-[2-benzyloxy-1-(7H-pyrrolo[2,3-d]pyrimidin-4-ylamino)-ethyl]-5-methyl-3-phenyl-3H-quinazolin-4-one; 2-[2-benzyloxy-1-(2-fluoro-9H-purin-6-ylamino)-ethyl]-5-methyl-3-phenyl-3H-quinazolin-4-one; 3-(4-fluoro-phenyl)-5-methyl-2-[1-(9H-purin-6-ylamino)-ethyl]-3H-quinazolin-4-one; 2-[1-(2-amino-9H-purin-6-ylamino)-ethyl]-3-(4-fluoro-phenyl)-5-methyl-3H-quinazolin-4-one; 3-(4-fluoro-phenyl)-2-[1-(2-fluoro-9H-purin-6-ylamino)-ethyl]-5-methyl-3H-quinazolin-4-one; 3-(4-fluoro-phenyl)-5-methyl-2-[1-(7H-pyrrolo[2,3-d]pyrimidin-4-ylamino)-ethyl]-3H-quinazolin-4-one; 5-methyl-3-phenyl-2-[1-(7H-pyrrolo[2,3-d]pyrimidin-4-ylamino)-ethyl]-3H-quinazolin-4-one; 3-(3-fluoro-phenyl)-5-methyl-2-[1-(9H-purin-6-ylamino)-ethyl]-3H-quinazolin-4-one; 2-[1-(2-amino-9H-purin-6-ylamino)-ethyl]-3-(3-fluoro-phenyl)-5-methyl-3H-quinazolin-4-one; 3-(3-fluoro-phenyl)-5-methyl-2-[1-(7H-pyrrolo[2,3-d]pyrimidin-4-ylamino)-ethyl]-3H-quinazolin-4-one; 5-methyl-3-phenyl-2-[1-(9H-purin-6-yl)-pyrrolidin-2-yl]-3H-quinazolin-4-one; 2-[2-hydroxy-1-(9H-purin-6-ylamino)-ethyl]-5-methyl-3-phenyl-3H-quinazolin-4-one; 5-methyl-3-phenyl-2-[phenyl-(9H-purin-6-ylamino)-methyl]-3H-quinazolin-4-one; 2-[(2-amino-9H-purin-6-ylamino)-phenyl-methyl]-5-methyl-3-phenyl-3H-quinazolin-4-one; 2-[(2-fluoro-9H-purin-6-ylamino)-phenyl-methyl]-5-methyl-3-phenyl-3H-quinazolin-4-one; 5-methyl-3-phenyl-2-[phenyl-(7H-pyrrolo[2,3-d]pyrimidin-4-ylamino)-methyl]-3H-quinazolin-4-one; 5-fluoro-3-phenyl-2-[1-(9H-purin-6-ylamino)-ethyl]-3H-quinazolin-4-one; 2-[1-(2-amino-9H-purin-6-ylamino)-ethyl]-5-fluoro-3-phenyl-3H-quinazolin-4-one; 2-[1-(2-amino-9H-purin-6-ylamino)-ethyl]-5-chloro-3-phenyl-3H-quinazolin-4-one; [5-(5-methyl-4-oxo-3-phenyl-3,4-dihydro-quinazolin-2-yl)-5-(9H-purin-6-ylamino)-pentyl]-carbamic acid benzyl ester; [5-(2-amino-9H-purin-6-ylamino)-5-(5-methyl-4-oxo-3-phenyl-3,4-dihydro-quinazolin-2-yl)-pentyl]-carbamic acid benzyl ester; [4-(5-methyl-4-oxo-3-phenyl-3,4-dihydro-quinazolin-2-yl)-4-(9H-purin-6-ylamino)-butyl]-carbamic acid benzyl ester; [4-(2-amino-9H-purin-6-ylamino)-4-(5-methyl-4-oxo-3-phenyl-3,4-dihydro-quinazolin-2-yl)-butyl]-carbamic acid benzyl ester; 3-phenyl-2-[1-(9H-purin-6-ylamino)-ethyl]-3H-quinazolin-4-one; 2-[5-amino-1-(9H-purin-6-ylamino)-pentyl]-5-methyl-3-phenyl-3H-quinazolin-4-one); 2-[5-amino-1-(2-amino-9H-purin-6-ylamino)-pentyl]-5-methyl-3-phenyl-3H-quinazolin-4-one; 2-[1-(2-amino-9H-purin-6-ylamino)-ethyl]-3-(2,6-Dimethyl-phenyl)-5-methyl-3H-quinazolin-4-one; 3-(2,6-dimethyl-phenyl)-5-methyl-2-[1-(9H-purin-6-ylamino)-ethyl]-3H-quinazolin-4-one; 5-morpholin-4-ylmethyl-3-phenyl-2-[1-(9H-purin-6-ylamino)-ethyl]-3H-quinazolin-4-one; 2-[1-(2-amino-9H-purin-6-ylamino)-ethyl]-5-morpholin-4ylmethyl-3-phenyl-3H-quinazolin-4-one; 2-[4-amino-1-(2-amino-9H-purin-6-ylamino)-butyl]-5-methyl-3-phenyl-3H-quinazolin-4-one; 6-fluoro-3-phenyl-2-[1-(9H-purin-6-ylamino)-ethyl]-3H-quinazolin-4-one; 2-[1-(2-amino-9H-purin-6-ylamino)-ethyl]-6-fluoro-3-phenyl-3H-quinazolin-4-one; 2-[2-tert-butoxy-1-(9H-purin-6-ylamino)-ethyl]-5-methyl-3-phenyl-3H-quinazolin-4-one; 3-(3-methyl-phenyl)-5-methyl-2-[1-(9H-purin-6-ylamino)-ethyl]-3H-quinazolin-4-one; 2-[1-(2-amino-9H-purin-6-ylamino)-ethyl]-3-(3-methyl-phenyl)-5-methyl-3H-quinazolin-4-one; 3-(3-chloro-phenyl)-5-methyl-2-[1-(9H-purin-6-ylamino)-ethyl]-3H-quinazolin-4-one; 2-[1-(2-amino-9H-purin-6-ylamino)-ethyl]-3-(3-chloro-phenyl)-5-methyl-3H-quinazolin-4-one; 2-[1-(2-amino-9H-purin-6-ylamino)-2-hydroxy-ethyl]-5-methyl-3-phenyl-3H-quinazolin-4-one; 2-[1-(2-amino-9H-purin-6-ylamino)-ethyl]-3-(3-fluoro-phenyl)-3H-quinazolin-4-one; 2-[1-(2-amino-9H-purin-6-ylamino)-ethyl]-3-(2,6-difluoro-phenyl)-3H-quinazolin-4-one; 2-[1-(2-amino-9H-purin-6-ylamino)-propyl]-5-fluoro-3-phenyl-3H-quinazolin-4-one; 5-chloro-3-(3-fluoro-phenyl)-2-[1-(9H-purin-6-ylamino)-ethyl]-3H-quinazolin-4-one; 2-[1-(2-amino-9H-purin-6-ylamino)-ethyl]-5-chloro-3-(3-fluoro-phenyl)-3H-quinazolin-4-one; 3-phenyl-2-[1-(9H-purin-6-ylamino)-ethyl]-5-trifluoromethyl-3H-quinazolin-4-one; 3-(2,6-difluoro-phenyl)-5-methyl-2-[1-(9H-purin-6-ylamino)-propyl]-3H-quinazolin-4-one; 3-(2,6-difluoro-phenyl)-5-methyl-2-[1-(9H-purin-6-ylamino)-ethyl]-3H-quinazolin-4-one; 2-[1-(2-amino-9H-purin-6-ylamino)-propyl]-3-(2,6-difluoro-phenyl)-5-methyl-3H-quinazolin-4-one; 2-[1-(2-amino-9H-purin-6-ylamino)-ethyl]-3-(2,6-difluoro-phenyl)-5-methyl-3H-quinazolin-4-one; 3-(3,5-dichloro-phenyl)-5-methyl-2-[1-(9H-purin-6-ylamino)-ethyl]-3H-quinazolin-4-one; 3-(2,6-dichloro-phenyl)-5-methyl-2-[1-(9H-purin-6-ylamino)-ethyl]-3H-quinazolin-4-one; 2-[1-(2-amino-9H-purin-6-ylamino)-ethyl]-3-(2,6-dichloro-phenyl)-5-methyl-3H-quinazolin-4-one; 5-chloro-3-phenyl-2-[1-(9H-purin-6-ylamino)-propyl]-3H-quinazolin-4-one; 2-[1-(2-amino-9H-purin-6-ylamino)-propyl]-5-chloro-3-phenyl-3H-quinazolin-4-one; 5-methyl-3-phenyl-2-[1-(9H-purin-6-ylamino)-butyl]-3H-quinazolin-4-one; 2-[1-(2-amino-9H-purin-6-ylamino)-butyl]-5-methyl-3-phenyl-3H-quinazolin-4-one; 2-[1-(2-amino-9H-purin-6-ylamino)-ethyl]-3-(3,5-dichloro-phenyl)-5-methyl-3H-quinazolin-4-one; 5-methyl-3-(3-morpholin-4-ylmethyl-phenyl)-2-[1-(9H-purin-6-ylamino)-ethyl]-3H-quinazolin-4-one; 2-[1-(2-amino-9H-purin-6-ylamino)-ethyl]-5-methyl-3-(3-morpholin-4-ylmethyl-phenyl)-3H-quinazolin-4-one; 2-[1-(5-bromo-7H-pyrrolo[2,3-d]pyrimidin-4-ylamino)-ethyl]-5-methyl-3-phenyl-3H-quinazolin-4-one; 5-methyl-2-[1-(5-methyl-7H-pyrrolo[2,3-d]pyrimidin-4-ylamino)-ethyl]-3-phenyl-3H-quinazolin-4-one; 2-[1-(5-fluoro-7H-pyrrolo[2,3-d]pyrimidin-4-ylamino)-ethyl]-5-methyl-3-phenyl-3H-quinazolin-4-one; 2-[2-hydroxy-1-(9H-purin-6-ylamino)-ethyl]-3-phenyl-3H-quinazolin-4-one; 3-(3,5-difluoro-phenyl)-5-methyl-2-[1-(9H-purin-6-ylamino)-propyl]-3H-quinazolin-4-one; 2-[1-(2-amino-9H-purin-6-ylamino)-propyl]-3-(3,5-difluoro-phenyl)-5-methyl-3H-quinazolin-4-one; 3-(3,5-difluoro-phenyl)-2-[1-(9H-purin-6-ylamino)-ethyl]-3H-quinazolin-4-one; 2-[1-(5-bromo-7H-pyrrolo[2,3-d]pyrimidin-4-ylamino)-ethyl]-3-(3-fluoro-phenyl)-5-methyl-3H-quinazolin-4-one; 3-(3-fluoro-phenyl)-5-methyl-2-[1-(5-methyl-7H-pyrrolo[2,3-d]pyrimidin-4-ylamino)-ethyl]-3H-quinazolin-4-one; 3-phenyl-2-[1-(9H-purin-6-ylamino)-propyl]-3H-quinazolin-4-one; 2-[1-(2-amino-9H-purin-6-ylamino)-ethyl]-3-(3,5-difluoro-phenyl)-3H-quinazolin-4-one; 2-[1-(2-amino-9H-purin-6-ylamino)-propyl]-3-phenyl-3H-quinazolin-4-one; 6,7-difluoro-3-phenyl-2-[1-(9H-purin-6-ylamino)-ethyl]-3H-quinazolin-4-one; 6-fluoro-3-(3-fluoro-phenyl)-2-[1-(9H-purin-6-ylamino)-ethyl]-3H-quinazolin-4-one; 2-[4-diethylamino-1-(9H-purin-6-ylamino)-butyl]-5-methyl-3-phenyl-3H-quinazolin-4-one; 5-fluoro-3-phenyl-2-[1-(9H-purin-6-ylamino)-propyl]-3H-quinazolin-4-one; 3-phenyl-2-[1-(9H-purin-6-ylamino)-ethyl]-3H-quinazolin-4-one; 6-fluoro-3-phenyl-2-[1-(9H-purin-6-ylamino)-ethyl]-3H-quinazolin-4-one; 3-(3,5-difluoro-phenyl)-5-methyl-2-[1-(9H-purin-6-ylamino)-ethyl]-3H-quinazolin-4-one; 5-fluoro-2-[1-(2-fluoro-9H-purin-6-ylamino)-ethyl]-3-phenyl-3H-quinazolin-4-one; 3-(3-fluoro-phenyl)-2-[1-(9H-purin-6-ylamino)-ethyl]-3H-quinazolin-4-one; 5-chloro-3-(3,5-difluoro-phenyl)-2-[1-(9H-purin-6-ylamino)-propyl]-3H-quinazolin-4-one; 3-(2,6-difluoro-phenyl)-5-methyl-2-[1-(9H-purin-6-ylamino)-ethyl]-3H-quinazolin-4-one; 3-(2,6-difluoro-phenyl)-2-[1-(9H-purin-6-ylamino)-ethyl]-3H-quinazolin-4-one; 5-Methyl-3-phenyl-2-[3,3,3-trifluoro-1-(9H-purin-6-ylamino)-propyl]-3H-quinazolin-4-one; 3-(3-hydroxy-phenyl)-5-methyl-2-[1-(9H-purin-6-ylamino)-ethyl]-3H-quinazolin-4-one; 3-(3-methoxy-phenyl)-5-methyl-2-{1-[9H-purin-6-ylamino]-ethyl}-3H-quinazolin-4-one; 3-[3-(2-dimethylamino-ethoxy)-phenyl]-5-methyl-2-{1-[9H-purin-6-ylamino]-ethyl}-3H-quinazolin-4-one; 3-(3-cyclopropylmethoxy-phenyl)-5-methyl-2-{1-[9H-purin-6-ylamino]-ethyl}-3H-quinazolin-4-one; 5-methyl-3-(3-prop-2-ynyloxy-phenyl)-2-{1-[9H-purin-6-ylamino]-ethyl}-3H-quinazolin-4-one; 2-{1-[2-amino-9H-purin-6-ylamino]ethyl}-3-(3-hydroxyphenyl)-5-methyl-3H-quinazolin-4-one; 2-{1-[2-amino-9H-purin-6-ylamino]ethyl}-3-(3-methoxyphenyl)-5-methyl-3H-quinazolin-4-one; 2-{1-[2-amino-9H-purin-6-ylamino]ethyl}-3-(3-cyclopropylmethoxy-phenyl)-5-methyl-3H-quinazolin-4-one; 2-{1-[2-amino-9H-purin-6-ylamino]ethyl}-5-methyl-3-(3-prop-2-ynyloxy-phenyl)-3H-quinazolin-4-one; 3-(3-ethynyl-phenyl)-5-methyl-2-[1-(9H-purin-6-ylamino)-ethyl]-3H-quinazolin-4-one; 3-{5-methyl-4-oxo-2-[1-(9H-purin-6-ylamino)-ethyl]-4H-quinazolin-3-yl}benzonitrile; 3-{5-methyl-4-oxo-2-{1-[9H-purin-6-ylamino)-ethyl]-4H-quinazolin-3-yl}-benzamide; 3-(3-acetyl-phenyl)-5-methyl-2-{1-[9H-purin-6-ylamino]-ethyl}-3H-quinazolin-4-one; 2-(3-(5-methyl-4-oxo-2-{1-[9H-purin-6-ylamino]ethyl}-4H-quinazolin-3-yl-phenoxy acetamide; 5-methyl-2-{1-[9H-purin-6-ylamino]-ethyl}-3-[3-(tetrahydropuran-4-yloxy)-phenyl]-3H-quinazolin-4-one; 3-[3-(2-methoxy-ethoxy)-phenyl]-5-methyl-2-[1-(9H-purin-6-ylamino)-ethyl]-3H-quinazolin-4-one; 6-fluoro-2-[1-(9H-purin-6-ylamino)ethyl]-3-[3-(tetrahydro-pyran-4-yloxy)-phenyl]-3H-quinazolin-4-one; 3-[3-(3-dimethylamino-propoxy)-phenyl]-5-methyl-2-[1-(9H-purin-6-ylamino)-ethyl]-3H-quinazolin-4-one; 2-[1-(2-amino-9H-purin-6-ylamino)-ethyl]-3-(3-ethynyl-phenyl)-5-methyl-3H-quinazolin-4-one; 3-{2-[1-(2-amino-9H-purin-6-ylamino)-ethyl]-5-methyl-4-oxo-4H-quinazolin-3-yl}-benzonitrile; 3-{2-[1-(2-amino-9H-purin-6-ylamino)-ethyl]-5-methyl-4-oxo-4H-quinazolin-3-yl}-benzamide; 3-{2-[1-(2-amino-9H-purin-6-ylamino)-ethyl]-5-methyl-4-oxo-4H-quinazolin-3-yl}-benzamide; 5-methyl-3-(3-morpholin-4-yl-phenyl)-2-[1-(9H-purin-6-ylamino)-ethyl]-3H-quinazolin-4-one; 2-[1-(2-amino-9H-purin-6-ylamino)-ethyl]-5-methyl-3-(3-morpholin-4-yl-phenyl)-3H-quinazolin-4-one; 2-[1-(2-amino-9H-purin-6-ylamino)-ethyl]-3-[3-(2-methoxy-ethoxy)-phenyl]-5-methyl-3H-quinazolin-4-one; 2-[1-(2-amino-9H-purin-6-ylamino)-ethyl]-3-[3-(2-dimethylamino-ethoxy)-phenyl]-5-methyl-3H-quinazolin-4-one; 2-[1-(2-amino-9H-purin-6-ylamino)-but-3-ynyl]-5-methyl-3-phenyl-3H-quinazolin-4-one; 2-[1-(2-amino-9H-purin-6-ylamino)-but-3-ynyl]-5-methyl-3-phenyl-3H-quinazolin-4-one; 5-chloro-3-(3,5-difluoro-phenyl)-2-[1-(9H-purin-6-ylamino)-ethyl]-3H-quinazolin-4-one; 2-[1-(2-amino-9H-purin-6-ylamino)-propyl]-5-chloro-3-(3,5-difluoro-phenyl)-3H-quinazolin-4-one; 2-[1-(2-amino-9H-purin-6-ylamino)-ethyl]-5-chloro-3-(3,5-difluoro-phenyl)-3H-quinazolin-4-one; 3-(3,5-difluoro-phenyl)-6-fluoro-2-[1-(9H-purin-6-ylamino)-ethyl]-3H-quinazolin-4-one; 5-chloro-3-(2,6-difluoro-phenyl)-2-[1-(9H-purin-6-ylamino)-propyl]-3H-quinazolin-4-one; 2-[1-(2-amino-9H-purin-6-ylamino)-propyl]-5-chloro-3-(2,6-difluoro-phenyl)-3H-quinazolin-4-one; 5-methyl-3-phenyl-2-[1-(9H-purin-6-yloxy)-ethyl]-3H-quinazolin-4-one; and mixtures thereof.

Where a stereocenter is present, the methods can be practiced using a racemic mixture of the compounds or a specific enantiomer. In preferred embodiments where a stereocenter is present, the S-enantiomer of the above compounds is utilized. However, the methods of the invention include administration of all possible stereoisomers and geometric isomers of the aforementioned compounds.

Additionally, the methods include administration of PI3Kδ selective inhibitors comprising an arylmorpholine moiety [Knight et al., Bioorganic & Medicinal Chemistry, 12:4749-4759 (2004)]. Representative PI3Kδ selective inhibitors include but are not limited to 2-morpholin-4-yl-8-o-tolyloxy-1H-quinolin-4-one; 9-bromo-7-methyl-2-morpholin-4-yl-pyrido(1,2-a)-pyrimidin-4-one; 9-benzylamino-7-methyl-2-morpholin-4-yl-pyrido-(1,2 a)pyrimidin-4-one; 9-(3-amino-phenyl)-7-methyl-2-morpholin-4-yl-pyrido[1,2-a]pyrimidin-4-one; 9-(2-methoxy-phenylamino)-7-methyl-2-morpholin-4-yl-pyrido(1,2-a)pyrimidin-4-one; 7-methyl-2-morpholin-4-yl-9-o-tolylamino-pyri-do(1,2-a)pyrimidin-4-one; 9-(3,4-dimethyl-phenylamino)-7-methyl-2-morph-olin-4-yl-pyrido(1,2-a)pyrimidin-4-one; 7-methyl-9-(3-methyl-benzylamino)-2-morpholin-4-yl-pyrido(1,2-a)pyrimidin-4-one; 9-(2,3-dimethyl-phenylamino)-7-methyl-2-morpholin-4-yl-pyrido(1,2-a)pyrimidin-4-one; 7-methyl-9-(2-methyl-benzylamino)-2-morpholin-4-yl-pyrido(1,2-a) pyrimidin-4-one; 5-morpholin-4-yl-2-nitro-phenylamine; 1-(2-hydroxy-4-morpholin-4-yl-phenyl)-phenyl-methanone; and, 2-chloro-1-(2-hydroxy-4-morpholin-4-yl-phenyl)-ethanone.

“Pharmaceutically acceptable salts” means any salts that are physiologically acceptable insofar as they are compatible with other ingredients of the formulation and not deleterious to the recipient thereof. Some specific preferred examples are: acetate, trifluoroacetate, hydrochloride, hydrobromide, sulfate, citrate, tartrate, glycolate, oxalate.

Administration of prodrugs is also contemplated. The term “prodrug” as used herein refers to compounds that are rapidly transformed in vivo to a more pharmacologically active compound. Prodrug design is discussed generally in Hardma et al. (Eds.), Goodman and Gilman's The Pharmacological Basis of Therapeutics, 9th ed., pp. 11-16 (1996). A thorough discussion is provided in Higuchi et al., Prodrugs as Novel Delivery Systems, Vol. 14, ASCD Symposium Series, and in Roche (ed.), Bioreversible Carriers in Drug Design, American Pharmaceutical Association and Pergamon Press (1987).

To illustrate, prodrugs can be converted into a pharmacologically active form through hydrolysis of, for example, an ester or amide linkage, thereby introducing or exposing a functional group on the resultant product. The prodrugs can be designed to react with an endogenous compound to form a water-soluble conjugate that further enhances the pharmacological properties of the compound, for example, increased circulatory half-life. Alternatively, prodrugs can be designed to undergo covalent modification on a functional group with, for example, glucuronic acid, sulfate, glutathione, amino acids, or acetate. The resulting conjugate can be inactivated and excreted in the urine, or rendered more potent than the parent compound. High molecular weight conjugates also can be excreted into the bile, subjected to enzymatic cleavage, and released back into the circulation, thereby effectively increasing the biological half-life of the originally administered compound.

Additionally, compounds that selectively negatively regulate p110δ mRNA expression more effectively than they do other isozymes of the PI3K family, and that possess acceptable pharmacological properties are contemplated for use as PI3Kδ selective inhibitors in the methods of the invention. Polynucleotides encoding human p110δ are disclosed, for example, in Genbank Accession Nos. AR255866, NM 005026, U86453, U57843 and Y10055, the entire disclosures of which are incorporated herein by reference [see also, Vanhaesebroeck et al., Proc. Natl. Acad. Sci., 94:4330-4335 (1997), the entire disclosure of which is incorporated herein by reference]. Representative polynucleotides encoding mouse p110δ are disclosed, for example, in Genbank Accession Nos. BC035203, AK040867, U86587, and NM_{—}008840, and a polynucleotide encoding rat p110δ is disclosed in Genbank Accession No. XM_{—}345606, in each case the entire disclosures of which are incorporated herein by reference.

In one embodiment, the invention provides methods using antisense oligonucleotides which negatively regulate p110δ expression via hybridization to messenger RNA (mRNA) encoding p110δ. In one aspect, antisense oligonucleotides at least 5 to about 50 nucleotides in length, including all lengths (measured in number of nucleotides) in between, which specifically hybridize to mRNA encoding p110δ and inhibit mRNA expression, and as a result p110δ protein expression, are contemplated for use in the methods of the invention. Antisense oligonucleotides include those comprising modified internucleotide linkages and/or those comprising modified nucleotides which are known in the art to improve stability of the oligonucleotide, i.e., make the oligonucleotide more resistant to nuclease degradation, particularly in vivo. It is understood in the art that, while antisense oligonucleotides that are perfectly complementary to a region in the target polynucleotide possess the highest degree of specific inhibition, antisense oligonucleotides that are not perfectly complementary, i.e., those which include a limited number of mismatches with respect to a region in the target polynucleotide, also retain high degrees of hybridization specificity and therefore also can inhibit expression of the target mRNA. Accordingly, the invention contemplates methods using antisense oligonucleotides that are perfectly complementary to a target region in a polynucleotide encoding p110δ, as well as methods that utilize antisense oligonucleotides that are not perfectly complementary (i.e., include mismatches) to a target region in the target polynucleotide to the extent that the mismatches do not preclude specific hybridization to the target region in the target polynucleotide. Preparation and use of antisense compounds is described, for example, in U.S. Pat. No. 6,277,981, the entire disclosure of which is incorporated herein by reference [see also, Gibson (Ed.), Antisense and Ribozyme Methodology, (1997), the entire disclosure of which is incorporated herein by reference].

The invention further contemplates methods utilizing ribozyme inhibitors which, as is known in the art, include a nucleotide region which specifically hybridizes to a target polynucleotide and an enzymatic moiety that digests the target polynucleotide. Specificity of ribozyme inhibition is related to the length the antisense region and the degree of complementarity of the antisense region to the target region in the target polynucleotide. The methods of the invention therefore contemplate ribozyme inhibitors comprising antisense regions from 5 to about 50 nucleotides in length, including all nucleotide lengths in between, that are perfectly complementary, as well as antisense regions that include mismatches to the extent that the mismatches do not preclude specific hybridization to the target region in the target p110δ-encoding polynucleotide. Ribozymes useful in methods of the invention include those comprising modified internucleotide linkages and/or those comprising modified nucleotides which are known in the art to improve stability of the oligonucleotide, i.e., make the oligonucleotide more resistant to nuclease degradation, particularly in vivo, to the extent that the modifications do not alter the ability of the ribozyme to specifically hybridize to the target region or diminish enzymatic activity of the molecule. Because ribozymes are enzymatic, a single molecule is able to direct digestion of multiple target molecules thereby offering the advantage of being effective at lower concentrations than non-enzymatic antisense oligonucleotides. Preparation and use of ribozyme technology is described in U.S. Pat. Nos. 6,696,250, 6,410,224, 5,225,347, the entire disclosures of which are incorporated herein by reference.

The invention also contemplates use of methods in which RNAi technology is utilized for inhibiting p110δ expression. In one aspect, the invention provides double-stranded RNA (dsRNA) wherein one strand is complementary to a target region in a target p110δ-encoding polynucleotide. In general, dsRNA molecules of this type are less than 30 nucleotides in length and referred to in the art as short interfering RNA (siRNA). The invention also contemplates, however, use of dsRNA molecules longer than 30 nucleotides in length, and in certain aspects of the invention, these longer dsRNA molecules can be about 30 nucleotides in length up to 200 nucleotides in length and longer, and including all length dsRNA molecules in between. As with other RNA inhibitors, complementarity of one strand in the dsRNA molecule can be a perfect match with the target region in the target polynucleotide, or may include mismatches to the extent that the mismatches do not preclude specific hybridization to the target region in the target p110δ-encoding polynucleotide. As with other RNA inhibition technologies, dsRNA molecules include those comprising modified internucleotide linkages and/or those comprising modified nucleotides which are known in the art to improve stability of the oligonucleotide, i.e., make the oligonucleotide more resistant to nuclease degradation, particularly in vivo. Preparation and use of RNAi compounds is described in U.S. Patent Application No. 20040023390, the entire disclosure of which is incorporated herein by reference.

The invention further contemplates methods wherein inhibition of p110δ is effected using RNA lasso technology. Circular RNA lasso inhibitors are highly structured molecules that are inherently more resistant to degradation and therefore do not, in general, include or require modified internucleotide linkage or modified nucleotides. The circular lasso structure includes a region that is capable of hybridizing to a target region in a target polynucleotide, the hybridizing region in the lasso being of a length typical for other RNA inhibiting technologies. As with other RNA inhibiting technologies, the hybridizing region in the lasso may be a perfect match with the target region in the target polynucleotide, or may include mismatches to the extent that the mismatches do not preclude specific hybridization to the target region in the target p110δ-encoding polynucleotide. Because RNA lassos are circular and form tight topological linkage with the target region, inhibitors of this type are generally not displaced by helicase action unlike typical antisense oligonucleotides, and therefore can be utilized as dosages lower than typical antisense oligonucleotides. Preparation and use of RNA lassos is described in U.S. Pat. No. 6,369,038, the entire disclosure of which is incorporated herein by reference.

The inhibitors of the invention may be covalently or noncovalently associated with a carrier molecule including but not limited to a linear polymer (e.g., polyethylene glycol, polylysine, dextran, etc.), a branched-chain polymer (see U.S. Pat. Nos. 4,289,872 and 5,229,490; PCT Publication No. WO 93/21259), a lipid, a cholesterol group (such as a steroid), or a carbohydrate or oligosaccharide. Specific examples of carriers for use in the pharmaceutical compositions of the invention include carbohydrate-based polymers such as trehalose, mannitol, xylitol, sucrose, lactose, sorbitol, dextrans such as cyclodextran, cellulose, and cellulose derivatives. Also, the use of liposomes, microcapsules or microspheres, inclusion complexes, or other types of carriers is contemplated.

Other carriers include one or more water soluble polymer attachments such as polyoxyethylene glycol, or polypropylene glycol as described U.S. Pat. Nos. 4,640,835, 4,496,689, 4,301,144, 4,670,417, 4,791,192 and 4,179,337. Still other useful carrier polymers known in the art include monomethoxy-polyethylene glycol, poly-(N-vinyl pyrrolidone)-polyethylene glycol, propylene glycol homopolymers, a polypropylene oxide/ethylene oxide co-polymer, polyoxyethylated polyols (e.g., glycerol) and polyvinyl alcohol, as well as mixtures of these polymers.

Derivatization with bifunctional agents is useful for cross-linking a compound of the invention to a support matrix or to a carrier. One such carrier is polyethylene glycol (PEG). The PEG group may be of any convenient molecular weight and may be straight chain or branched. The average molecular weight of the PEG can range from about 2 kDa to about 100 kDa, in another aspect from about 5 kDa to about 50 kDa, and in a further aspect from about 5 kDa to about 10 kDa. The PEG groups will generally be attached to the compounds of the invention via acylation, reductive alkylation, Michael addition, thiol alkylation or other chemoselective conjugation/ligation methods through a reactive group on the PEG moiety (e.g., an aldehyde, amino, ester, thiol, ci-haloacetyl, maleimido or hydrazino group) to a reactive group on the target inhibitor compound (e.g., an aldehyde, amino, ester, thiol, α-haloacetyl, maleimido or hydrazino group). Cross-linking agents can include, e.g., esters with 4-azidosalicylic acid, homobifunctional imidoesters, including disuccinimidyl esters such as 3,3′-dithiobis (succinimidylpropionate), and bifunctional maleimides such as bis-N-maleimido-1,8-octane. Derivatizing agents such as methyl-3-[(p-azidophenyl)dithio]propioimidate yield photoactivatable intermediates that are capable of forming crosslinks in the presence of light. Alternatively, reactive water-insoluble matrices such as cyanogen bromide-activated carbohydrates and the reactive substrates described in U.S. Pat. Nos. 3,969,287; 3,691,016; 4,195,128; 4,247,642; 4,229,537; and 4,330,440 may be employed for inhibitor immobilization.

The pharmaceutical compositions of the invention may also include compounds derivatized to include one or more antibody Fc regions. Fc regions of antibodies comprise monomeric polypeptides that may be in dimeric or multimeric forms linked by disulfide bonds or by non-covalent association. The number of intermolecular disulfide bonds between monomeric subunits of Fc molecules can be from one to four depending on the class (e.g., IgG, IgA, IgE) or subclass (e.g., IgG1, IgG2, IgG3, IgA1, IgGA2) of antibody from which the Fc region is derived. The term “Fc” as used herein is generic to the monomeric, dimeric, and multimeric forms of Fc molecules, with the Fc region being a wild type structure or a derivatized structure. The pharmaceutical compositions of the invention may also include the salvage receptor binding domain of an Fc molecule as described in WO 96/32478, as well as other Fc molecules described in WO 97/34631.

Such derivatized moieties preferably improve one or more characteristics of the inhibitor compounds of the invention, including for example, biological activity, solubility, absorption, biological half life, and the like. Alternatively, derivatized moieties result in compounds that have the same, or essentially the same, characteristics and/or properties of the compound that is not derivatized. The moieties may alternatively eliminate or attenuate any undesirable side effect of the compounds and the like.

Methods include administration of an inhibitor to an individual in need, by itself, or in combination as described herein, and in each case optionally including one or more suitable diluents, fillers, salts, disintegrants, binders, lubricants, glidants, wetting agents, controlled release matrices, colorants/flavoring, carriers, excipients, buffers, stabilizers, solubilizers, other materials well known in the art and combinations thereof.

Any pharmaceutically acceptable (i.e., sterile and non-toxic) liquid, semisolid, or solid diluents that serve as pharmaceutical vehicles, excipients, or media may be used. Exemplary diluents include, but are not limited to, polyoxyethylene sorbitan monolaurate, magnesium stearate, calcium phosphate, mineral oil, cocoa butter, and oil of theobroma, methyl- and propylhydroxybenzoate, talc, alginates, carbohydrates, especially mannitol, α-lactose, anhydrous lactose, cellulose, sucrose, dextrose, sorbitol, modified dextrans, gum acacia, and starch. Some commercially available diluents are Fast-Flo, Emdex, STA-Rx 1500, Emcompress and Avicell. Such compositions may influence the physical state, stability, rate of in vivo release, and rate of in vivo clearance of the PI3Kδ inhibitor compounds [see, e.g., Remington's Pharmaceutical Sciences, 18th Ed. pp. 1435-1712 (1990), which is incorporated herein by reference].

Pharmaceutically acceptable fillers can include, for example, lactose, microcrystalline cellulose, dicalcium phosphate, tricalcium phosphate, calcium sulfate, dextrose, mannitol, and/or sucrose.

Inorganic salts including calcium triphosphate, magnesium carbonate, and sodium chloride may also be used as fillers in the pharmaceutical compositions. Amino acids may be used such as use in a buffer formulation of the pharmaceutical compositions.

Disintegrants may be included in solid dosage formulations of the inhibitors. Materials used as disintegrants include but are not limited to starch including the commercial disintegrant based on starch, Explotab. Sodium starch glycolate, Amberlite, sodium carboxymethylcellulose, ultramylopectin, sodium alginate, gelatin, orange peel, acid carboxymethylcellulose, natural sponge and bentonite may all be used as disintegrants in the pharmaceutical compositions. Other disintegrants include insoluble cationic exchange resins. Powdered gums including powdered gums such as agar, Karaya or tragacanth may be used as disintegrants and as binders. Alginic acid and its sodium salt are also useful as disintegrants.

Binders may be used to hold the therapeutic agent together to form a hard tablet and include materials from natural products such as acacia, tragacanth, starch and gelatin. Others include methyl cellulose (MC), ethyl cellulose (EC) and carboxymethyl cellulose (CMC). Polyvinyl pyrrolidone (PVP) and hydroxypropylmethyl cellulose (HPMC) can both be used in alcoholic solutions to facilitate granulation of the therapeutic ingredient.

An antifrictional agent may be included in the formulation of the therapeutic ingredient to prevent sticking during the formulation process. Lubricants may be used as a layer between the therapeutic ingredient and the die wall, and these can include but are not limited to; stearic acid including its magnesium and calcium salts, polytetrafluoroethylene (PTFE), liquid paraffin, vegetable oils and waxes. Soluble lubricants may also be used such as sodium lauryl sulfate, magnesium lauryl sulfate, polyethylene glycol of various molecular weights, Carbowax 4000 and 6000.

Glidants that might improve the flow properties of the therapeutic ingredient during formulation and to aid rearrangement during compression might be added. Suitable glidants include starch, talc, pyrogenic silica and hydrated silicoaluminate.

To aid dissolution of the therapeutic into the aqueous environment, a surfactant might be added as a wetting agent. Natural or synthetic surfactants may be used. Surfactants may include anionic detergents such as sodium lauryl sulfate, dioctyl sodium sulfosuccinate, and dioctyl sodium sulfonate. Cationic detergents such as benzalkonium chloride and benzethonium chloride may be used. Nonionic detergents that can be used in the pharmaceutical formulations include lauromacrogol 400, polyoxyl 40 stearate, polyoxyethylene hydrogenated castor oil 10, 50 and 60, glycerol monostearate, polysorbate 40, 60, 65 and 80, sucrose fatty acid ester, methyl cellulose and carboxymethyl cellulose. These surfactants can be present in the pharmaceutical compositions of the invention either alone or as a mixture in different ratios.

Controlled release formulation may be desirable. The inhibitors of the invention can be incorporated into an inert matrix which permits release by either diffusion or leaching mechanisms, e.g., gums. Slowly degenerating matrices may also be incorporated into the pharmaceutical formulations, e.g., alginates, polysaccharides. Another form of controlled release is a method based on the Oros therapeutic system (Alza Corp.), i.e., the drug is enclosed in a semipermeable membrane which allows water to enter and push the inhibitor compound out through a single small opening due to osmotic effects. Some enteric coatings also have a delayed release effect.

Colorants and flavoring agents may also be included in the pharmaceutical compositions. For example, the inhibitors of the invention may be formulated (such as by liposome or microsphere encapsulation) and then further contained within an edible product, such as a beverage containing colorants and flavoring agents.

The therapeutic agent can also be given in a film coated tablet. Nonenteric materials for use in coating the pharmaceutical compositions include methyl cellulose, ethyl cellulose, hydroxyethyl cellulose, methylhydroxy-ethyl cellulose, hydroxypropyl cellulose, hydroxypropyl-methyl cellulose, sodium carboxy-methyl cellulose, povidone and polyethylene glycols. Enteric materials for use in coating the pharmaceutical compositions include esters of phthalic acid. A mix of materials might be used to provide the optimum film coating. Film coating manufacturing may be carried out in a pan coater, in a fluidized bed, or by compression coating.

The compositions can be administered in solid, semi-solid, liquid or gaseous form, or may be in dried powder, such as lyophilized form. The pharmaceutical compositions can be packaged in forms convenient for delivery, including, for example, capsules, sachets, cachets, gelatins, papers, tablets, capsules, suppositories, pellets, pills, troches, lozenges or other forms known in the art. The type of packaging will generally depend on the desired route of administration. Implantable sustained release formulations are also contemplated, as are transdermal formulations.

In the methods according to the invention, the inhibitor compounds may be administered by various routes. For example, pharmaceutical compositions may be for injection, or for oral, nasal, transdermal or other forms of administration, including, e.g., by intravenous, intradermal, intramuscular, intramammary, intraperitoneal, intrathecal, intraocular, retrobulbar, intrapulmonary (e.g., aerosolized drugs) or subcutaneous injection (including depot administration for long term release e.g., embedded under the splenic capsule, brain, or in the cornea); by sublingual, anal, vaginal, or by surgical implantation, e.g., embedded under the splenic capsule, brain, or in the cornea. The treatment may consist of a single dose or a plurality of doses over a period of time. In general, the methods of the invention involve administering effective amounts of an inhibitor of the invention together with pharmaceutically acceptable diluents, preservatives, solubilizers, emulsifiers, adjuvants and/or carriers, as described above.

In one aspect, the invention provides methods for oral administration of a pharmaceutical composition of the invention. Oral solid dosage forms are described generally in Remington's Pharmaceutical Sciences, supra at Chapter 89. Solid dosage forms include tablets, capsules, pills, troches or lozenges, and cachets or pellets. Also, liposomal or proteinoid encapsulation may be used to formulate the compositions (as, for example, proteinoid microspheres reported in U.S. Pat. No. 4,925,673). Liposomal encapsulation may include liposomes that are derivatized with various polymers (e.g., U.S. Pat. No. 5,013,556). In general, the formulation will include a compound of the invention and inert ingredients which protect against degradation in the stomach and which permit release of the biologically active material in the intestine.

The inhibitors can be included in the formulation as fine multiparticulates in the form of granules or pellets of particle size about 1 mm. The formulation of the material for capsule administration could also be as a powder, lightly compressed plugs or even as tablets. The capsules could be prepared by compression.

Also contemplated herein is pulmonary delivery of the PI3Kδ inhibitors in accordance with the invention. According to this aspect of the invention, the inhibitor is delivered to the lungs of a mammal while inhaling and traverses across the lung epithelial lining to the blood stream.

Contemplated for use in the practice of this invention are a wide range of mechanical devices designed for pulmonary delivery of therapeutic products, including but not limited to nebulizers, metered dose inhalers, and powder inhalers, all of which are familiar to those skilled in the art. Some specific examples of commercially available devices suitable for the practice of this invention are the Ultravent nebulizer, manufactured by Mallinckrodt, Inc., St. Louis, Mo.; the Acorn II nebulizer, manufactured by Marquest Medical Products, Englewood, Colorado; the Ventolin metered dose inhaler, manufactured by Glaxo Inc., Research Triangle Park, North Carolina; and the Spinhaler powder inhaler, manufactured by Fisons Corp., Bedford, Mass.

All such devices require the use of formulations suitable for the dispensing of the inventive compound. Typically, each formulation is specific to the type of device employed and may involve the use of an appropriate propellant material, in addition to diluents, adjuvants and/or carriers useful in therapy.

When used in pulmonary administration methods, the inhibitors of the invention are most advantageously prepared in particulate form with an average particle size of less than 10 μm (or microns), for example, 0.5 to 5 μm, for most effective delivery to the distal lung.

Formulations suitable for use with a nebulizer, either jet or ultrasonic, will typically comprise the inventive compound dissolved in water at a concentration range of about 0.1 to 100 mg of inhibitor per mL of solution, 1 to 50 mg of inhibitor per mL of solution, or 5 to 25 mg of inhibitor per mL of solution. The formulation may also include a buffer. The nebulizer formulation may also contain a surfactant, to reduce or prevent surface induced aggregation of the inhibitor caused by atomization of the solution in forming the aerosol.

Formulations for use with a metered-dose inhaler device will generally comprise a finely divided powder containing the inventive inhibitors suspended in a propellant with the aid of a surfactant. The propellant may be any conventional material employed for this purpose, such as a chlorofluorocarbon, a hydrochlorofluorocarbon, a hydrofluorocarbon, or a hydrocarbon, including trichlorofluoromethane, dichlorodifluoromethane, dichlorotetrafluoroethanol, and 1,1,1,2-tetrafluoroethane, or combinations thereof. Suitable surfactants include sorbitan trioleate and soya lecithin. Oleic acid may also be useful as a surfactant.

Formulations for dispensing from a powder inhaler device will comprise a finely divided dry powder containing the inventive compound and may also include a bulking agent or diluent such as lactose, sorbitol, sucrose, mannitol, trehalose, or xylitol in amounts which facilitate dispersal of the powder from the device, e.g., 50 to 90% by weight of the formulation.

Nasal delivery of the inventive compound is also contemplated. Nasal delivery allows the passage of the inhibitor to the blood stream directly after administering the therapeutic product to the nose, without the necessity for deposition of the product in the lung. Formulations for nasal delivery may include dextran or cyclodextran. Delivery via transport across other mucous membranes is also contemplated.

Toxicity and therapeutic efficacy of the PI3Kδ selective compounds can be determined by standard pharmaceutical procedures in cell cultures or experimental animals, e.g., for determining the LD50 (the dose lethal to 50% of the population) and the ED50 (the dose therapeutically effective in 50% of the population). Additionally, this information can be determined in cell cultures or experimental animals additionally treated with other therapies including but not limited to radiation, chemotherapeutic agents, photodynamic therapies, radiofrequency ablation, anti-angiogenic agents, and combinations thereof.

In practice of the methods of the invention, the pharmaceutical compositions are generally provided in doses ranging from 1 pg compound/kg body weight to 1000 mg/kg, 0.1 mg/kg to 100 mg/kg, 0.1 mg/kg to 50 mg/kg, and 1 to 20 mg/kg, given in daily doses or in equivalent doses at longer or shorter intervals, e.g., every other day, twice weekly, weekly, or twice or three times daily. The inhibitor compositions may be administered by an initial bolus followed by a continuous infusion to maintain therapeutic circulating levels of drug product. Those of ordinary skill in the art will readily optimize effective dosages and administration regimens as determined by good medical practice and the clinical condition of the individual to be treated. The frequency of dosing will depend on the pharmacokinetic parameters of the agents and the route of administration. The optimal pharmaceutical formulation will be determined by one skilled in the art depending upon the route of administration and desired dosage [see, for example, Remington's Pharmaceutical Sciences, pp. 1435-1712, the disclosure of which is hereby incorporated by reference]. Such formulations may influence the physical state, stability, rate of in vivo release, and rate of in vivo clearance of the administered agents. Depending on the route of administration, a suitable dose may be calculated according to body weight, body surface area or organ size. Further refinement of the calculations necessary to determine the appropriate dosage for treatment involving each of the above mentioned formulations is routinely made by those of ordinary skill in the art without undue experimentation, especially in light of the dosage information and assays disclosed herein, as well as the pharmacokinetic data observed in human clinical trials. Appropriate dosages may be ascertained by using established assays for determining blood level dosages in conjunction with an appropriate physician considering various factors which modify the action of drugs, e.g., the drug's specific activity, the severity of the indication, and the responsiveness of the individual, the age, condition, body weight, sex and diet of the individual, the time of administration and other clinical factors. As studies are conducted, further information will emerge regarding the appropriate dosage levels and duration of treatment for various indications involving aberrant proliferation of hematopoietic cells.

### EXAMPLES

The following examples are provided merely to illustrate the invention, and thus are not intended to limit the scope thereof.

### Examples 1

### Western Blot Analyses of Hematopoietic Cells

In order to investigate whether specific therapeutic targets could be identified in AML cells, the expression and activation of signaling proteins involved in the PI3K pathway was determined by Western blot within primary blast cells isolated from 64 AML patients.

Additionally, the expression of the p100α, p110β, and p110δ isoforms of the class Ia PI3Ks was studied by western blot in 7 PI3K+ cell samples (defined as cells in which PI3K/Akt pathway is constitutively active) and 1 PI3K− cell samples (defined as cells in which PI3K/Akt pathway is not constitutively active, but is activatable), using isoform-specific antibodies. Controls for the p110α, p110β, and p110δ isoforms were p110α recombinant, p110β recombinant and p110δ recombinant proteins, respectively. These Western blot analyses demonstrated that the p110δ isoform of PI3K is consistently expressed in certain AML patients.

The following procedure was used for the various Western blot analyses.

Bone marrow cells from newly diagnosed AML patients were obtained from the different centers of the Groupe Ouest-Est des Leucemies et des Autres Maladies du Sang (GOELAMS) in the setting of the LAM2001 French Multicenter protocol. All patients with AML secondary to myelodysplasia were issued from the Hematology unit of Cochin Hospital (Paris). Fifty-four patients with primary AML at diagnosis and 10 patients with AML secondary to a myelodysplastic syndrome were included in this study. The bone marrow cells were obtained with informed consent under an Institutional Review Board-approved protocol. All samples were obtained before induction of chemotherapy. Diagnosis and classification of AML were based on the criteria of the FAB sub-types. The FAB classification was MO (n=8), M1 (n=10) M2 (n=21), M4 (n=10), M4eo (n=2), M5 (n=11), unknown AML (n=2), and included 26 female and 38 men, with a median age of 52 years. M3, M6 and M7 FAB subtypes were not included in this study.

The bone marrow samples were subjected to Ficoll-Hypaque density gradient separation to isolate mononuclear cells (BMMCs). The BMMCs of the AML patients were washed three times in PBS, and diluted at 10⁷/ml in serum-free medium. To assess whether phosphorylation of signaling proteins was constitutive, the BMMCs of the AML patients (hereinafter, “AML cells”) were then cultured at 37° C. in serum free medium for four hours. The AML cells were incubated with or without different inhibitors (LY294002 at 25 μM (Sigma), rapamycin (Sigma) at 50 ng/ml, or a PI3Kδ selective inhibitor at 10 μM) for 30 minutes at 37° C. In all samples, cell viability was tested by stimulation with stem cell factor (SCF) for 15 minutes at 50 ng/ml. The experiments were terminated by diluting the cells with ice-cold PBS containing 50 μmol/L sodium orthovanadate. Cells were then directly boiled in Laemmli sample buffer, separated by SDS-PAGE, and analyzed by Western blot (WB) using the appropriate antibodies. The antibodies were detected using chemoluminescence kit ECL (Amersham Pharmacia Biotech®). The SuperSignal® West Femto Maximum Sensitivity Substrate Kit was used (PIERCE, prod. no. 34095) for antibodies directed against phosphorylated proteins.

Akt and FOXO3a Protein Western Blot Analyses

Western blot experiments were conducted to determine the activation status of the PI3K pathway in AML cells. Specifically, the expression and phosphorylation status of Akt and FOXO3a proteins, two downstream targets of PI3K, were determined using anti-pAkt (Ser473) (Cell Signaling Technology) and anti-pFOXO3a (Thr 32) (Upstate Biotechnology) antibodies, and reprobed with anti-actin antibody (Sigma, catalog number A5441).

The levels of Akt and FOXO3a remained unchanged after serum starvation in 58% of the cell samples tested, and thus the PI3K pathway was deemed to be constitutively activated (“PI3K-positive samples”) in these cells. In other cell samples, activation of the PI3K pathway was not observed, and the phosphorylation of Akt and FOXO3a proteins was only induced upon SCF stimulation (“PI3K-negative samples”). Additionally, pretreatment of cells with LY294002, a broad spectrum PI3K inhibitor, completely abolished phosphorylation of Akt and FOXO3a. No difference in percentage of PI3K activation was observed between samples of de novo (31/54) and secondary (6/10) AML cells. In addition, PI3K activation was equally observed across the different AML subgroups, classified according top the FAB guidelines (see Table 1).

These Western blot experiments demonstrate that constitutive activation of the PI3K pathway in primary blasts from the bone marrow of AML patients was detected in 58% of the AML cell samples.

TABLE 1
DISTRIBUTION OF PI3K-POSITIVITY IN THE 64
AML SAMPLES ACCORDING TO THE FAB SUBTYPE.

| FAB | P13K⁺/total samples |
|---|---|
| M0 | 3/8 |
| M1 | 3/10 |
| M2 | 14/21 |
| M4 | 6/10 |
| M4eo | 2/2 |
| M5 | 9/11 |
| Not determined | 0/2 |

PTEN and SHIP-1 Protein Western Blot Analyses

The phosphatase and tensin homologue on chromosome ten (PTEN) is a negative regulator of PI3K and a potent tumor suppressor that is inactivated in various cancers [Dahia et al., Hum. Mol. Genet., 8:185-193 (1999)]. PTEN protein negatively regulates Akt activation through phosphoinositide (3,4,5) trisphosphate (“PI(3,4,5)P3”) dephosphorylation, and loss of PTEN activity leads to constitutive activation of the PI3K/Akt pathway, as is seen in advanced stages of several human tumors [Cantley et al., Proc. Natl. Acad. Sci. (USA), 96:4240-4245 (1999); Luo et al., Cancer Cell, 4:257-262 (2003)]. Similarly, the SH2-containing inositol 5′ phosphatase (SHIP-1) is an additional tumor suppressor in myeloid leukemogenesis [Luo et al., Leukemia, 17:1-8 (2003)].

To investigate whether the aforementioned tumor suppressing phosphoinositide phosphatases are normally expressed in AML cells, Western blot experiments were conducted to determine their expression using anti-SHIP1 (Santa Cruz Biotechnology, catalog number 6244) and anti-PTEN (Santa Cruz Biotechnology, catalog number 7974) antibodies.

In all AML patients investigated (39 out of 64), PTEN protein was present. Similarly, SHIP-1 was always detected in PI3K-positive patients. While these phosphatases were present, it is conceivable that they are not active or suffer reduced activity from point mutations or inappropriate negative regulation. Taken together, these data suggest that phosphoinositide phosphatases are normally expressed in AML cells and that the mechanisms leading to PI3K constitutive activation in AML cells may involve the deregulation of an upstream receptor(s) or cytoplasmic signalling proteins.

GAB1 and GAB2 Protein Western Blot Analyses

The Grb2-associated binder family proteins (GAB) GAB1 and GAB2 are involved in PI3K activation after cytokine stimulation [Lecoq-Lafon et al., Blood, 93:2578-2585 (1999); Bouscary et al., Oncogene, 20:2197-2204 (2001); Rodrigues et al., Mol. Cell Biol., 20:1448-1459 (2000); Kong et al., J. Biol. Chem., 275:36035-36042 (2000)]. Furthermore, GAB2 and its associated proteins have been identified as key determinants of the Bcr-Abl transformation in chronic myeloid leukemia [Gu et al., Mol. Cell Biol., 20:7109-7120 (2000)].

Western blot experiments were conducted to determine the expression and phosphorylation status of GAB1 and GAB2 proteins, two downstream targets of PI3K, in AML cells. Anti-GAB1 (United Biomedical Inc., catalog number 06-579), anti-pGAB1 (Tyr 627) (Santa Cruz Biotechnology (catalog number 12961-R) antibodies were used. The anti-pGAB1 antibody also recognizes pGAB2.

Both GAB1 and GAB2 proteins were found to be phosphorylated on tyrosine in the majority of PI3K-positive AML cell samples, whereas tyrosine phosphorylation of GAB1/2 proteins was only detected after SCF stimulation in PI3K-negative cell samples. These Western blot analyses further demonstrate that the PI3K/Akt pathway is constitutively active in a subset of AML cell samples, and strongly indicates the activation of an upstream kinase such as FLT3 (see Example 6) in indications involving aberrant proliferation of hematopoietic cells.

### Examples 2

### Confocal Microscopic Analysis

Phosphorylation of Akt and FOXO3A were also determined by confocal microscopy performed on bone marrow cytospins (BMMCs centrifuged onto glass slides) deprived for 4 h in serum-free medium. The Ser473 phosphorylation status of Akt was also assessed on cytospins of cells treated as described above for immunoblot analysis.

Confocal microscopy was performed using an inverted scanning confocal microscope equipped with UV as well as visible illumination (488 nm, 568 nm, and 647 nm) (Biorad MRC 1024 coupled with a NIKON diaphot 300 inverted microscope). Frozen or fresh bone marrow samples (cytospins) were fixed with PBS containing 4% paraformaldehyde, permeabilized with PBS containing 0.1% Triton, blocked for 45 min with PBS containing 5% nonfat dry milk; and incubated in primary anti-phospho Akt antibody (Cell Signaling 9277; used at 1/100 dilution ratio in PBS containing 5% nonfat dry milk) or anti-phospho FOXO3A antibody (Cell Signaling 9464; used at 1/100 dilution ratio in PBS containing 5% nonfat dry milk). Slides were then stained with Texas Red conjugated donkey anti-rabbit secondary antibody (Jackson Immunoresearch 711-075-152; used at 1/100 dilution ratio in PBS containing 5% nonfat dry milk). Nuclei were stained using 4′,6-Diamidino-2-phenylndole (DAPI) (1/250 dilution ratio in PBS). Slides were mounted using polyvinyl alcohol mounting media with 1,4-Diazabicyclo[2.2.2]octane (DABCO) (Fluka, product no. 10981) and stored at 4° C.

Phosphorylated Akt was detected on smears of a PI3K-positive cell samples (as defined by Western blot) whereas no signal was observed in a PI3K-negative cell samples. Phosphorylation of FOXO3A was also detected by confocal microscopy in PI3K-postive cytospins. These data further demonstrate that the PI3K/Akt pathway is constitutively activated in certain AML cell samples.

### Examples 3

### Flow Cytometric Analysis

CD34 is transmembrane protein whose expression is essentially restricted to hematopoietic progenitor cells. CD34 is also known to be expressed by AML cells and ALL cells.

Flow cytometric analysis was used to determine whether phospho-Akt and CD34 proteins were expressed by AML cells using fresh or frozen bone marrow samples, as described in Example 1.

Approximately 3×105 AML cells were incubated for 15 min with anti CD34-phycoerythrin conjugated antibody (Becton Dickinson) or isotypic control, then fixed for 15 minutes using PBS contiaing 5.5% formaldehyde. The cells were then collected by centrifugation and washed with 4 ml PBS. The cells were permeabilized for 5 minutes with Intraprep reagent 2 (Immunotech), and stained for 30 minutes with primary antibodies anti-phospho-Akt (catalog number 9277, Cell Signaling) or rabbit anti-human IgG (catalog number 19764, Sigma). The cells were then washed with 4 ml PBS and incubated for 30 minutes with goat anti-rabbit FITC-conjugated secondary antibody (catalog number F1262, Sigma). The stained cells were washed with 4 ml PBS, resuspended in 0.5 ml PBS, and analyzed using an EPICS-XL flow cytometer (Beckman Coulter).

This flow cytometric analysis demonstrated that PI3K activation occurred in the blast cell population, as CD34+ cells were also positive for Akt phosphorylation in patients with less than 70% blast cells. These data further demonstrate that the PI3K/Akt pathway is constitutively active in certain AML cell samples

### Examples 4

### Cell Proliferation Assays

Multiple studies support a role for the PI3K/Akt pathway in both proliferation and cell survival. The mammalian target of rapamycin (mTOR) serine/threonine kinase is downstream of PI3K/Akt [Manning et al., Mol. Cell, 10:151-162 (2002)] and phosphorylates P70S6 kinase (P70S6K) and eukaryotic initiation factor 4E-binding protein-1 (4E-BP1), both of which regulate mRNA translation [Gingras et al., Genes Dev., 15:807-826 (2001); Gingras et al., Genes Dev., 15:2852-2864 (2001)].

mTOR activation by Akt constitutes a process showing a linkage between both signaling pathways [Li et al., Trends Biochem. Sci., 29:32-38 (2004)]. Some of the transforming effects of PI3K/Akt promoting cell cycle and growth are mediated by the mTOR/P70S6K pathway [Podsypanina et al., Proc. Natl. Acad. Sci. (USA), 98:10320-10325 (2001); Neshat et al., Proc. Natl. Acad. Sci. (USA), 98:10314-10319 (2001); Aoki et al., Proc. Natl. Acad. Sci. (USA), 98:136-141 (2001)].

Cell proliferation assays were conducted to determine whether PI3K inhibition contributes to AML cell viability. In most PI3K-positive samples tested, the mTOR pathway was activated as assessed by phosphorylation of P70S6K protein on Threonine 389. Thus, proliferation assays were also conducted to determine whether mTOR contributes to AML cell viability. Accordingly, proliferation assays were performed on AML cells with or without the following inhibitor compounds: LY294002, a PI3Kδ selective inhibitor, rapamycin, or a combination of the PI3Kδ selective inhibitor and rapamycin. Cell proliferation was determined by measuring DNA synthesis by [3H]-Thymidine incorporation. Comparative experiments were performed on CD34+ cord blood cells.

Normal human CD34+ cells were obtained with informed consent under an Institutional Review Board-approved protocol, and were purified according to the methods described by Freyssinier et al [Freyssinier et al., Br. J. Haematol., 106:912-922 (1999)]. Briefly, umbilical cord blood units (mean volume 85 ml) from normal full-term deliveries were obtained, after informed consent of the mothers. Cord blood units were diluted with 50 ml phosphate buffer saline containing 1% bovine serum albumin (BSA) (StemCell Technologies, Vancouver, Canada) and submitted to Ficoll density gradient centrifugation. Low-density cells were recovered and CD34+ cells were separated by two cycles of positive selection using an immunomagnetic procedure (MACS, CD34 isolation kit, Miltenyi Biotech). Purification (>90%) was assessed by flow cytometric analysis with an anti-CD34 antibody (Becton Dickinson).

For AML cells (obtained as previously described in Example 1), BMMCs were cultured at 3×10⁵/ml in alpha medium containing 5% fetal calf serum (FCS) without cytokines for 48 hours, with or without the following inhibitors: LY294002 at 25 μM, PI3Kδ selective inhibitor at 10 μM, rapamycin at 50 ng/ml, or an association of a PI3Kδ selective inhibitor and rapamycin, and incubated in 96-well plates in triplicate. [³H]-Thymidine was added for a final 6 hour pulse, and the amount of radioactivity incorporated in cells was determined by trichloracetic acid (TCA) precipitation. For CD34+ cord blood cells, 5×10⁵/ml CD34+ cells were cultured in SCF (20 ng/ml), FLT3-L (10 ng/ml) and thrombopoietin (20 nM) for 48 hours with or without LY294002 at 25 μM or IC87114 at 10 μM. DNA synthesis was measured by [³H]-Thymidine incorporation after 12 h.

The concentration of PI3Kδ selective inhibitor necessary to to inhibit Akt phosphorylation was determined. The PI3Kδ selective inhibitor induced dose-dependent inhibition of Akt phosphorylation when used at increasing concentrations from 0.1 μM to 10 μM in a representative cell sample (AML5; 85% blasts). Maximum inhibition was observed at 10 μM and was sustained down to 1 μM. Based on these preliminary results, PI3Kδ selective inhibitor was administered at 10 μM to 7 PI3K-positive cell samples. The PI3Kδ selective inhibitor suppressed Akt and FOXO3a phosphorylation to the same extent as LY294002, and inhibited the proliferation of leukemic cells for representative cell samples by about 70%.

In contrast, proliferation of blast cells of representative PI3K-negative cell samples was not significantly affected. In one instance, the proliferation of leukemic cells from a representative PI3K-negative cell sample that expressed all three class IA PI3K isoforms was not significantly inhibited by administration of the PI3Kδ selective inhibitor (mean values of 25% inhibition).

Rapamycin inhibited cell growth in PI3K-positive patients (mean values of 64% inhibition) whereas its effect was moderate in a representative PI3K-negative cell sample (mean values of 29%). Administration of a combination comprising a PI3Kδ selective inhibitor and rapamycin significantly reduced proliferation over treatment with each agent alone. Furthermore, in some AML cell samples a synergistic or greater than additive anti-proliferative effect was obtained using the combination of a PI3Kδ selective inhibitor and rapamycin, as determined by multiplying the reduction in cell proliferation achieved by each modality treatment individually to yield an expected value if the effects of each treatment modality were additive.

In contrast, proliferation of normal CD34+ progenitors isolated from cord blood was not substantially effected by administration of PI3Kδ selective inhibitor, whereas administration of LY294002 completely blocked CD34+ progenitor proliferation.

In addition, PI3Kδ selective inhibitor at 10 μM did not inhibit myeloid and erythroid colony formation in methylcellulose clonogenic assays whereas LY294002 caused a 95% inhibition in this assay.

These data demonstrate that the presence of constitutive PI3K activation provides a growth advantage to AML cells when compared to PI3K negative samples, and therefore indicate that p110δ activity is required for tumor cell expansion. Additionally, the specificity of action of the PI3Kδ selective inhibitor (as demonstrated by its minimal anti-proliferative effect on normal hematopoietic progenitors relative to the profound inhibition induced by LY294002) suggests that a high therapeutic index (i.e., the dose ratio between toxic and therapeutic effects that is expressed as the ratio of the dose resulting in dose-limiting toxicity and the therapeutically effective dose could be obtained.

### Examples 5

### Apoptosis Assays

Based on data generated using LY294002, it has been suggested that PI3K controlled survival of myeloid leukemias [Xu et al., supra; Zhao et al., Leukemia, 18:267-275 (2004)]. To determine whether p110δ inhibition contributes to cell survival in addition to proliferation, apoptosis assays were conducted in AML cells.

BMMCs from two AML patients were cultured at 2×10⁵/ml in alpha medium with 5% FCS without cytokines for 24 h with or without LY294002 at 25 μM, or IC87114 at 1 μM, or rapamycin at 50 ng/ml or both IC87114 and rapamycin. The number of AML cells undergoing apoptosis was quantified by FACS analysis as the percentage of Annexin-V-PE positive cells in the whole population at 24 hours. If Annexin-V binds to the cell, cell death by apoptotic mechanisms is imminent.

Despite completely inhibiting Akt and FOXO3a phosphorylation, administration of the PI3Kδ selective inhibitor did not induce apoptosis in PI3K+ leukemic blast cells in contrast to LY294002, which induced apoptosis in the cells. Similarly, rapamycin alone or in combination with the PI3Kδ selective inhibitor did not induce apoptosis.

These data demonstrate that the inhibitory effect of the PI3Kδ selective inhibitor did not correlate with an effect on cell survival. Despite completely inhibiting Akt and FOXO3a phosphorylation, the PI3Kδ selective inhibitor did not induce cell death in AML cells. Thus, leukemic cell death is not controlled by the p110δ isoform of PI3K. The apoptosis induced by LY294002 may rely on its ability to inhibit all class I PI3K isoforms and/or its effects on PI3K-related kinases such as DNA-PK and ATM/Atr.

Therefore, taken together with the data of Example 4, these data indicate that p110δ activity is required for AML cell expansion, but seems to be dispensable for AML cell survival.

### Examples 6

### Analysis of FLT3 Mutations

The GAB adapter proteins, which function in several tyrosine signalling pathways, were also constitutively phosphorylated in the majority of the PI3K-positive samples tested but not in the PI3K-negative samples (see Example 1). These data strongly indicate that a kinase upstream of PI3K is responsible for constitutive activation of the PI3K pathway. Deregulation of the PI3K signaling pathway could be due to a mutation and constitutive activation of the class III receptor tyrosine kinase FLT3-internal tandem duplication (FLT3-ITD), reported to be present in approximately 30% of cases of AML [Gilliland et al., Blood 100: 1532-1542 (2002)]. Further, FLT3-ITD has been found to be the most common genetic lesion in AML, and can cause constitutive tyrosine kinase activity. Accordingly, AML cell samples were screened to determine whether FLT3-ITD mutations were responsible for the deregulation of the PI3K pathway (i.e., constitutive PI3K activation).

Genomic DNA was prepared using a desalting procedure as previously described [Miller et al., Nucleic Acids Res., 16:1215 (1988)]. Genomic amplification of exons 14 and 15 (formerly designed as exons 11 and 12) of the FLT3 gene was performed using the primers 11F and 12 R (or 11F and 11 R in order to control positive FLT3-ITD mutated patients), previously described [Nakao et al., Leukemia, 10:1911-1918 (1996)]. The primer 11F was 5′ end-labeled by a 6 FAM fluorescent marker. Gene Scan software (Applied Biosystems) was used to detect the wild-type allele at the expected 340 bp and an ITD allele as a larger amplified fragment. The ratio between mutated (FLT3-ITD) and wild type (WT-FLT3) alleles was also determined.

For an analysis of mutation of the exon 20 TDK domain of FLT3, all samples were screened for the codon 835 mutation or 836 deletion using the primers 20A and E20IR previously described [Abu-Duhier et al., Br. J. Haematol., 113:983-988 (2001)]. The wild type products were digested to 2 fragments of 129 and 65 bp while those containing a mutation D835/I836 yielded an undigested band of 194 bp.

Seven ITD (Internal Tandem Duplication) mutations were identified out of the 30 samples analyzed (23%). These results are within the range reported in recent studies [Stirewalt et al., Nat. Rev. Cancer, 3:650-665 (2003)]. No D835/I836 point mutation was found in the 30 cell samples analyzed. Additionally, among the 7 FLT3-ITD positive samples, only one was found associated with constitutive PI3K activation. Therefore, these data suggest that FLT3 receptor mutations are not responsible for constitutive PI3K activation.

What is claimed:

1. A method for treating and/or preventing aberrant proliferation of hematopoietic cells, comprising:
selectively inhibiting phosphoinositide 3-kinase delta (PI3Kδ) activity in hematopoietic cells, thereby treating and/or preventing aberrant proliferation of hematopoietic cells.

2. The method of claim 1, wherein inhibiting comprises administering an amount of a PI3Kδ selective inhibitor effective to inhibit PI3Kδ activity of hematopoietic cells.

3. The method of claim 1, wherein said inhibiting is in vitro.

4. The method of claim 1, wherein said inhibiting is performed in an individual in need thereof.

5. The method of claim 4, wherein the PI3K pathway is constitutively activated in the hematopoietic cells.

6. The method of claim 5, wherein the individual has an indication involving aberrant proliferation of lymphoid and/or myeloid progenitor cells.

7. The method of claim 6, wherein the indication is selected from the group consisting of acute lymphoblastic leukemia; acute myeloid leukemia; chronic lymphocytic leukemia; chronic myelogenous leukemia; myeloproliferative syndromes; myelodysplastic syndromes; cutaneous T-Cell lymphoma; hairy cell leukemia; Hodgkin's lymphoma; non-Hodgkin's lymphoma; non-Hodgkin's Lymphoma, B-Cell; non-Hodgkin's lymphoma, T-Cell; and, plasma cell neoplasms.

8. The method of claim 7, wherein the indication is selected from the group consisting of acute lymphoblastic leukemia; acute myeloid leukemia; chronic lymphocytic leukemia; chronic myelogenous leukemia; and, hairy cell leukemia.

9. The method of claim 1, further comprising administering a mammalian target of rapamycin (mTOR) inhibitor.

10. The method of claim 9, wherein the mTOR inhibitor is selected from the group consisting of rapamycin, FK506, cyclosporine A (CsA), and everolimus.

11. The method of claim 2, wherein the PI3Kδ selective inhibitor is administered in an amount effective to inhibit Akt phosphorylation.

12. The method of claim 2, wherein the PI3Kδ selective inhibitor is administered in an amount effective to inhibit FOXO3a phosphorylation.

13. The method of claim 2, wherein the PI3Kδ selective inhibitor is administered in an amount effective to inhibit GAB1 phosphorylation.

14. The method of claim 2, wherein the PI3Kδ selective inhibitor is administered in an amount effective to inhibit GAB2 phosphorylation.

15. The method of claim 2, wherein the PI3Kδ selective inhibitor is a compound having formula (I) or pharmaceutically acceptable salts and solvates thereof: 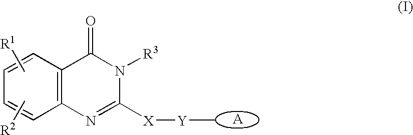
wherein A is an optionally substituted monocyclic or bicyclic ring system containing at least two nitrogen atoms, and at least one ring of the system is aromatic;
X is selected from the group consisting of C(R^{b})₂, CH₂CHR^{b}, and CH═C(R^{b});
Y is selected from the group consisting of null, S, SO, SO₂, NH, O, C(═O), OC(═O), C(═O)O, and NHC(═O)CH₂S;
R¹ and R², independently, are selected from the group consisting of hydrogen, C₁₋₆alkyl, aryl, heteroaryl, halo, NHC(═O)C₁₋₃alkyleneN(R^{a})₂, NO₂, OR^{a}, CF₃, OCF₃, N(R^{a})₂, CN, OC(═O)R^{a}, C(═O)R^{a}, C(═O)OR^{a}, arylOR^{b}, Het, NR^{a}C(═O)C₁₋₃alkyleneC(═O)OR^{a}, arylOC₁₋₃alkyleneN(R^{a})₂, arylOC(═O)R^{a}, C₁₋₄alkyleneC(═O)OR^{a}, OC₁₋₄alkyleneC(═O)OR^{a}, C₁₋₄alkyleneOC₁₋₄alkyleneC(═O)OR^{a}, C(═O)NR^{a}SO₂R^{a}, C₁₋₄alkyleneN(R^{a})₂, C₂₋₆alkenyleneN(R^{a})₂, C(═O)NR^{a}C₁₋₄alkyleneOR^{a}, C(═O)NR^{a}C₁₋₄alkyleneHet, OC₂₋₄alkyleneN(R^{a})₂, OC₁₋₄alkyleneCH(OR^{b})CH₂N(R^{a})₂, OC₁₋₄alkyleneHet, OC₂₋₄alkyleneOR^{a}, OC₂₋₄alkyleneNR^{a}C(═O)OR^{a}, NR^{a}C₁₋₄alkyleneN(R^{a})₂, NR^{a}C(═O)R^{a}, NR^{a}C(═O)N(R^{a})₂, N(SO₂C₁₋₄alkyl)₂, NR^{a}(SO₂C₁₋₄alkyl), SO₂N(R^{a})₂, OSO₂CF₃, C₁₋₃alkylenearyl, C₁₋₄alkyleneHet, C₆alkyleneOR^{b}, C₁₋₃alkyleneN(R^{a})₂, C(═O)N(R^{a})₂, NHC(═O)C₁₋₃alkylenearyl, C₃₋₈cycloalkyl, C₃₋₈heterocycloalkyl, arylOC₁₋₃alkyleneN(R^{a})₂, arylOC(═O)R b, NHC(═O)C₁₋₃alkyleneC₃₋₈heterocycloalkyl, NHC(═O)C₁₋₃alkyleneHet, OC₁₋₄alkyleneOC₁₋₄alkyleneC(═O)OR^{b}, C(═O)C₁₋₄alkyleneHet, and NHC(═O)haloC₁₋₆alkyl;
or R¹ and R² are taken together to form a 3- or 4-membered alkylene or alkenylene chain component of a 5- or 6-membered ring, optionally containing at least one heteroatom;
R³ is selected from the group consisting of optionally substituted hydrogen, C₁₋₆alkyl, C₃₋₈cycloalkyl, C₃₋₈heterocycloalkyl, C₁₋₄alkylenecycloalkyl, C₂₋₆alkenyl, C₁₋₃alkylenearyl, arylC₁₋₃alkyl, C(═O)R^{a}, aryl, heteroaryl, C(═O)OR^{a}, C(═O)N(R^{a})₂, C(═S)N(R^{a})₂, SO₂R^{a}, SO₂N(R^{a})₂, S(═O)R^{a}, S(═O)N(R^{a})₂, C(═O)NR^{a}C₁₋₄alkyleneOR^{a}, C(═O)NR^{a}C₁₋₄alkyleneHet, C(═O)C₁₋₄alkylenearyl, C(═O)C₁₋₄alkyleneheteroaryl, C₁₋₄alkylenearyl optionally substituted with one or more of halo, SO₂N(R^{a})₂, N(R^{a})₂, C(═O)OR^{a}, NR^{a}SO₂CF₃, CN, NO₂, C(═O)R^{a}, OR^{a}, C₁₋₄alkyleneN(R^{a})₂, and OC₁₋₄alkyleneN(R^{a})₂, C₁₋₄alkyleneheteroaryl, C₁₋₄alkyleneHet, C₁₋₄alkyleneC(═O)C₁₋₄alkylenearyl, C₁₋₄alkyleneC(═O)C₁₋₄alkyleneheteroaryl, C₁₋₄alkyleneC(═O)Het, C₁₋₄alkyleneC(═O)N(R^{a})₂, C₁₋₄alkyleneOR^{a}, C₁₋₄alkyleneNR^{a}C(═O)R^{a}, C₁₋₄alkyleneOC₁₋₄alkyleneOR^{a}, C₁₋₄alkyleneN(R^{a})₂, C₁₋₄alkyleneC(═O)OR^{a}, and C₁₋₄alkyleneOC₁₋₄alkyleneC(═O)OR^{a};
R^{a} is selected from the group consisting of hydrogen, C₁₋₆alkyl, C₃₋₈cycloalkyl, C₃₋₈heterocycloalkyl, C₁₋₃alkyleneN(R^{c})₂, aryl, arylC₁₋₃alkyl, C₁₋₃alkylenearyl, heteroaryl, heteroarylC₁₋₃alkyl, and C₁₋₃alkyleneheteroaryl;
or two R^{a} groups are taken together to form a 5- or 6-membered ring, optionally containing at least one heteroatom;
R^{b} is selected from the group consisting of hydrogen, C₁₋₆alkyl, heteroC₁₋₃alkyl, C₁₋₃alkyleneheteroC₁₋₃alkyl, arylheteroC₁₋₃alkyl, aryl, heteroaryl, arylC₁₋₃alkyl, heteroarylC₁₋₃alkyl, C₁₋₃alkylenearyl, and C₁₋₃alkyleneheteroaryl;
R^{c} is selected from the group consisting of hydrogen, C₁₋₆alkyl, C₃₋₈cycloalkyl, aryl, and heteroaryl; and,
Het is a 5- or 6-membered heterocyclic ring, saturated or partially or fully unsaturated, containing at least one heteroatom selected from the group consisting of oxygen, nitrogen, and sulfur, and optionally substituted with C₁₋₄alkyl or C(═O)OR^{a}.

16. The method of claim 2, wherein the PI3Kδ selective inhibitor is selected from the group consisting of:
2-(6-aminopurin-9-ylmethyl)-3-(2-chlorophenyl)-6,7-dimethoxy-3H-quinazolin-4-one; 2-(6-aminopurin-o-ylmethyl)-6-bromo-3-(2-chlorophenyl)-3H-quinazolin-4-one; 2-(6-aminopurin-o-ylmethyl)-3-(2-chlorophenyl)-7-fluoro-3H-quinazolin-4-one; 2-(6-aminopurin-9-ylmethyl)-6-chloro-3-(2-chlorophenyl)-3H-quinazolin-4-one; 2-(6-aminopurin-9-ylmethyl)-3-(2-chlorophenyl)-5-fluoro-3H-quinazolin-4-one; 2-(6-aminopurin-o-ylmethyl)-5-chloro-3-(2-chloro-phenyl)-3H-quinazolin-4-one; 2-(6-aminopurin-9-ylmethyl)-3-(2-chlorophenyl)-5-methyl-3H-quinazolin-4-one; 2-(6-aminopurin-9-ylmethyl)-8-chloro-3-(2-chlorophenyl)-3H-quinazolin-4-one; 2-(6-aminopurin-9-ylmethyl)-3-biphenyl-2-yl-5-chloro-3H-quinazolin-4-one; 5-chloro-2-(9H-purin-6-ylsulfanylmethyl)-3-o-tolyl-3H-quinazolin-4-one; 5-chloro-3-(2-fluorophenyl)-2-(9H-purin-6-yl-sulfanylmethyl)-3H-quinazolin-4-one; 2-(6-aminopurin-9-ylmethyl)-5-chloro-3-(2-fluorophenyl)-3H-quinazolin-4-one; 3-biphenyl-2-yl-5-chloro-2-(9H-purin-6-ylsulfanylmethyl)-3H-quinazolin-4-one; 5-chloro-3-(2-methoxyphenyl)-2-(9H-purin-6-yl-sulfanylmethyl)-3H-quinazolin-4-one; 3-(2-chlorophenyl)-5-fluoro-2-(9H-purin-6-yl-sulfanylmethyl)-3H-quinazolin-4-one; 3-(2-chlorophenyl)-6,7-dimethoxy-2-(9H-purin-6-yl-sulfanylmethyl)-3H-quinazolin-4-one; 6-bromo-3-(2-chlorophenyl)-2-(9H-purin-6-yl-sulfanylmethyl)-3H-quinazolin-4-one; 3-(2-chlorophenyl)-8-trifluoromethyl-2-(9H-purin-6-ylsulfanylmethyl)-3H-quinazolin-4-one; 3-(2-chlorophenyl)-2-(9H-purin-6-ylsulfanylmethyl)-3H-benzo[g]quinazolin-4-one; 6-chloro-3-(2-chlorophenyl)-2-(9H-purin-6-yl-sulfanylmethyl)-3H-quinazolin-4-one; 8-chloro-3-(2-chlorophenyl)-2-(9H-purin-6-yl-sulfanylmethyl)-3H-quinazolin-4-one; 3-(2-chlorophenyl)-7-fluoro-2-(9H-purin-6-yl-sulfanylmethyl)-3H-quinazolin-4-one; 3-(2-chlorophenyl)-7-nitro-2-(9H-purin-6-yl-sulfanylmethyl)-3H-quinazolin-4-one; 3-(2-chlorophenyl)-6-hydroxy-2-(9H-purin-6-yl-sulfanylmethyl)-3H-quinazolin-4-one; 5-chloro-3-(2-chlorophenyl)-2-(9H-purin-6-yl-sulfanylmethyl)-3H-quinazolin-4-one; 3-(2-chlorophenyl)-5-methyl-2-(9H-purin-6-yl-sulfanylmethyl)-3H-quinazolin-4-one; 3-(2-chlorophenyl)-6,7-difluoro-2-(9H-purin-6-yl-sulfanylmethyl)-3H-quinazolin-4-one; 3-(2-chlorophenyl)-6-fluoro-2-(9H-purin-6-yl-sulfanylmethyl)-3H-quinazolin-4-one; 2-(6-aminopurin-9-ylmethyl)-3-(2-isopropylphenyl)-5-methyl-3H-quinazolin-4-one; 2-(6-aminopurin-9-ylmethyl)-5-methyl-3-o-tolyl-3H-quinazolin-4-one; 3-(2-fluorophenyl)-5-methyl-2-(9H-purin-6-yl-sulfanylmethyl)-3H-quinazolin-4-one; 2-(6-aminopurin-9-ylmethyl)-5-chloro-3-o-tolyl-3H-quinazolin-4-one; 2-(6-aminopurin-9-ylmethyl)-5-chloro-3-(2-methoxy-phenyl)-3H-quinazolin-4-one; 2-(2-amino-9H-purin-6-ylsulfanylmethyl)-3-cyclopropyl-5-methyl-3H-quinazolin-4-one; 3-cyclopropylmethyl-5-methyl-2-(9H-purin-6-ylsulfanylmethyl)-3H-quinazolin-4-one; 2-(6-aminopurin-9-ylmethyl)-3-cyclopropylmethyl-5-methyl-3H-quinazolin-4-one; 2-(2-amino-9H-purin-6-ylsulfanylmethyl)-3-cyclopropylmethyl-5-methyl-3H-quinazolin-4-one; 5-methyl-3-phenethyl-2-(9H-purin-6-ylsulfanylmethyl)-3H-quinazolin-4-one; 2-(2-amino-9H-purin-6-ylsulfanylmethyl)-5-methyl-3-phenethyl-3H-quinazolin-4-one; 3-cyclopentyl-5-methyl-2-(9H-purin-6-ylsulfanylmethyl)-3H-quinazolin-4-one; 2-(6-aminopurin-9-ylmethyl)-3-cyclopentyl-5-methyl-3H-quinazolin-4-one; 3-(2-chloropyridin-3-yl)-5-methyl-2-(9H-purin-6-ylsulfanylmethyl)-3H-quinazolin-4-one; 2-(6-aminopurin-9-ylmethyl)-3-(2-chloropyridin-3-yl)-5-methyl-3H-quinazolin-4-one; 3-methyl-4-[5-methyl-4-oxo-2-(9H-purin-6-ylsulfanylmethyl)-4H-quinazolin-3-yl]-benzoic acid; 3-cyclopropyl-5-methyl-2-(9H-purin-6-ylsulfanylmethyl)-3H-quinazolin-4-one; 2-(6-aminopurin-9-ylmethyl)-3-cyclopropyl-5-methyl-3H-quinazolin-4-one; 5-methyl-3-(4-nitrobenzyl)-2-(9H-purin-6-ylsulfanylmethyl)-3H-quinazolin-4-one; 3-cyclohexyl-5-methyl-2-(9H-purin-6-ylsulfanylmethyl)-3H-quinazolin-4-one; 2-(6-aminopurin-9-ylmethyl)-3-cyclohexyl-5-methyl-3H-quinazolin-4-one; 2-(2-amino-9H-purin-6-ylsulfanylmethyl)-3-cyclo-hexyl-5-methyl-3H-quinazolin-4-one; 5-methyl-3-(E-2-phenylcyclopropyl)-2-(9H-purin-6-ylsulfanylmethyl)-3H-quinazolin-4-one; 3-(2-chlorophenyl)-5-fluoro-2-[(9H-purin-6-ylamino)methyl]-3H-quinazolin-4-one; 2-[(2-amino-9H-purin-6-ylamino)methyl]-3-(2-chlorophenyl)-5-fluoro-3H-quinazolin-4-one; 5-methyl-2-[(9H-purin-6-ylamino)methyl]-3-o-tolyl-3H-quinazolin-4-one; 2-[(2-amino-9H-purin-6-ylamino)methyl]-5-methyl-3-o-tolyl-3H-quinazolin-4-one; 2-[(2-fluoro-9H-purin-6-ylamino)methyl]-5-methyl-3-o-tolyl-3H-quinazolin-4-one; (2-chlorophenyl)-dimethylamino-(9H-purin-6-ylsulfanylmethyl)-3H-quinazolin-4-one; 5-(2-benzyloxyethoxy)-3-(2-chlorophenyl)-2-(9H-purin-6-ylsulfanylmethyl)-3H-quinazolin-4-one; 6-aminopurine-9-carboxylic acid 3-(2-chlorophenyl)-5-fluoro-4-oxo-3,4-dihydro-quinazolin-2-ylmethyl ester; N-[3-(2-chlorophenyl)-5-fluoro-4-oxo-3,4-dihydro-quinazolin-2-ylmethyl]-2-(9H-purin-6-ylsulfanyl)-acetamide; 2-[1-(2-fluoro-9H-purin-6-ylamino)ethyl]-5-methyl-3-o-tolyl-3H-quinazolin-4-one; 5-methyl-2-[1-(9H-purin-6-ylamino)ethyl]-3-o-tolyl-3H-quinazolin-4-one; 2-(6-dimethylaminopurin-9-ylmethyl)-5-methyl-3-o-tolyl-3H-quinazolin-4-one; 5-methyl-2-(2-methyl-6-oxo-1,6-dihydro-purin-7-ylmethyl)-3-o-tolyl-3H-quinazolin-4-one; 5-methyl-2-(2-methyl-6-oxo-1,6-dihydro-purin-9-ylmethyl)-3-o-tolyl-3H-quinazolin-4-one; 2-(amino-dimethylaminopurin-9-ylmethyl)-5-methyl-3-o-tolyl-3H-quinazolin-4-one; 2-(2-amino-9H-purin-6-ylsulfanylmethyl)-5-methyl-3-o-tolyl-3H-quinazolin-4-one; 2-(4-amino-1,3,5-triazin-2-ylsulfanylmethyl)-5-methyl-3-o-tolyl-3H-quinazolin-4-one; 5-methyl-2-(7-methyl-7H-purin-6-ylsulfanylmethyl)-3-o-tolyl-3H-quinazolin-4-one; 5-methyl-2-(2-oxo-1,2-dihydro-pyrimidin-4-ylsulfanylmethyl)-3-o-tolyl-3H-quinazolin-4-one; 5-methyl-2-purin-7-ylmethyl-3-o-tolyl-3H-quinazolin-4-one; 5-methyl-2-purin-9-ylmethyl-3-o-tolyl-3H-quinazolin-4-one; 5-methyl-2-(9-methyl-9H-purin-6-ylsulfanylmethyl)-3-o-tolyl-3H-quinazolin-4-one; 2-(2,6-diamino-pyrimidin-4-ylsulfanylmethyl)-5-methyl-3-o-tolyl-3H-quinazolin-4-one; 5-methyl-2-(5-methyl-[1,2,4]triazolo[1,5-a]pyrimidin-7-ylsulfanylmethyl)-3-o-tolyl-3H-quinazolin-4-one; 5-methyl-2-(2-methylsulfanyl-9H-purin-6-ylsulfanylmethyl)-3-o-tolyl-3H-quinazolin-4-one; 2-(2-hydroxy-9H-purin-6-ylsulfanylmethyl)-5-methyl-3-o-tolyl-3H-quinazolin-4-one; 5-methyl-2-(1-methyl-1H-imidazol-2-ylsulfanylmethyl)-3-o-tolyl-3H-quinazolin-4-one; 5-methyl-3-o-tolyl-2-(1H-[1,2,4]triazol-3-ylsulfanylmethyl)-3H-quinazolin-4-one; 2-(2-amino-6-chloro-purin-9-ylmethyl)-5-methyl-3-o-tolyl-3H-quinazolin-4-one; 2-(6-aminopurin-7-ylmethyl)-5-methyl-3-o-tolyl-3H-quinazolin-4-one; 2-(7-amino-1,2,3-triazolo[4,5-d]pyrimidin-3-yl-methyl)-5-methyl-3-o-tolyl-3H-quinazolin-4-one; 2-(7-amino-1,2,3-triazolo[4,5-d]pyrimidin-1-yl-methyl)-5-methyl-3-o-tolyl-3H-quinazolin-4-one; 2-(6-amino-9H-purin-2-ylsulfanylmethyl)-5-methyl-3-o-tolyl-3H-quinazolin-4-one; 2-(2-amino-6-ethylamino-pyrimidin-4-ylsulfanylmethyl)-5-methyl-3-o-tolyl-3H-quinazolin-4-one; 2-(3-amino-5-methylsulfanyl-1,2,4-triazol-1-yl-methyl)-5-methyl-3-o-tolyl-3H-quinazolin-4-one; 2-(5-amino-3-methylsulfanyl-1,2,4-triazol-1-ylmethyl)-5-methyl-3-o-tolyl-3H-quinazolin-4-one; 5-methyl-2-(6-methylaminopurin-9-ylmethyl)-3-o-tolyl-3H-quinazolin-4-one; 2-(6-benzylaminopurin-9-ylmethyl)-5-methyl-3-o-tolyl-3H-quinazolin-4-one; 2-(2,6-diaminopurin-9-ylmethyl)-5-methyl-3-o-tolyl-3H-quinazolin-4-one; 5-methyl-2-(9H-purin-6-ylsulfanylmethyl)-3-o-tolyl-3H-quinazolin-4-one; 3-isobutyl-5-methyl-2-(9H-purin-6-ylsulfanylmethyl)-3H-quinazolin-4-one; N{2-[5-Methyl-4-oxo-2-(9H-purin-6-ylsulfanylmethyl)-4H-quinazolin-3-yl]-phenyl}-acetamide; 5-methyl-3-(E-2-methyl-cyclohexyl)-2-(9H-purin-6-ylsulfanylmethyl)-3H-quinazolin-4-one; 2-[5-methyl-4-oxo-2-(9H-purin-6-ylsulfanylmethyl)-4H-quinazolin-3-yl]-benzoic acid; 3-{2-[(2-dimethylaminoethyl)methylamino]phenyl}-5-methyl-2-(9H-purin-6-ylsulfanylmethyl)-3H-quin-azolin-4-one; 3-(2-chlorophenyl)-5-methoxy-2-(9H-purin-6-ylsulfanylmethyl)-3H-quinazolin-4-one; 3-(2-chlorophenyl)-5-(2-morpholin-4-yl-ethylamino)-2-(9H-purin-6-ylsulfanylmethyl)-3H-quinazolin-4-one; 3-benzyl-5-methoxy-2-(9H-purin-6-ylsulfanylmethyl)-3H-quinazolin-4-one; 2-(6-aminopurin-9-ylmethyl)-3-(2-benzyloxyphenyl)-5-methyl-3H-quinazolin-4-one; 2-(6-aminopurin-9-ylmethyl)-3-(2-hydroxyphenyl)-5-methyl-3H-quinazolin-4-one; 2-(1-(2-amino-9H-purin-6-ylamino)ethyl)-5-methyl-3-o-tolyl-3H-quinazolin-4-one; 5-methyl-2-[1-(9H-purin-6-ylamino)propyl]-3-o-tolyl-3H-quinazolin-4-one; 2-(1-(2-fluoro-9H-purin-6-ylamino)propyl)-5-methyl-3-o-tolyl-3H-quinazolin-4-one; 2-(1-(2-amino-9H-purin-6-ylamino)propyl)-5-methyl-3-o-tolyl-3H-quinazolin-4-one; 2-(2-benzyloxy-1-(9H-purin-6-ylamino)ethyl)-5-methyl-3-o-tolyl-3H-quinazolin-4-one; 2-(6-aminopurin-9-ylmethyl)-5-methyl-3-{2-(2-(1-methylpyrrolidin-2-yl)-ethoxy)-phenyl}-3H-quinazolin-4-one; 2-(6-aminopurin-9-ylmethyl)-3-(2-(3-dimethylamino-propoxy)-phenyl)-5-methyl-3H-quinazolin-4-one; 2-(6-aminopurin-9-ylmethyl)-5-methyl-3-(2-prop-2-ynyloxyphenyl)-3H-quinazolin-4-one; 2-{2-(1-(6-aminopurin-9-ylmethyl)-5-methyl-4-oxo-4H-quinazolin-3-yl]-phenoxy}-acetamide; 2-[(6-aminopurin-9-yl)methyl]-5-methyl-3-o-tolyl-3-hydroquinazolin-4-one; 3-(3,5-difluorophenyl)-5-methyl-2-[(purin-6-ylamino)methyl]-3-hydroquinazolin-4-one; 3-(2,6-dichlorophenyl)-5-methyl-2-[(purin-6-ylamino)methyl]-3-hydroquinazolin-4-one; 3-(2-Fluoro-phenyl)-2-[1-(2-fluoro-9H-purin-6-ylamino)-ethyl]-5-methyl-3-hydroquinazolin-4-one; 2-[1-(6-aminopurin-9-yl)ethyl]-3-(3,5-difluorophenyl)-5-methyl-3-hydroquinazolin-4-one; 2-[1-(7-Amino-[1,2,3]triazolo[4,5-d]pyrimidin-3-yl)-ethyl]-3-(3,5-difluoro-phenyl)-5-methyl-3H-quinazolin-4-one; 5-chloro-3-(3,5-difluoro-phenyl)-2-[1-(9H-purin-6-ylamino)-propyl]-3H-quinazolin-4-one; 3-phenyl-2-[1-(9H-purin-6-ylamino)-propyl]-3H-quinazolin-4-one; 5-fluoro-3-phenyl-2-[1-(9H-purin-6-ylamino)-propyl]-3H-quinazolin-4-one; 3-(2,6-difluoro-phenyl)-5-methyl-2-[1-(9H-purin-6-ylamino)-propyl]-3H-quinazolin-4-one; 6-fluoro-3-phenyl-2-[1-(9H-purin-6-ylamino)-ethyl]-3H-quinazolin-4-one; 3-(3,5-difluoro-phenyl)-5-methyl-2-[1-(9H-purin-6-ylamino)-ethyl]-3H-quinazolin-4-one; 5-fluoro-3-phenyl-2-[1-(9H-purin-6-ylamino)-ethyl]-3H-quinazolin-4-one; 3-(2,3-difluoro-phenyl)-5-methyl-2-[1-(9H-purin-6-ylamino)-ethyl]-3H-quinazolin-4-one; 5-methyl-3-phenyl-2-[1-(9H-purin-6-ylamino)-ethyl]-3H-quinazolin-4-one; 3-(3-chloro-phenyl)-5-methyl-2-[1-(9H-purin-6-ylamino)-ethyl]-3H-quinazolin-4-one; 5-methyl-3-phenyl-2-[(9H-purin-6-ylamino)-methyl]-3H-quinazolin-4-one; 2-[(2-amino-9H-purin-6-ylamino)-methyl]-3-(3,5-difluoro-phenyl)-5-methyl-3H-quinazolin-4-one; 3-{2-[(2-diethylamino-ethyl)-methyl-amino]-phenyl}-5-methyl-2-[(9H-purin-6-ylamino)-methyl]-3H-quinazolin-4-one; 5-chloro-3-(2-fluoro-phenyl)-2-[(9H-purin-6-ylamino)-methyl]-3H-quinazolin-4-one; 5-chloro-2-[(9H-purin-6-ylamino)-methyl]-3-o-tolyl-3H-quinazolin-4-one; 5-chloro-3-(2-chloro-phenyl)-2-[(9H-purin-6-ylamino)-methyl]-3H-quinazolin-4-one; 6-fluoro-3-(3-fluoro-phenyl)-2-[1-(9H-purin-6-ylamino)-ethyl]-3H-quinazolin-4-one; 2-[1-(2-amino-9H-purin-6-ylamino)-ethyl]-5-chloro-3-(3-fluoro-phenyl)-3H-quinazolin-4-one; 5-methyl-3-phenyl-2-[1-(9H-purin-6-ylamino)-propyl]-3H-quinazolin-4-one; 2-[1-(2-fluoro-9H-purin-6-ylamino)-ethyl]-5-methyl-3-phenyl-3H-quinazolin-4-one; 3-(2,6-difluoro-phenyl)-5-methyl-2-[1-(9H-purin-6-ylamino)-ethyl]-3H-quinazolin-4-one; 2-[1-(2-amino-9H-purin-6-ylamino)-ethyl]-3-(2,6-difluoro-phenyl)-5-methyl-3H-quinazolin-4-one; 3-(2,6-difluoro-phenyl)-2-[1-(2-fluoro-9H-purin-6-ylamino)-ethyl]-5-methyl-3H-quinazolin-4-one; 3-(2,6-difluoro-phenyl)-5-methyl-2-[1-(7H-pyrrolo[2,3-d]pyrimidin-4-ylamino)-ethyl]-3H-quinazolin-4-one; 2-[1-(2-amino-9H-purin-6-ylamino)-propyl]-5-methyl-3-phenyl-3H-quinazolin-4-one; 5-methyl-3-phenyl-2-[1-(7H-pyrrolo[2,3-d]pyrimidin-4-ylamino)-propyl]-3H-quinazolin-4-one; 2-[1-(2-fluoro-9h-purin-6-ylamino)-propyl]-5-methyl-3-phenyl-3h-quinazolin-4-one; 5-methyl-3-phenyl-2-[1-(9H-purin-6-ylamino)-ethyl]-3H-quinazolin-4-one; 2-[1-(2-amino-9H-purin-6-ylamino)-ethyl]-5-methyl-3-phenyl-3H-quinazolin-4-one; 2-[2-benzyloxy-1-(9H-purin-6-ylamino)-ethyl]-5-methyl-3-phenyl-3H-quinazolin-4-one; 2-[1-(2-amino-9H-purin-6-ylamino)-2-benzyloxy-ethyl]-5-methyl-3-phenyl-3H-quinazolin-4-one; 2-[2-benzyloxy-1-(7H-pyrrolo[2,3-d]pyrimidin-4-ylamino)-ethyl]-5-methyl-3-phenyl-3H-quinazolin-4-one; 2-[2-benzyloxy-1-(2-fluoro-9H-purin-6-ylamino)-ethyl]-5-methyl-3-phenyl-3H-quinazolin-4-one; 3-(4-fluoro-phenyl)-5-methyl-2-[1-(9H-purin-6-ylamino)-ethyl]-3H-quinazolin-4-one; 2-[1-(2-amino-9H-purin-6-ylamino)-ethyl]-3-(4-fluoro-phenyl)-5-methyl-3H-quinazolin-4-one; 3-(4-fluoro-phenyl)-2-[1-(2-fluoro-9H-purin-6-ylamino)-ethyl]-5-methyl-3H-quinazolin-4-one; 3-(4-fluoro-phenyl)-5-methyl-2-[1-(7H-pyrrolo[2,3-d]pyrimidin-4-ylamino)-ethyl]-3H-quinazolin-4-one; 5-methyl-3-phenyl-2-[1-(7H-pyrrolo[2,3-d]pyrimidin-4-ylamino)-ethyl]-3H-quinazolin-4-one; 3-(3-fluoro-phenyl)-5-methyl-2-[1-(9H-purin-6-ylamino)-ethyl]-3H-quinazolin-4-one; 2-[1-(2-amino-9H-purin-6-ylamino)-ethyl]-3-(3-fluoro-phenyl)-5-methyl-3H-quinazolin-4-one; 3-(3-fluoro-phenyl)-5-methyl-2-[1-(7H-pyrrolo[2,3-d]pyrimidin-4-ylamino)-ethyl]-3H-quinazolin-4-one; 5-methyl-3-phenyl-2-[1-(9H-purin-6-yl)-pyrrolidin-2-yl]-3H-quinazolin-4-one; 2-[2-hydroxy-1-(9H-purin-6-ylamino)-ethyl]-5-methyl-3-phenyl-3H-quinazolin-4-one; 5-methyl-3-phenyl-2-[phenyl-(9H-purin-6-ylamino)-methyl]-3H-quinazolin-4-one; 2-[(2-amino-9H-purin-6-ylamino)-phenyl-methyl]-5-methyl-3-phenyl-3H-quinazolin-4-one; 2-[(2-fluoro-9H-purin-6-ylamino)-phenyl-methyl]-5-methyl-3-phenyl-3H-quinazolin-4-one; 5-methyl-3-phenyl-2-[phenyl-(7H-pyrrolo[2,3-d]pyrimidin-4-ylamino)-methyl]-3H-quinazolin-4-one; 5-fluoro-3-phenyl-2-[1-(9H-purin-6-ylamino)-ethyl]-3H-quinazolin-4-one; 2-[1-(2-amino-9H-purin-6-ylamino)-ethyl]-5-fluoro-3-phenyl-3H-quinazolin-4-one; 2-[1-(2-amino-9H-purin-6-ylamino)-ethyl]-5-chloro-3-phenyl-3H-quinazolin-4-one; [5-(5-methyl-4-oxo-3-phenyl-3,4-dihydro-quinazolin-2-yl)-5-(9H-purin-6-ylamino)-pentyl]-carbamic acid benzyl ester; [5-(2-amino-9H-purin-6-ylamino)-5-(5-methyl-4-oxo-3-phenyl-3,4-dihydro-quinazolin-2-yl)-pentyl]-carbamic acid benzyl ester; [4-(5-methyl-4-oxo-3-phenyl-3,4-dihydro-quinazolin-2-yl)-4-(9H-purin-6-ylamino)-butyl]-carbamic acid benzyl ester; [4-(2-amino-9H-purin-6-ylamino)-4-(5-methyl-4-oxo-3-phenyl-3,4-dihydro-quinazolin-2-yl)-butyl]-carbamic acid benzyl ester; 3-phenyl-2-[1-(9H-purin-6-ylamino)-ethyl]-3H-quinazolin-4-one; 2-[5-amino-1-(9H-purin-6-ylamino)-pentyl]-5-methyl-3-phenyl-3H-quinazolin-4-one); 2-[5-amino-1-(2-amino-9H-purin-6-ylamino)-pentyl]-5-methyl-3-phenyl-3H-quinazolin-4-one; 2-[1-(2-amino-9H-purin-6-ylamino)-ethyl]-3-(2,6-Dimethyl-phenyl)-5-methyl-3H-quinazolin-4-one; 3-(2,6-dimethyl-phenyl)-5-methyl-2-[1-(9H-purin-6-ylamino)-ethyl]-3H-quinazolin-4-one; 5-morpholin-4-ylmethyl-3-phenyl-2-[1-(9H-purin-6-ylamino)-ethyl]-3H-quinazolin-4-one; 2-[1-(2-amino-9H-purin-6-ylamino)-ethyl]-5-morpholin-4ylmethyl-3-phenyl-3H-quinazolin-4-one; 2-[4-amino-1-(2-amino-9H-purin-6-ylamino)-butyl]-5-methyl-3-phenyl-3H-quinazolin-4-one; 6-fluoro-3-phenyl-2-[1-(9H-purin-6-ylamino)-ethyl]-3H-quinazolin-4-one; 2-[1-(2-amino-9H-purin-6-ylamino)-ethyl]-6-fluoro-3-phenyl-3H-quinazolin-4-one; 2-[2-tert-butoxy-1-(9H-purin-6-ylamino)-ethyl]-5-methyl-3-phenyl-3H-quinazolin-4-one; 3-(3-methyl-phenyl)-5-methyl-2-[1-(9H-purin-6-ylamino)-ethyl]-3H-quinazolin-4-one; 2-[1-(2-amino-9H-purin-6-ylamino)-ethyl]-3-(3-methyl-phenyl)-5-methyl-3H-quinazolin-4-one; 3-(3-chloro-phenyl)-5-methyl-2-[1-(9H-purin-6-ylamino)-ethyl]-3H-quinazolin-4-one; 2-[1-(2-amino-9H-purin-6-ylamino)-ethyl]-3-(3-chloro-phenyl)-5-methyl-3H-quinazolin-4-one; 2-[1-(2-amino-9H-purin-6-ylamino)-2-hydroxy-ethyl]-5-methyl-3-phenyl-3H-quinazolin-4-one; 2-[1-(2-amino-9H-purin-6-ylamino)-ethyl]-3-(3-fluoro-phenyl)-3H-quinazolin-4-one; 2-[1-(2-amino-9H-purin-6-ylamino)-ethyl]-3-(2,6-difluoro-phenyl)-3H-quinazolin-4-one; 2-[1-(2-amino-9H-purin-6-ylamino)-propyl]-5-fluoro-3-phenyl-3H-quinazolin-4-one; 5-chloro-3-(3-fluoro-phenyl)-2-[1-(9H-purin-6-ylamino)-ethyl]-3H-quinazolin-4-one; 2-[1-(2-amino-9H-purin-6-ylamino)-ethyl]-5-chloro-3-(3-fluoro-phenyl)-3H-quinazolin-4-one; 3-phenyl-2-[1-(9H-purin-6-ylamino)-ethyl]-5-trifluoromethyl-3H-quinazolin-4-one; 3-(2,6-difluoro-phenyl)-5-methyl-2-[1-(9H-purin-6-ylamino)-propyl]-3H-quinazolin-4-one; 3-(2,6-difluoro-phenyl)-5-methyl-2-[1-(9H-purin-6-ylamino)-ethyl]-3H-quinazolin-4-one; 2-[1-(2-amino-9H-purin-6-ylamino)-propyl]-3-(2,6-difluoro-phenyl)-5-methyl-3H-quinazolin-4-one; 2-[1-(2-amino-9H-purin-6-ylamino)-ethyl]-3-(2,6-difluoro-phenyl)-5-methyl-3H-quinazolin-4-one; 3-(3,5-dichloro-phenyl)-5-methyl-2-[1-(9H-purin-6-ylamino)-ethyl]-3H-quinazolin-4-one; 3-(2,6-dichloro-phenyl)-5-methyl-2-[1-(9H-purin-6-ylamino)-ethyl]-3H-quinazolin-4-one; 2-[1-(2-amino-9H-purin-6-ylamino)-ethyl]-3-(2,6-dichloro-phenyl)-5-methyl-3H-quinazolin-4-one; 5-chloro-3-phenyl-2-[1-(9H-purin-6-ylamino)-propyl]-3H-quinazolin-4-one; 2-[1-(2-amino-9H-purin-6-ylamino)-propyl]-5-chloro-3-phenyl-3H-quinazolin-4-one; 5-methyl-3-phenyl-2-[1-(9H-purin-6-ylamino)-butyl]-3H-quinazolin-4-one; 2-[1-(2-amino-9H-purin-6-ylamino)-butyl]-5-methyl-3-phenyl-3H-quinazolin-4-one; 2-[1-(2-amino-9H-purin-6-ylamino)-ethyl]-3-(3,5-dichloro-phenyl)-5-methyl-3H-quinazolin-4-one; 5-methyl-3-(3-morpholin-4-ylmethyl-phenyl)-2-[1-(9H-purin-6-ylamino)-ethyl]-3H-quinazolin-4-one; 2-[1-(2-amino-9H-purin-6-ylamino)-ethyl]-5-methyl-3-(3-morpholin-4-ylmethyl-phenyl)-3H-quinazolin-4-one; 2-[1-(5-bromo-7H-pyrrolo[2,3-d]pyrimidin-4-ylamino)-ethyl]-5-methyl-3-phenyl-3H-quinazolin-4-one; 5-methyl-2-[1-(5-methyl-7H-pyrrolo[2,3-d]pyrimidin-4-ylamino)-ethyl]-3-phenyl-3H-quinazolin-4-one; 2-[1-(5-fluoro-7H-pyrrolo[2,3-d]pyrimidin-4-ylamino)-ethyl]-5-methyl-3-phenyl-3H-quinazolin-4-one; 2-[2-hydroxy-1-(9H-purin-6-ylamino)-ethyl]-3-phenyl-3H-quinazolin-4-one; 3-(3,5-difluoro-phenyl)-5-methyl-2-[1-(9H-purin-6-ylamino)-propyl]-3H-quinazolin-4-one; 2-[1-(2-amino-9H-purin-6-ylamino)-propyl]-3-(3,5-difluoro-phenyl)-5-methyl-3H-quinazolin-4-one; 3-(3,5-difluoro-phenyl)-2-[1-(9H-purin-6-ylamino)-ethyl]-3H-quinazolin-4-one; 2-[1-(5-bromo-7H-pyrrolo[2,3-d]pyrimidin-4-ylamino)-ethyl]-3-(3-fluoro-phenyl)-5-methyl-3H-quinazolin-4-one; 3-(3-fluoro-phenyl)-5-methyl-2-[1-(5-methyl-7H-pyrrolo[2,3-d]pyrimidin-4-ylamino)-ethyl]-3H-quinazolin-4-one; 3-phenyl-2-[1-(9H-purin-6-ylamino)-propyl]-3H-quinazolin-4-one; 2-[1-(2-amino-9H-purin-6-ylamino)-ethyl]-3-(3,5-difluoro-phenyl)-3H-quinazolin-4-one; 2-[1-(2-amino-9H-purin-6-ylamino)-propyl]-3-phenyl-3H-quinazolin-4-one; 6,7-difluoro-3-phenyl-2-[1-(9H-purin-6-ylamino)-ethyl]-3H-quinazolin-4-one; 6-fluoro-3-(3-fluoro-phenyl)-2-[1-(9H-purin-6-ylamino)-ethyl]-3H-quinazolin-4-one; 2-[4-diethylamino-1-(9H-purin-6-ylamino)-butyl]-5-methyl-3-phenyl-3H-quinazolin-4-one; 5-fluoro-3-phenyl-2-[1-(9H-purin-6-ylamino)-propyl]-3H-quinazolin-4-one; 3-phenyl-2-[1-(9H-purin-6-ylamino)-ethyl]-3H-quinazolin-4-one; 6-fluoro-3-phenyl-2-[1-(9H-purin-6-ylamino)-ethyl]-3H-quinazolin-4-one; 3-(3,5-difluoro-phenyl)-5-methyl-2-[1-(9H-purin-6-ylamino)-ethyl]-3H-quinazolin-4-one; 5-fluoro-2-[1-(2-fluoro-9H-purin-6-ylamino)-ethyl]-3-phenyl-3H-quinazolin-4-one; 3-(3-fluoro-phenyl)-2-[1-(9H-purin-6-ylamino)-ethyl]-3H-quinazolin-4-one; 5-chloro-3-(3,5-difluoro-phenyl)-2-[1-(9H-purin-6-ylamino)-propyl]-3H-quinazolin-4-one; 3-(2,6-difluoro-phenyl)-5-methyl-2-[1-(9H-purin-6-ylamino)-ethyl]-3H-quinazolin-4-one; 3-(2,6-difluoro-phenyl)-2-[1-(9H-purin-6-ylamino)-ethyl]-3H-quinazolin-4-one; 5-Methyl-3-phenyl-2-[3,3,3-trifluoro-1-(9H-purin-6-ylamino)-propyl]-3H-quinazolin-4-one; 3-(3-hydroxy-phenyl)-5-methyl-2-[1-(9H-purin-6-ylamino)-ethyl]-3H-quinazolin-4-one; 3-(3-methoxy-phenyl)-5-methyl-2-{1-[9H-purin-6-ylamino]-ethyl}-3H-quinazolin-4-one; 3-[3-(2-dimethylamino-ethoxy)-phenyl]-5-methyl-2-{1-[9H-purin-6-ylamino]-ethyl}-3H-quinazolin-4-one; 3-(3-cyclopropylmethoxy-phenyl)-5-methyl-2-{1-[9H-purin-6-ylamino]-ethyl}-3H-quinazolin-4-one; 5-methyl-3-(3-prop-2-ynyloxy-phenyl)-2-{1-[9H-purin-6-ylamino]-ethyl}-3H-quinazolin-4-one; 2-{1-[2-amino-9H-purin-6-ylamino]ethyl}-3-(3-hydroxyphenyl)-5-methyl-3H-quinazolin-4-one; 2-{1-[2-amino-9H-purin-6-ylamino]ethyl}-3-(3-methoxyphenyl)-5-methyl-3H-quinazolin-4-one; 2-{1-[2-amino-9H-purin-6-ylamino]ethyl}-3-(3-cyclopropylmethoxy-phenyl)-5-methyl-3H-quinazolin-4-one; 2-{1-[2-amino-9H-purin-6-ylamino]ethyl}-5-methyl-3-(3-prop-2-ynyloxy-phenyl)-3H-quinazolin-4-one; 3-(3-ethynyl-phenyl)-5-methyl-2-[1-(9H-purin-6-ylamino)-ethyl]-3H-quinazolin-4-one; 3-{5-methyl-4-oxo-2-[1-(9H-purin-6-ylamino)-ethyl]-4H-quinazolin-3-yl}-benzonitrile; 3-{5-methyl-4-oxo-2-{1-[9H-purin-6-ylamino)-ethyl]-4H-quinazolin-3-yl}-benzamide; 3-(3-acetyl-phenyl)-5-methyl-2-{1-[9H-purin-6-ylamino]-ethyl}-3H-quinazolin-4-one; 2-(3-(5-methyl-4-oxo-2-{1-[9H-purin-6-ylamino]-ethyl}-4H-quinazolin-3-yl-phenoxy acetamide; 5-methyl-2-{1-[9H-purin-6-ylamino]-ethyl}-3-[3-(tetrahydropuran-4-yloxy)-phenyl]-3H-quinazolin-4-one; 3-[3-(2-methoxy-ethoxy)-phenyl]-5-methyl-2-[1-(9H-purin-6-ylamino)-ethyl]-3H-quinazolin-4-one; 6-fluoro-2-[1-(9H-purin-6-ylamino)ethyl]-3-[3-(tetrahydro-pyran-4-yloxy)-phenyl]-3H-quinazolin-4-one; 3-[3-(3-dimethylamino-propoxy)-phenyl]-5-methyl-2-[1-(9H-purin-6-ylamino)-ethyl]-3H-quinazolin-4-one; 2-[1-(2-amino-9H-purin-6-ylamino)-ethyl]-3-(3-ethynyl-phenyl)-5-methyl-3H-quinazolin-4-one; 3-{2-[1-(2-amino-9H-purin-6-ylamino)-ethyl]-5-methyl-4-oxo-4H-quinazolin-3-yl}-benzonitrile; 3-{2-[1-(2-amino-9H-purin-6-ylamino)-ethyl]-5-methyl-4-oxo-4H-quinazolin-3-yl}-benzamide; 3-{2-[1-(2-amino-9H-purin-6-ylamino)-ethyl]-5-methyl-4-oxo-4H-quinazolin-3-yl}-benzamide; 5-methyl-3-(3-morpholin-4-yl-phenyl)-2-[1-(9H-purin-6-ylamino)-ethyl]-3H-quinazolin-4-one; 2-[1-(2-amino-9H-purin-6-ylamino)-ethyl]-5-methyl-3-(3-morpholin-4-yl-phenyl)-3H-quinazolin-4-one; 2-[1-(2-amino-9H-purin-6-ylamino)-ethyl]-3-[3-(2-methoxy-ethoxy)-phenyl]-5-methyl-3H-quinazolin-4-one; 2-[1-(2-amino-9H-purin-6-ylamino)-ethyl]-3-[3-(2-dimethylamino-ethoxy)-phenyl]-5-methyl-3H-quinazolin-4-one; 2-[1-(2-amino-9H-purin-6-ylamino)-but-3-ynyl]-5-methyl-3-phenyl-3H-quinazolin-4-one; 2-[1-(2-amino-9H-purin-6-ylamino)-but-3-ynyl]-5-methyl-3-phenyl-3H-quinazolin-4-one; 5-chloro-3-(3,5-difluoro-phenyl)-2-[1-(9H-purin-6-ylamino)-ethyl]-3H-quinazolin-4-one; 2-[1-(2-amino-9H-purin-6-ylamino)-propyl]-5-chloro-3-(3,5-difluoro-phenyl)-3H-quinazolin-4-one; 2-[1-(2-amino-9H-purin-6-ylamino)-ethyl]-5-chloro-3-(3,5-difluoro-phenyl)-3H-quinazolin-4-one; 3-(3,5-difluoro-phenyl)-6-fluoro-2-[1-(9H-purin-6-ylamino)-ethyl]-3H-quinazolin-4-one; 5-chloro-3-(2,6-difluoro-phenyl)-2-[1-(9H-purin-6-ylamino)-propyl]-3H-quinazolin-4-one; 2-[1-(2-amino-9H-purin-6-ylamino)-propyl]-5-chloro-3-(2,6-difluoro-phenyl)-3H-quinazolin-4-one; 5-methyl-3-phenyl-2-[1-(9H-purin-6-yloxy)-ethyl]-3H-quinazolin-4-one; and,
pharmaceutically acceptable salts and solvates thereof.

17. A method for treating and/or preventing leukemia, comprising:
selectively inhibiting phosphoinositide 3-kinase delta (PI3Kδ) activity in leukemic cells, thereby treating and/or preventing leukemia.

18. The method of claim 17, wherein inhibiting comprises administering an amount of a PI3Kδ selective inhibitor effective to inhibit PI3Kδ activity of leukemic cells.

19. The method of claim 17, wherein said inhibiting is in vitro.

20. The method of claim 17, wherein said inhibiting is performed in an individual in need thereof.

21. The method of claim 20, wherein the PI3K pathway is constitutively activated in the leukemic cells.

22. The method of claim 17, wherein the leukemia is selected from the group consisting of acute lymphoblastic leukemia; acute myeloid leukemia; chronic lymphocytic leukemia; chronic myelogenous leukemia; and, hairy cell leukemia.

23. The method of claim 17, further comprising administering a mammalian target of rapamycin (mTOR) inhibitor.

24. The method of claim 23, wherein the mTOR inhibitor is selected from the group consisting of rapamycin, FK506, cyclosporine A (CsA), and everolimus.

25. The method of claim 18, wherein the PI3Kδ selective inhibitor is administered in an amount effective to inhibit Akt phosphorylation.

26. The method of claim 18, wherein the PI3Kδ selective inhibitor is administered in an amount effective to inhibit FOXO3a phosphorylation.

27. The method of claim 18, wherein the PI3Kδ selective inhibitor is administered in an amount effective to inhibit GAB1 phosphorylation.

28. The method of claim 18, wherein the PI3Kδ selective inhibitor is administered in an amount effective to inhibit GAB2 phosphorylation.

29. The method of claim 18, wherein the PI3Kδ selective inhibitor is a compound having formula (I) or pharmaceutically acceptable salts and solvates thereof: 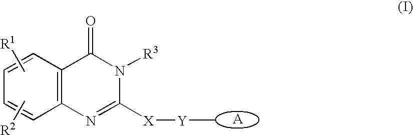
wherein A is an optionally substituted monocyclic or bicyclic ring system containing at least two nitrogen atoms, and at least one ring of the system is aromatic;
X is selected from the group consisting of C(R^{b})₂, CH₂CHR^{b}, and CH═C(R^{b});
Y is selected from the group consisting of null, S, SO, SO₂, NH, O, C(═O), OC(═O), C(═O)O, and NHC(═O)CH₂S;
R¹ and R², independently, are selected from the group consisting of hydrogen, C₁₋₆alkyl, aryl, heteroaryl, halo, NHC(═O)C₁₋₃alkyleneN(R^{a})₂, NO₂, OR^{a}, CF₃, OCF₃, N(R^{a})₂, CN, OC(═O)R^{a}, C(═O)R^{a}, C(═O)OR^{a}, arylOR^{b}, Het, NR^{a}C(═O)C₁₋₃alkyleneC(═O)OR^{a}, arylOC₁₋₃alkyleneN(R^{b})₂, arylOC(═O)R^{a}, C₁₋₄alkyleneC(═O)OR^{a}, OC₁₋₄alkyleneC(═O)OR^{a}, C₁₋₄alkyleneOC₁₋₄alkyleneC(═O)OR^{a}, C(═O)NR^{a}SO₂R^{a}, C₁₋₄alkyleneN(R^{a})₂, C₂₋₆alkenyleneN(R^{a})₂, C(═O)NR^{a}C₁₋₄alkyleneOR^{a}, C(═O)NR^{a}C₁₋₄alkyleneHet, OC₂₋₄alkyleneN(R^{a})₂, OC₁₋₄alkyleneCH(OR^{b})CH₂N(R^{a})₂, OC₁₋₄alkyleneHet, OC₂₋₄alkyleneOR^{a}, OC₂₋₄alkyleneNR^{a}C(═O)OR^{a}, NR^{a}C₁₋₄alkyleneN(R^{a})₂, NR^{a}C(═O)R^{a}, NR^{a}C(═O)N(R^{a})₂, N(SO₂C₁₋₄alkyl)₂, NR^{a}(SO₂C₁₋₄alkyl), SO₂N(R^{a})₂, OSO₂CF₃, C₁₋₃alkylenearyl, C₁₋₄alkyleneHet, C₁₋₆alkyleneOR^{b}, C₁₋₃alkyleneN(R^{a})₂, C(═O)N(R^{a})₂, NHC(═O)C₁₋₁₃alkylenearyl, C₃₋₈cycloalkyl, C₃₋₈heterocycloalkyl, arylOC₁₋₃alkyleneN(R^{a})₂, arylOC(═O)R^{b}, NHC(═O)C₁₋₃alkyleneC₃₋₈heterocycloalkyl, NHC(═O)C₁₋₃alkyleneHet, OC₁₋₄alkyleneOC₁₋₄alkyleneC(═O)OR^{b}, C(═O)C₁₋₄alkyleneHet, and NHC(═O)haloC₁₋₆alkyl;
or R¹ and R² are taken together to form a 3- or 4-membered alkylene or alkenylene chain component of a 5- or 6-membered ring, optionally containing at least one heteroatom;
R³ is selected from the group consisting of optionally substituted hydrogen, C₁₋₆alkyl, C₃₋₈cycloalkyl, C₃₋₈heterocycloalkyl, C₁₋₄alkylenecycloalkyl, C₂₋₆alkenyl, C₁₋₃alkylenearyl, arylC₁₋₃alkyl, C(═O)R^{a}, aryl, heteroaryl, C(═O)OR^{a}, C(═O)N(R^{a})₂, C(═S)N(R^{a})₂, SO₂R^{a}, SO₂N(R^{a})₂, S(═O)R^{a}, S(═O)N(R^{a})₂, C(═O)NR^{a}C₁₋₄alkyleneOR^{a}, C(═O)NR^{a}C₁₋₄alkyleneHet, C(═O)C₁₋₄alkylenearyl, C(═O)C₁₋₄alkyleneheteroaryl, C₁₋₄alkylenearyl optionally substituted with one or more of halo, SO₂N(R^{a})₂, N(R^{a})₂, C(═O)OR^{a}, NR^{a}SO₂CF₃, CN, NO₂, C(═O)R^{a}, OR^{a}, C₁₋₄alkyleneN(R^{a})₂, and OC₁₋₄alkyleneN(R^{a})₂, C₁₋₄alkyleneheteroaryl, C₁₋₄alkyleneHet, C₁₋₄alkyleneC(═O)C₁₋₄alkylenearyl, C₁₋₄alkyleneC(═O)C₁₋₄alkyleneheteroaryl, C₁₋₄alkyleneC(═O)Het, C₁₋₄alkyleneC(═O)N(R^{a})₂, C₁₋₄alkyleneOR^{a}, C₁₋₄alkyleneNR^{a}C(═O)R^{a}, C₁₋₄alkyleneOC₁₋₄alkyleneOR^{a}, C₁₋₄alkyleneN(R^{a})₂, C₁₋₄alkyleneC(═O)OR^{a}, and C₁₋₄alkyleneOC₁₋₄alkyleneC(═O)OR^{a};
R^{a} is selected from the group consisting of hydrogen, C₁₋₆alkyl, C₃₋₈cycloalkyl, C₃₋₈heterocycloalkyl, C₁₋₃alkyleneN(R^{c})₂, aryl, arylC₁₋₃alkyl, C₁₋₃alkylenearyl, heteroaryl, heteroarylC₁₋₃alkyl, and C₁₋₃alkyleneheteroaryl;
or two R^{a} groups are taken together to form a 5- or 6-membered ring, optionally containing at least one heteroatom;
R^{b} is selected from the group consisting of hydrogen, C₁₋₆alkyl, heteroC₁₋₃alkyl, C₁₋₃alkyleneheteroC₁₋₃alkyl, arylheteroC₁₋₃alkyl, aryl, heteroaryl, arylC₁₋₃alkyl, heteroarylC₁₋₃alkyl, C₁₋₃alkylenearyl, and C₁₋₃alkyleneheteroaryl;
R^{c} is selected from the group consisting of hydrogen, C₁₋₆alkyl, C₃₋₈cycloalkyl, aryl, and heteroaryl; and,
Het is a 5- or 6-membered heterocyclic ring, saturated or partially or fully unsaturated, containing at least one heteroatom selected from the group consisting of oxygen, nitrogen, and sulfur, and optionally substituted with C₁₋₄alkyl or C(═O)OR^{a}.

30. The method of claim 18, wherein the PI3Kδ selective inhibitor is selected from the group consisting of:
2-(6-aminopurin-9-ylmethyl)-3-(2-chlorophenyl)-6,7-dimethoxy-3H-quinazolin-4-one; 2-(6-aminopurin-o-ylmethyl)-6-bromo-3-(2-chlorophenyl)-3H-quinazolin-4-one; 2-(6-aminopurin-o-ylmethyl)-3-(2-chlorophenyl)-7-fluoro-3H-quinazolin-4-one; 2-(6-aminopurin-9-ylmethyl)-6-chloro-3-(2-chlorophenyl)-3H-quinazolin-4-one; 2-(6-aminopurin-9-ylmethyl)-3-(2-chlorophenyl)-5-fluoro-3H-quinazolin-4-one; 2-(6-aminopurin-o-ylmethyl)-5-chloro-3-(2-chloro-phenyl)-3H-quinazolin-4-one; 2-(6-aminopurin-9-ylmethyl)-3-(2-chlorophenyl)-5-methyl-3H-quinazolin-4-one; 2-(6-aminopurin-9-ylmethyl)-8-chloro-3-(2-chlorophenyl)-3H-quinazolin-4-one; 2-(6-aminopurin-9-ylmethyl)-3-biphenyl-2-yl-5-chloro-3H-quinazolin-4-one; 5-chloro-2-(9H-purin-6-ylsulfanylmethyl)-3-o-tolyl-3H-quinazolin-4-one; 5-chloro-3-(2-fluorophenyl)-2-(9H-purin-6-yl-sulfanylmethyl)-3H-quinazolin-4-one; 2-(6-aminopurin-9-ylmethyl)-5-chloro-3-(2-fluorophenyl)-3H-quinazolin-4-one; 3-biphenyl-2-yl-5-chloro-2-(9H-purin-6-ylsulfanylmethyl)-3H-quinazolin-4-one; 5-chloro-3-(2-methoxyphenyl)-2-(9H-purin-6-yl-sulfanylmethyl)-3H-quinazolin-4-one; 3-(2-chlorophenyl)-5-fluoro-2-(9H-purin-6-yl-sulfanylmethyl)-3H-quinazolin-4-one; 3-(2-chlorophenyl)-6,7-dimethoxy-2-(9H-purin-6-yl-sulfanylmethyl)-3H-quinazolin-4-one; 6-bromo-3-(2-chlorophenyl)-2-(9H-purin-6-yl-sulfanylmethyl)-3H-quinazolin-4-one; 3-(2-chlorophenyl)-8-trifluoromethyl-2-(9H-purin-6-ylsulfanylmethyl)-3H-quinazolin-4-one; 3-(2-chlorophenyl)-2-(9H-purin-6-ylsulfanylmethyl)-3H-benzo[g]quinazolin-4-one; 6-chloro-3-(2-chlorophenyl)-2-(9H-purin-6-yl-sulfanylmethyl)-3H-quinazolin-4-one; 8-chloro-3-(2-chlorophenyl)-2-(9H-purin-6-yl-sulfanylmethyl)-3H-quinazolin-4-one; 3-(2-chlorophenyl)-7-fluoro-2-(9H-purin-6-yl-sulfanylmethyl)-3H-quinazolin-4-one; 3-(2-chlorophenyl)-7-nitro-2-(9H-purin-6-yl-sulfanylmethyl)-3H-quinazolin-4-one; 3-(2-chlorophenyl)-6-hydroxy-2-(9H-purin-6-yl-sulfanylmethyl)-3H-quinazolin-4-one; 5-chloro-3-(2-chlorophenyl)-2-(9H-purin-6-yl-sulfanylmethyl)-3H-quinazolin-4-one; 3-(2-chlorophenyl)-5-methyl-2-(9H-purin-6-yl-sulfanylmethyl)-3H-quinazolin-4-one; 3-(2-chlorophenyl)-6,7-difluoro-2-(9H-purin-6-yl-sulfanylmethyl)-3H-quinazolin-4-one; 3-(2-chlorophenyl)-6-fluoro-2-(9H-purin-6-yl-sulfanylmethyl)-3H-quinazolin-4-one; 2-(6-aminopurin-9-ylmethyl)-3-(2-isopropylphenyl)-5-methyl-3H-quinazolin-4-one; 2-(6-aminopurin-9-ylmethyl)-5-methyl-3-o-tolyl-3H-quinazolin-4-one; 3-(2-fluorophenyl)-5-methyl-2-(9H-purin-6-yl-sulfanylmethyl)-3H-quinazolin-4-one; 2-(6-aminopurin-9-ylmethyl)-5-chloro-3-o-tolyl-3H-quinazolin-4-one; 2-(6-aminopurin-9-ylmethyl)-5-chloro-3-(2-methoxy-phenyl)-3H-quinazolin-4-one; 2-(2-amino-9H-purin-6-ylsulfanylmethyl)-3-cyclopropyl-5-methyl-3H-quinazolin-4-one; 3-cyclopropylmethyl-5-methyl-2-(9H-purin-6-ylsulfanylmethyl)-3H-quinazolin-4-one; 2-(6-aminopurin-9-ylmethyl)-3-cyclopropylmethyl-5-methyl-3H-quinazolin-4-one; 2-(2-amino-9H-purin-6-ylsulfanylmethyl)-3-cyclopropylmethyl-5-methyl-3H-quinazolin-4-one; 5-methyl-3-phenethyl-2-(9H-purin-6-ylsulfanylmethyl)-3H-quinazolin-4-one; 2-(2-amino-9H-purin-6-ylsulfanylmethyl)-5-methyl-3-phenethyl-3H-quinazolin-4-one; 3-cyclopentyl-5-methyl-2-(9H-purin-6-ylsulfanylmethyl)-3H-quinazolin-4-one; 2-(6-aminopurin-9-ylmethyl)-3-cyclopentyl-5-methyl-3H-quinazolin-4-one; 3-(2-chloropyridin-3-yl)-5-methyl-2-(9H-purin-6-ylsulfanylmethyl)-3H-quinazolin-4-one; 2-(6-aminopurin-9-ylmethyl)-3-(2-chloropyridin-3-yl)-5-methyl-3H-quinazolin-4-one; 3-methyl-4-[5-methyl-4-oxo-2-(9H-purin-6-ylsulfanylmethyl)-4H-quinazolin-3-yl]-benzoic acid; 3-cyclopropyl-5-methyl-2-(9H-purin-6-ylsulfanylmethyl)-3H-quinazolin-4-one; 2-(6-aminopurin-9-ylmethyl)-3-cyclopropyl-5-methyl-3H-quinazolin-4-one; 5-methyl-3-(4-nitrobenzyl)-2-(9H-purin-6-ylsulfanylmethyl)-3H-quinazolin-4-one; 3-cyclohexyl-5-methyl-2-(9H-purin-6-ylsulfanylmethyl)-3H-quinazolin-4-one; 2-(6-aminopurin-9-ylmethyl)-3-cyclohexyl-5-methyl-3H-quinazolin-4-one; 2-(2-amino-9H-purin-6-ylsulfanylmethyl)-3-cyclo-hexyl-5-methyl-3H-quinazolin-4-one; 5-methyl-3-(E-2-phenylcyclopropyl)-2-(9H-purin-6-ylsulfanylmethyl)-3H-quinazolin-4-one; 3-(2-chlorophenyl)-5-fluoro-2-[(9H-purin-6-ylamino)methyl]-3H-quinazolin-4-one; 2-[(2-amino-9H-purin-6-ylamino)methyl]-3-(2-chlorophenyl)-5-fluoro-3H-quinazolin-4-one; 5-methyl-2-[(9H-purin-6-ylamino)methyl]-3-o-tolyl-3H-quinazolin-4-one; 2-[(2-amino-9H-purin-6-ylamino)methyl]-5-methyl-3-o-tolyl-3H-quinazolin-4-one; 2-[(2-fluoro-9H-purin-6-ylamino)methyl]-5-methyl-3-o-tolyl-3H-quinazolin-4-one; (2-chlorophenyl)-dimethylamino-(9H-purin-6-ylsulfanylmethyl)-3H-quinazolin-4-one; 5-(2-benzyloxyethoxy)-3-(2-chlorophenyl)-2-(9H-purin-6-ylsulfanylmethyl)-3H-quinazolin-4-one; 6-aminopurine-9-carboxylic acid 3-(2-chlorophenyl)-5-fluoro-4-oxo-3,4-dihydro-quinazolin-2-ylmethyl ester; N-[3-(2-chlorophenyl)-5-fluoro-4-oxo-3,4-dihydro-quinazolin-2-ylmethyl]-2-(9H-purin-6-ylsulfanyl)-acetamide; 2-[1-(2-fluoro-9H-purin-6-ylamino)ethyl]-5-methyl-3-o-tolyl-3H-quinazolin-4-one; 5-methyl-2-[1-(9H-purin-6-ylamino)ethyl]-3-o-tolyl-3H-quinazolin-4-one; 2-(6-dimethylaminopurin-9-ylmethyl)-5-methyl-3-o-tolyl-3H-quinazolin-4-one; 5-methyl-2-(2-methyl-6-oxo-1,6-dihydro-purin-7-ylmethyl)-3-o-tolyl-3H-quinazolin-4-one; 5-methyl-2-(2-methyl-6-oxo-1,6-dihydro-purin-9-ylmethyl)-3-o-tolyl-3H-quinazolin-4-one; 2-(amino-dimethylaminopurin-9-ylmethyl)-5-methyl-3-o-tolyl-3H-quinazolin-4-one; 2-(2-amino-9H-purin-6-ylsulfanylmethyl)-5-methyl-3-o-tolyl-3H-quinazolin-4-one; 2-(4-amino-1,3,5-triazin-2-ylsulfanylmethyl)-5-methyl-3-o-tolyl-3H-quinazolin-4-one; 5-methyl-2-(7-methyl-7H-purin-6-ylsulfanylmethyl)-3-o-tolyl-3H-quinazolin-4-one; 5-methyl-2-(2-oxo-1,2-dihydro-pyrimidin-4-ylsulfanylmethyl)-3-o-tolyl-3H-quinazolin-4-one; 5-methyl-2-purin-7-ylmethyl-3-o-tolyl-3H-quinazolin-4-one; 5-methyl-2-purin-9-ylmethyl-3-o-tolyl-3H-quinazolin-4-one; 5-methyl-2-(9-methyl-9H-purin-6-ylsulfanylmethyl)-3-o-tolyl-3H-quinazolin-4-one; 2-(2,6-diamino-pyrimidin-4-ylsulfanylmethyl)-5-methyl-3-o-tolyl-3H-quinazolin-4-one; 5-methyl-2-(5-methyl-[1,2,4]triazolo[1,5-a]pyrimidin-7-ylsulfanylmethyl)-3-o-tolyl-3H-quinazolin-4-one; 5-methyl-2-(2-methylsulfanyl-9H-purin-6-ylsulfanylmethyl)-3-o-tolyl-3H-quinazolin-4-one; 2-(2-hydroxy-9H-purin-6-ylsulfanylmethyl)-5-methyl-3-o-tolyl-3H-quinazolin-4-one; 5-methyl-2-(1-methyl-1H-imidazol-2-ylsulfanylmethyl)-3-o-tolyl-3H-quinazolin-4-one; 5-methyl-3-o-tolyl-2-(1H-[1,2,4]triazol-3-ylsulfanylmethyl)-3H-quinazolin-4-one; 2-(2-amino-6-chloro-purin-9-ylmethyl)-5-methyl-3-o-tolyl-3H-quinazolin-4-one; 2-(6-aminopurin-7-ylmethyl)-5-methyl-3-o-tolyl-3H-quinazolin-4-one; 2-(7-amino-1,2,3-triazolo[4,5-d]pyrimidin-3-yl-methyl)-5-methyl-3-o-tolyl-3H-quinazolin-4-one; 2-(7-amino-1,2,3-triazolo[4,5-d]pyrimidin-1-yl-methyl)-5-methyl-3-o-tolyl-3H-quinazolin-4-one; 2-(6-amino-9H-purin-2-ylsulfanylmethyl)-5-methyl-3-o-tolyl-3H-quinazolin-4-one; 2-(2-amino-6-ethylamino-pyrimidin-4-ylsulfanylmethyl)-5-methyl-3-o-tolyl-3H-quinazolin-4-one; 2-(3-amino-5-methylsulfanyl-1,2,4-triazol-1-yl-methyl)-5-methyl-3-o-tolyl-3H-quinazolin-4-one; 2-(5-amino-3-methylsulfanyl-1,2,4-triazol-1-ylmethyl)-5-methyl-3-o-tolyl-3H-quinazolin-4-one; 5-methyl-2-(6-methylaminopurin-9-ylmethyl)-3-o-tolyl-3H-quinazolin-4-one; 2-(6-benzylaminopurin-9-ylmethyl)-5-methyl-3-o-tolyl-3H-quinazolin-4-one; 2-(2,6-diaminopurin-9-ylmethyl)-5-methyl-3-o-tolyl-3H-quinazolin-4-one; 5-methyl-2-(9H-purin-6-ylsulfanylmethyl)-3-o-tolyl-3H-quinazolin-4-one; 3-isobutyl-5-methyl-2-(9H-purin-6-ylsulfanylmethyl)-3H-quinazolin-4-one; N-{2-[5-Methyl-4-oxo-2-(9H-purin-6-ylsulfanylmethyl)-4H-quinazolin-3-yl]-phenyl}acetamide; 5-methyl-3-(E-2-methyl-cyclohexyl)-2-(9H-purin-6-ylsulfanylmethyl)-3H-quinazolin-4-one; 2-[5-methyl-4-oxo-2-(9H-purin-6-ylsulfanylmethyl)-4H-quinazolin-3-yl]-benzoic acid; 3-{2-[(2-dimethylaminoethyl)methylamino]phenyl}-5-methyl-2-(9H-purin-6-ylsulfanylmethyl)-3H-quin-azolin-4-one; 3-(2-chlorophenyl)-5-methoxy-2-(9H-purin-6-ylsulfanylmethyl)-3H-quinazolin-4-one; 3-(2-chlorophenyl)-5-(2-morpholin-4-yl-ethylamino)-2-(9H-purin-6-ylsulfanylmethyl)-3H-quinazolin-4-one; 3-benzyl-5-methoxy-2-(9H-purin-6-ylsulfanylmethyl)-3H-quinazolin-4-one; 2-(6-aminopurin-9-ylmethyl)-3-(2-benzyloxyphenyl)-5-methyl-3H-quinazolin-4-one; 2-(6-aminopurin-9-ylmethyl)-3-(2-hydroxyphenyl)-5-methyl-3H-quinazolin-4-one; 2-(1-(2-amino-9H-purin-6-ylamino)ethyl)-5-methyl-3-o-tolyl-3H-quinazolin-4-one; 5-methyl-2-[1-(9H-purin-6-ylamino)propyl]-3-o-tolyl-3H-quinazolin-4-one; 2-(1-(2-fluoro-9H-purin-6-ylamino)propyl)-5-methyl-3-o-tolyl-3H-quinazolin-4-one; 2-(1-(2-amino-9H-purin-6-ylamino)propyl)-5-methyl-3-o-tolyl-3H-quinazolin-4-one; 2-(2-benzyloxy-1-(9H-purin-6-ylamino)ethyl)-5-methyl-3-o-tolyl-3H-quinazolin-4-one; 2-(6-aminopurin-9-ylmethyl)-5-methyl-3-{2-(2-(1-methylpyrrolidin-2-yl)-ethoxy)-phenyl}-3H-quinazolin-4-one; 2-(6-aminopurin-9-ylmethyl)-3-(2-(3-dimethylamino-propoxy)-phenyl)-5-methyl-3H-quinazolin-4-one; 2-(6-aminopurin-9-ylmethyl)-5-methyl-3-(2-prop-2-ynyloxyphenyl)-3H-quinazolin-4-one; 2-{2-(1-(6-aminopurin-9-ylmethyl)-5-methyl-4-oxo-4H-quinazolin-3-yl]-phenoxy}-acetamide; 2-[(6-aminopurin-9-yl)methyl]-5-methyl-3-o-tolyl-3-hydroquinazolin-4-one; 3-(3,5-difluorophenyl)-5-methyl-2-[(purin-6-ylamino)methyl]-3-hydroquinazolin-4-one; 3-(2,6-dichlorophenyl)-5-methyl-2-[(purin-6-ylamino)methyl]-3-hydroquinazolin-4-one; 3-(2-Fluoro-phenyl)-2-[1-(2-fluoro-9H-purin-6-ylamino)-ethyl]-5-methyl-3-hydroquinazolin-4-one; 2-[1-(6-aminopurin-9-yl)ethyl]-3-(3,5-difluorophenyl)-5-methyl-3-hydroquinazolin-4-one; 2-[1-(7-Amino-[1,2,3]triazolo[4,5-d]pyrimidin-3-yl)-ethyl]-3-(3,5-difluoro-phenyl)-5-methyl-3H-quinazolin-4-one; 5-chloro-3-(3,5-difluoro-phenyl)-2-[1-(9H-purin-6-ylamino)-propyl]-3H-quinazolin-4-one; 3-phenyl-2-[1-(9H-purin-6-ylamino)-propyl]-3H-quinazolin-4-one; 5-fluoro-3-phenyl-2-[1-(9H-purin-6-ylamino)-propyl]-3H-quinazolin-4-one; 3-(2,6-difluoro-phenyl)-5-methyl-2-[1-(9H-purin-6-ylamino)-propyl]-3H-quinazolin-4-one; 6-fluoro-3-phenyl-2-[1-(9H-purin-6-ylamino)-ethyl]-3H-quinazolin-4-one; 3-(3,5-difluoro-phenyl)-5-methyl-2-[1-(9H-purin-6-ylamino)-ethyl]-3H-quinazolin-4-one; 5-fluoro-3-phenyl-2-[1-(9H-purin-6-ylamino)-ethyl]-3H-quinazolin-4-one; 3-(2,3-difluoro-phenyl)-5-methyl-2-[1-(9H-purin-6-ylamino)-ethyl]-3H-quinazolin-4-one; 5-methyl-3-phenyl-2-[1-(9H-purin-6-ylamino)-ethyl]-3H-quinazolin-4-one; 3-(3-chloro-phenyl)-5-methyl-2-[1-(9H-purin-6-ylamino)-ethyl]-3H-quinazolin-4-one; 5-methyl-3-phenyl-2-[(9H-purin-6-ylamino)-methyl]-3H-quinazolin-4-one; 2-[(2-amino-9H-purin-6-ylamino)-methyl]-3-(3,5-difluoro-phenyl)-5-methyl-3H-quinazolin-4-one; 3-{2-[(2-diethylamino-ethyl)-methyl-amino]-phenyl}-5-methyl-2-[(9H-purin-6-ylamino)-methyl]-3H-quinazolin-4-one; 5-chloro-3-(2-fluoro-phenyl)-2-[(9H-purin-6-ylamino)-methyl]-3H-quinazolin-4-one; 5-chloro-2-[(9H-purin-6-ylamino)-methyl]-3-o-tolyl-3H-quinazolin-4-one; 5-chloro-3-(2-chloro-phenyl)-2-[(9H-purin-6-ylamino)-methyl]-3H-quinazolin-4-one; 6-fluoro-3-(3-fluoro-phenyl)-2-[1-(9H-purin-6-ylamino)-ethyl]-3H-quinazolin-4-one; 2-[1-(2-amino-9H-purin-6-ylamino)-ethyl]-5-chloro-3-(3-fluoro-phenyl)-3H-quinazolin-4-one; 5-methyl-3-phenyl-2-[1-(9H-purin-6-ylamino)-propyl]-3H-quinazolin-4-one; 2-[1-(2-fluoro-9H-purin-6-ylamino)-ethyl]-5-methyl-3-phenyl-3H-quinazolin-4-one; 3-(2,6-difluoro-phenyl)-5-methyl-2-[1-(9H-purin-6-ylamino)-ethyl]-3H-quinazolin-4-one; 2-[1-(2-amino-9H-purin-6-ylamino)-ethyl]-3-(2,6-difluoro-phenyl)-5-methyl-3H-quinazolin-4-one; 3-(2,6-difluoro-phenyl)-2-[1-(2-fluoro-9H-purin-6-ylamino)-ethyl]-5-methyl-3H-quinazolin-4-one; 3-(2,6-difluoro-phenyl)-5-methyl-2-[1-(7H-pyrrolo[2,3-d]pyrimidin-4-ylamino)-ethyl]-3H-quinazolin-4-one; 2-[1-(2-amino-9H-purin-6-ylamino)-propyl]-5-methyl-3-phenyl-3H-quinazolin-4-one; 5-methyl-3-phenyl-2-[1-(7H-pyrrolo[2,3-d]pyrimidin-4-ylamino)-propyl]-3H-quinazolin-4-one; 2-[1-(2-fluoro-9h-purin-6-ylamino)-propyl]-5-methyl-3-phenyl-3h-quinazolin-4-one; 5-methyl-3-phenyl-2-[1-(9H-purin-6-ylamino)-ethyl]-3H-quinazolin-4-one; 2-[1-(2-amino-9H-purin-6-ylamino)-ethyl]-5-methyl-3-phenyl-3H-quinazolin-4-one; 2-[2-benzyloxy-1-(9H-purin-6-ylamino)-ethyl]-5-methyl-3-phenyl-3H-quinazolin-4-one; 2-[1-(2-amino-9H-purin-6-ylamino)-2-benzyloxy-ethyl]-5-methyl-3-phenyl-3H-quinazolin-4-one; 2-[2-benzyloxy-1-(7H-pyrrolo[2,3-d]pyrimidin-4-ylamino)-ethyl]-5-methyl-3-phenyl-3H-quinazolin-4-one; 2-[2-benzyloxy-1-(2-fluoro-9H-purin-6-ylamino)-ethyl]-5-methyl-3-phenyl-3H-quinazolin-4-one; 3-(4-fluoro-phenyl)-5-methyl-2-[1-(9H-purin-6-ylamino)-ethyl]-3H-quinazolin-4-one; 2-[1-(2-amino-9H-purin-6-ylamino)-ethyl]-3-(4-fluoro-phenyl)-5-methyl-3H-quinazolin-4-one; 3-(4-fluoro-phenyl)-2-[1-(2-fluoro-9H-purin-6-ylamino)-ethyl]-5-methyl-3H-quinazolin-4-one; 3-(4-fluoro-phenyl)-5-methyl-2-[1-(7H-pyrrolo[2,3-d]pyrimidin-4-ylamino)-ethyl]-3H-quinazolin-4-one; 5-methyl-3-phenyl-2-[1-(7H-pyrrolo[2,3-d]pyrimidin-4-ylamino)-ethyl]-3H-quinazolin-4-one; 3-(3-fluoro-phenyl)-5-methyl-2-[1-(9H-purin-6-ylamino)-ethyl]-3H-quinazolin-4-one; 2-[1-(2-amino-9H-purin-6-ylamino)-ethyl]-3-(3-fluoro-phenyl)-5-methyl-3H-quinazolin-4-one; 3-(3-fluoro-phenyl)-5-methyl-2-[1-(7H-pyrrolo[2,3-d]pyrimidin-4-ylamino)-ethyl]-3H-quinazolin-4-one; 5-methyl-3-phenyl-2-[1-(9H-purin-6-yl)-pyrrolidin-2-yl]-3H-quinazolin-4-one; 2-[2-hydroxy-1-(9H-purin-6-ylamino)-ethyl]-5-methyl-3-phenyl-3H-quinazolin-4-one; 5-methyl-3-phenyl-2-[phenyl-(9H-purin-6-ylamino)-methyl]-3H-quinazolin-4-one; 2-[(2-amino-9H-purin-6-ylamino)-phenyl-methyl]-5-methyl-3-phenyl-3H-quinazolin-4-one; 2-[(2-fluoro-9H-purin-6-ylamino)-phenyl-methyl]-5-methyl-3-phenyl-3H-quinazolin-4-one; 5-methyl-3-phenyl-2-[phenyl-(7H-pyrrolo[2,3-d]pyrimidin-4-ylamino)-methyl]-3H-quinazolin-4-one; 5-fluoro-3-phenyl-2-[1-(9H-purin-6-ylamino)-ethyl]-3H-quinazolin-4-one; 2-[1-(2-amino-9H-purin-6-ylamino)-ethyl]-5-fluoro-3-phenyl-3H-quinazolin-4-one; 2-[1-(2-amino-9H-purin-6-ylamino)-ethyl]-5-chloro-3-phenyl-3H-quinazolin-4-one; [5-(5-methyl-4-oxo-3-phenyl-3,4-dihydro-quinazolin-2-yl)-5-(9H-purin-6-ylamino)-pentyl]-carbamic acid benzyl ester; [5-(2-amino-9H-purin-6-ylamino)-5-(5-methyl-4-oxo-3-phenyl-3,4-dihydro-quinazolin-2-yl)-pentyl]-carbamic acid benzyl ester; [4-(5-methyl-4-oxo-3-phenyl-3,4-dihydro-quinazolin-2-yl)-4-(9H-purin-6-ylamino)-butyl]-carbamic acid benzyl ester; [4-(2-amino-9H-purin-6-ylamino)-4-(5-methyl-4-oxo-3-phenyl-3,4-dihydro-quinazolin-2-yl)-butyl]-carbamic acid benzyl ester; 3-phenyl-2-[1-(9H-purin-6-ylamino)-ethyl]-3H-quinazolin-4-one; 2-[5-amino-1-(9H-purin-6-ylamino)-pentyl]-5-methyl-3-phenyl-3H-quinazolin-4-one); 2-[5-amino-1-(2-amino-9H-purin-6-ylamino)-pentyl]-5-methyl-3-phenyl-3H-quinazolin-4-one; 2-[1-(2-amino-9H-purin-6-ylamino)-ethyl]-3-(2,6-Dimethyl-phenyl)-5-methyl-3H-quinazolin-4-one; 3-(2,6-dimethyl-phenyl)-5-methyl-2-[1-(9H-purin-6-ylamino)-ethyl]-3H-quinazolin-4-one; 5-morpholin-4-ylmethyl-3-phenyl-2-[1-(9H-purin-6-ylamino)-ethyl]-3H-quinazolin-4-one; 2-[1-(2-amino-9H-purin-6-ylamino)-ethyl]-5-morpholin-4ylmethyl-3-phenyl-3H-quinazolin-4-one; 2-[4-amino-1-(2-amino-9H-purin-6-ylamino)-butyl]-5-methyl-3-phenyl-3H-quinazolin-4-one; 6-fluoro-3-phenyl-2-[1-(9H-purin-6-ylamino)-ethyl]-3H-quinazolin-4-one; 2-[1-(2-amino-9H-purin-6-ylamino)-ethyl]-6-fluoro-3-phenyl-3H-quinazolin-4-one; 2-[2-tert-butoxy-1-(9H-purin-6-ylamino)-ethyl]-5-methyl-3-phenyl-3H-quinazolin-4-one; 3-(3-methyl-phenyl)-5-methyl-2-[1-(9H-purin-6-ylamino)-ethyl]-3H-quinazolin-4-one; 2-[1-(2-amino-9H-purin-6-ylamino)-ethyl]-3-(3-methyl-phenyl)-5-methyl-3H-quinazolin-4-one; 3-(3-chloro-phenyl)-5-methyl-2-[1-(9H-purin-6-ylamino)-ethyl]-3H-quinazolin-4-one; 2-[1-(2-amino-9H-purin-6-ylamino)-ethyl]-3-(3-chloro-phenyl)-5-methyl-3H-quinazolin-4-one; 2-[1-(2-amino-9H-purin-6-ylamino)-2-hydroxy-ethyl]-5-methyl-3-phenyl-3H-quinazolin-4-one; 2-[1-(2-amino-9H-purin-6-ylamino)-ethyl]-3-(3-fluoro-phenyl)-3H-quinazolin-4-one; 2-[1-(2-amino-9H-purin-6-ylamino)-ethyl]-3-(2,6-difluoro-phenyl)-3H-quinazolin-4-one; 2-[1-(2-amino-9H-purin-6-ylamino)-propyl]-5-fluoro-3-phenyl-3H-quinazolin-4-one; 5-chloro-3-(3-fluoro-phenyl)-2-[1-(9H-purin-6-ylamino)-ethyl]-3H-quinazolin-4-one; 2-[1-(2-amino-9H-purin-6-ylamino)-ethyl]-5-chloro-3-(3-fluoro-phenyl)-3H-quinazolin-4-one; 3-phenyl-2-[1-(9H-purin-6-ylamino)-ethyl]-5-trifluoromethyl-3H-quinazolin-4-one; 3-(2,6-difluoro-phenyl)-5-methyl-2-[1-(9H-purin-6-ylamino)-propyl]-3H-quinazolin-4-one; 3-(2,6-difluoro-phenyl)-5-methyl-2-[1-(9H-purin-6-ylamino)-ethyl]-3H-quinazolin-4-one; 2-[1-(2-amino-9H-purin-6-ylamino)-propyl]-3-(2,6-difluoro-phenyl)-5-methyl-3H-quinazolin-4-one; 2-[1-(2-amino-9H-purin-6-ylamino)-ethyl]-3-(2,6-difluoro-phenyl)-5-methyl-3H-quinazolin-4-one; 3-(3,5-dichloro-phenyl)-5-methyl-2-[1-(9H-purin-6-ylamino)-ethyl]-3H-quinazolin-4-one; 3-(2,6-dichloro-phenyl)-5-methyl-2-[1-(9H-purin-6-ylamino)-ethyl]-3H-quinazolin-4-one; 2-[1-(2-amino-9H-purin-6-ylamino)-ethyl]-3-(2,6-dichloro-phenyl)-5-methyl-3H-quinazolin-4-one; 5-chloro-3-phenyl-2-[1-(9H-purin-6-ylamino)-propyl]-3H-quinazolin-4-one; 2-[1-(2-amino-9H-purin-6-ylamino)-propyl]-5-chloro-3-phenyl-3H-quinazolin-4-one; 5-methyl-3-phenyl-2-[1-(9H-purin-6-ylamino)-butyl]-3H-quinazolin-4-one; 2-[1-(2-amino-9H-purin-6-ylamino)-butyl]-5-methyl-3-phenyl-3H-quinazolin-4-one; 2-[1-(2-amino-9H-purin-6-ylamino)-ethyl]-3-(3,5-dichloro-phenyl)-5-methyl-3H-quinazolin-4-one; 5-methyl-3-(3-morpholin-4-ylmethyl-phenyl)-2-[1-(9H-purin-6-ylamino)-ethyl]-3H-quinazolin-4-one; 2-[1-(2-amino-9H-purin-6-ylamino)-ethyl]-5-methyl-3-(3-morpholin-4-ylmethyl-phenyl)-3H-quinazolin-4-one; 2-[1-(5-bromo-7H-pyrrolo[2,3-d]pyrimidin-4-ylamino)-ethyl]-5-methyl-3-phenyl-3H-quinazolin-4-one; 5-methyl-2-[1-(5-methyl-7H-pyrrolo[2,3-d]pyrimidin-4-ylamino)-ethyl]-3-phenyl-3H-quinazolin-4-one; 2-[1-(5-fluoro-7H-pyrrolo[2,3-d]pyrimidin-4-ylamino)-ethyl]-5-methyl-3-phenyl-3H-quinazolin-4-one; 2-[2-hydroxy-1-(9H-purin-6-ylamino)-ethyl]-3-phenyl-3H-quinazolin-4-one; 3-(3,5-difluoro-phenyl)-5-methyl-2-[1-(9H-purin-6-ylamino)-propyl]-3H-quinazolin-4-one; 2-[1-(2-amino-9H-purin-6-ylamino)-propyl]-3-(3,5-difluoro-phenyl)-5-methyl-3H-quinazolin-4-one; 3-(3,5-difluoro-phenyl)-2-[1-(9H-purin-6-ylamino)-ethyl]-3H-quinazolin-4-one; 2-[1-(5-bromo-7H-pyrrolo[2,3-d]pyrimidin-4-ylamino)-ethyl]-3-(3-fluoro-phenyl)-5-methyl-3H-quinazolin-4-one; 3-(3-fluoro-phenyl)-5-methyl-2-[1-(5-methyl-7H-pyrrolo[2,3-d]pyrimidin-4-ylamino)-ethyl]-3H-quinazolin-4-one; 3-phenyl-2-[1-(9H-purin-6-ylamino)-propyl]-3H-quinazolin-4-one; 2-[1-(2-amino-9H-purin-6-ylamino)-ethyl]-3-(3,5-difluoro-phenyl)-3H-quinazolin-4-one; 2-[1-(2-amino-9H-purin-6-ylamino)-propyl]-3-phenyl-3H-quinazolin-4-one; 6,7-difluoro-3-phenyl-2-[1-(9H-purin-6-ylamino)-ethyl]-3H-quinazolin-4-one; 6-fluoro-3-(3-fluoro-phenyl)-2-[1-(9H-purin-6-ylamino)-ethyl]-3H-quinazolin-4-one; 2-[4-diethylamino-1-(9H-purin-6-ylamino)-butyl]-5-methyl-3-phenyl-3H-quinazolin-4-one; 5-fluoro-3-phenyl-2-[1-(9H-purin-6-ylamino)-propyl]-3H-quinazolin-4-one; 3-phenyl-2-[1-(9H-purin-6-ylamino)-ethyl]-3H-quinazolin-4-one; 6-fluoro-3-phenyl-2-[1-(9H-purin-6-ylamino)-ethyl]-3H-quinazolin-4-one; 3-(3,5-difluoro-phenyl)-5-methyl-2-[1-(9H-purin-6-ylamino)-ethyl]-3H-quinazolin-4-one; 5-fluoro-2-[1-(2-fluoro-9H-purin-6-ylamino)-ethyl]-3-phenyl-3H-quinazolin-4-one; 3-(3-fluoro-phenyl)-2-[1-(9H-purin-6-ylamino)-ethyl]-3H-quinazolin-4-one; 5-chloro-3-(3,5-difluoro-phenyl)-2-[1-(9H-purin-6-ylamino)-propyl]-3H-quinazolin-4-one; 3-(2,6-difluoro-phenyl)-5-methyl-2-[1-(9H-purin-6-ylamino)-ethyl]-3H-quinazolin-4-one; 3-(2,6-difluoro-phenyl)-2-[1-(9H-purin-6-ylamino)-ethyl]-3H-quinazolin-4-one; 5-Methyl-3-phenyl-2-[3,3,3-trifluoro-1-(9H-purin-6-ylamino)-propyl]-3H-quinazolin-4-one; 3-(3-hydroxy-phenyl)-5-methyl-2-[1-(9H-purin-6-ylamino)-ethyl]-3H-quinazolin-4-one; 3-(3-methoxy-phenyl)-5-methyl-2-{1-[9H-purin-6-ylamino]-ethyl}-3H-quinazolin-4-one; 3-[3-(2-dimethylamino-ethoxy)-phenyl]-5-methyl-2-{1-[9H-purin-6-ylamino]-ethyl}-3H-quinazolin-4-one; 3-(3-cyclopropylmethoxy-phenyl)-5-methyl-2-{1-[9H-purin-6-ylamino]-ethyl}-3H-quinazolin-4-one; 5-methyl-3-(3-prop-2-ynyloxy-phenyl)-2-{1-[9H-purin-6-ylamino]-ethyl}-3H-quinazolin-4-one; 2-{1-[2-amino-9H-purin-6-ylamino]ethyl}-3-(3-hydroxyphenyl)-5-methyl-3H-quinazolin-4-one; 2-{1-[2-amino-9H-purin-6-ylamino]ethyl}-3-(3-methoxyphenyl)-5-methyl-3H-quinazolin-4-one; 2-{1-[2-amino-9H-purin-6-ylamino]ethyl}-3-(3-cyclopropylmethoxy-phenyl)-5-methyl-3H-quinazolin-4-one; 2-{1-[2-amino-9H-purin-6-ylamino]ethyl}-5-methyl-3-(3-prop-2-ynyloxy-phenyl)-3H-quinazolin-4-one; 3-(3-ethynyl-phenyl)-5-methyl-2-[1-(9H-purin-6-ylamino)-ethyl]-3H-quinazolin-4-one; 3-{5-methyl-4-oxo-2-[1-(9H-purin-6-ylamino)-ethyl]-4H-quinazolin-3-yl}-benzonitrile; 3-{5-methyl-4-oxo-2-{1-[9H-purin-6-ylamino)-ethyl]-4H-quinazolin-3-yl}benzamide; 3-(3-acetyl-phenyl)-5-methyl-2-{1-[9H-purin-6-ylamino]-ethyl}-3H-quinazolin-4-one; 2-(3-(5-methyl-4-oxo-2-{1-[9H-purin-6-ylamino]-ethyl}-4H-quinazolin-3-yl-phenoxy acetamide; 5-methyl-2-{1-[9H-purin-6-ylamino]-ethyl}-3-[3-(tetrahydropuran-4-yloxy)-phenyl]-3H-quinazolin-4-one; 3-[3-(2-methoxy-ethoxy)-phenyl]-5-methyl-2-[1-(9H-purin-6-ylamino)-ethyl]-3H-quinazolin-4-one; 6-fluoro-2-[1-(9H-purin-6-ylamino)ethyl]-3-[3-(tetrahydro-pyran-4-yloxy)-phenyl]-3H-quinazolin-4-one; 3-[3-(3-dimethylamino-propoxy)-phenyl]-5-methyl-2-[1-(9H-purin-6-ylamino)-ethyl]-3H-quinazolin-4-one; 2-[1-(2-amino-9H-purin-6-ylamino)-ethyl]-3-(3-ethynyl-phenyl)-5-methyl-3H-quinazolin-4-one; 3-{2-[1-(2-amino-9H-purin-6-ylamino)-ethyl]-5-methyl-4-oxo-4H-quinazolin-3-yl}-benzonitrile; 3-{2-[1-(2-amino-9H-purin-6-ylamino)-ethyl]-5-methyl-4-oxo-4H-quinazolin-3-yl}-benzamide; 3-{2-[1-(2-amino-9H-purin-6-ylamino)-ethyl]-5-methyl-4-oxo-4H-quinazolin-3-yl}-benzamide; 5-methyl-3-(3-morpholin-4-yl-phenyl)-2-[1-(9H-purin-6-ylamino)-ethyl]-3H-quinazolin-4-one; 2-[1-(2-amino-9H-purin-6-ylamino)-ethyl]-5-methyl-3-(3-morpholin-4-yl-phenyl)-3H-quinazolin-4-one; 2-[1-(2-amino-9H-purin-6-ylamino)-ethyl]-3-[3-(2-methoxy-ethoxy)-phenyl]-5-methyl-3H-quinazolin-4-one; 2-[1-(2-amino-9H-purin-6-ylamino)-ethyl]-3-[3-(2-dimethylamino-ethoxy)-phenyl]-5-methyl-3H-quinazolin-4-one; 2-[1-(2-amino-9H-purin-6-ylamino)-but-3-ynyl]-5-methyl-3-phenyl-3H-quinazolin-4-one; 2-[1-(2-amino-9H-purin-6-ylamino)-but-3-ynyl]-5-methyl-3-phenyl-3H-quinazolin-4-one; 5-chloro-3-(3,5-difluoro-phenyl)-2-[1-(9H-purin-6-ylamino)-ethyl]-3H-quinazolin-4-one; 2-[1-(2-amino-9H-purin-6-ylamino)-propyl]-5-chloro-3-(3,5-difluoro-phenyl)-3H-quinazolin-4-one; 2-[1-(2-amino-9H-purin-6-ylamino)-ethyl]-5-chloro-3-(3,5-difluoro-phenyl)-3H-quinazolin-4-one; 3-(3,5-difluoro-phenyl)-6-fluoro-2-[1-(9H-purin-6-ylamino)-ethyl]-3H-quinazolin-4-one; 5-chloro-3-(2,6-difluoro-phenyl)-2-[1-(9H-purin-6-ylamino)-propyl]-3H-quinazolin-4-one; 2-[1-(2-amino-9H-purin-6-ylamino)-propyl]-5-chloro-3-(2,6-difluoro-phenyl)-3H-quinazolin-4-one; 5-methyl-3-phenyl-2-[1-(9H-purin-6-yloxy)-ethyl]-3H-quinazolin-4-one; and,
pharmaceutically acceptable salts and solvates thereof.

31. An article of manufacture comprising a phosphoinositide 3-kinase delta (PI3Kδ) selective inhibitor and a label indicating a method according to claim 2.

32. An article of manufacture comprising a phosphoinositide 3-kinase delta (PI3Kδ) selective inhibitor and a label indicating a method according to claim 18.
